(19) United States
(12) Patent Application Publication
(10) Pub. No.: US 2026/0337603 A1
(43) Pub. Date: Oct. 29, 2026

(54) METHODS AND SYSTEMS FOR KINSHIP EVALUATION FOR MISSING PERSONS AND DISASTER/CONFLICT VICTIMS

(71) Applicant: Verogen, Inc., San Diego, CA (US)

(72) Inventors: June SNEDECOR, San Diego, CA (US); Kathryn M. STEPHENS, San Diego, CA (US); Sarah M. RADECKE, San Diego, CA (US); Gothami PADMABANDU, San Diego, CA (US); Joana Alexandra Pereira ANTUNES, San Diego, CA (US)

(73) Assignee: Verogen, Inc., San Diego, CA (US)

(21) Appl. No.: 19/103,731

(22) PCT Filed: Aug. 15, 2023
(86) PCT No.: PCT/US2023/072246

§ 371 Date: Feb. 13, 2025

Related U.S. Application Data
(60) Provisional application No. 63/398,512, filed on Aug. 16, 2022.
(60) Provisional application No. 63/445,541, filed on Feb. 14, 2023.

(51) Int. Cl. C12N 15/10; C12Q 1/6827; C12Q 1/686; C12Q 1/6876; G16B 20/20; G16B 40/30

(52) U.S. Cl. CPC C12N 15/1093; C12Q 1/6827; C12Q 1/686; C12Q 1/6876; G16B 20/20; G16B 40/30; C12Q 2600/156; C12Q 2600/16

(57) ABSTRACT

The present disclosure in some aspects relates to performing DNA based kinship analysis involving analysis of between 2,000 and 50,000 SNPs, including sample preparation and sequencing technologies and methods that can be used to calculate the degree of relationship of a DNA profile from a person of interest, such as a missing person or a victim of a conflict or disaster, to one or more reference DNA profiles in a reference set of DNA profiles that includes at least one DNA profile from a relative of the person of interest.

### CROSS-REFERENCE TO RELATED APPLICATIONS

This application claims priority from U.S. provisional application No. 63/398,512, filed Aug. 16, 2022, entitled “METHODS AND SYSTEMS FOR KINSHIP EVALUATION FOR MISSING PERSONS AND DISASTER/CONFLICT VICTIMS,” and U.S. provisional application No. 63/445,541, filed Feb. 14, 2023, entitled “METHODS AND SYSTEMS FOR KINSHIP EVALUATION FOR MISSING PERSONS AND DISASTER/CONFLICT VICTIMS,” the contents of which are incorporated by reference in their entirety.

### FIELD

The present disclosure relates in some aspects to methods and systems for DNA-based kinship evaluations for persons of interest, such as missing persons and victims of conflicts and disasters.

### BACKGROUND

Current methods of generating DNA profiles for comparisons in genetic databases include genotyping using dense SNP microarrays and whole genome sequencing (WGS) followed by association of evidentiary samples with distant relatives in databases, which require high quantity and high quality DNA samples, and are not designed for familial searching or forensic purposes. Forensic casework samples are generally low quantity and low quality samples, and typically require querying a publicly accessible database genetic database to identify familial relationships. However, in situations of missing persons or victims of a disaster or conflict, family members may be hesitant to provide a genetic sample that would aid in identifying such a person if it would result in their genetic sample being uploaded onto a publicly accessible database. Therefore, there is need for a new and improved method for the generation of DNA based profile analysis that allows for identifying familial relationships of persons of interest, such as missing persons or victims of a disaster or conflict, without requiring the use of a publicly accessible genetic database.

### SUMMARY

Provided herein is a method for performing DNA-based kinship analysis, comprising: providing a nucleic acid sample from a person of interest, amplifying the nucleic acid sample with a plurality of primers that specifically hybridize to a plurality of target sequences collectively comprising a plurality of at least between at or about 2,000 to 50,000 single nucleotide polymorphisms (SNPs), thereby generating amplification products, wherein the amplification is carried out in one or more multiplex PCR reactions, generating a nucleic acid library from the amplification products, sequencing the nucleic acid library generated from the amplification products, analyzing the sequences of the amplification products, determining the genotypes of the plurality of SNPs, thereby generating a DNA profile, and calculating the degree of relationship of the DNA profile to one or more reference DNA profiles, wherein the one or more reference DNA profiles are comprised within a reference set of DNA profiles comprising one or more reference DNA profiles from a relative of the person of interest.

Also provided herein is a method for performing DNA-based kinship analysis, comprising: providing a nucleic acid sample from a person of interest, amplifying the nucleic acid sample with a plurality of primers that specifically hybridize to a plurality of target sequences collectively comprising a plurality of at least between at or about 2,000 to 50,000 single nucleotide polymorphisms (SNPs), thereby generating amplification products, wherein the amplification is carried out in one or more multiplex PCR reactions, generating a nucleic acid library from the amplification products, sequencing the nucleic acid library generated from the amplification products, determining the genotypes of the plurality of SNPs, thereby generating a DNA profile, and calculating the degree of relationship of the DNA profile to one or more reference DNA profiles, wherein the one or more reference DNA profiles are comprised within a reference set of DNA profiles comprising one or more reference DNA profiles from a relative of the person of interest.

In some of any of such embodiments, the sequencing is conducted using massively parallel sequencing (MPS). In some of any of such embodiments, the sequencing does not comprise whole genome sequencing (WGS).

In some of any of such embodiments, the method further comprises generating a family tree comprising the DNA profile in relation to one or more DNA profiles.

Also provided herein is a method of constructing a nucleic acid library for a person of interest, comprising: providing a nucleic acid sample from a person of interest, amplifying the nucleic acid sample with a plurality of primers that specifically hybridize to a plurality of target sequences collectively comprising a plurality of at least between at or about 2,000 to 50,000 single nucleotide polymorphisms (SNPs), thereby generating a nucleic acid library comprising amplification products, wherein the amplification is carried out in one or more multiplex PCR reactions. In some embodiments, the method further comprises a step of sequencing the amplification products to produce a DNA profile for the person of interest.

Also provided herein is a method of constructing a nucleic acid library for a reference DNA sample, comprising: providing a nucleic acid sample from a relative of a person of interest, amplifying the nucleic acid sample with a plurality of primers that specifically hybridize to a plurality of target sequences collectively comprising a plurality of at least between at or about 2,000 to 50,000 single nucleotide polymorphisms (SNPs), thereby generating a nucleic acid library comprising amplification products, wherein the amplification is carried out in one or more multiplex PCR reactions.

In some embodiments, the relative is a first-, second-, third-, fourth-, or fifth-degree relative of the person of interest. In some of any of such embodiments, the relative is a first-, second-, or third-degree relative of the person of interest.

In some of any of such embodiments, the nucleic acid sample comprises genomic DNA. In some of any of such embodiments, the nucleic acid sample comprises one or more enzyme inhibitors. In some of any of such embodiments, the one or more enzyme inhibitors comprise one or more inhibitors selected from the group consisting of hematin, heme, humic acid, indigo, tannic acid, collagen, calcium, and hydroxyapatite. In some of any of such embodiments, the nucleic acid sample comprises low-quality nucleic acid molecules and/or low quantity nucleic acid molecules. In some of any of such embodiments, the low quality nucleic acid molecules are degraded genomic DNA and/or fragmented genomic DNA. In some of any of such embodiments, the low quality nucleic acid molecules have a degradation index (DI) of at or at least 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200. In some of any of such embodiments, the low quality nucleic acid molecules have a DI of at least 1 and up to or less than 158.3. In some of any of such embodiments, the nucleic acid sample comprises high quality nucleic acid molecules. In some of any of such embodiments, the high quality nucleic acid molecules have a DI of less than 1.

In some of any of such embodiments, the person of interest is a missing person. In some of any of such embodiments, the person of interest a victim of a disaster or conflict.

In some of any of such embodiments, the nucleic acid sample is derived from saliva, blood, semen, hair, teeth, bone, or skin In some of any of such embodiments, the nucleic acid sample is derived from saliva, blood, or semen. In some of any of such embodiments, the nucleic acid sample is derived from bone or hair. In some of any of such embodiments, the nucleic acid sample is derived from a buccal swab, paper, fabric, or other substrate or object that is impregnated with saliva, blood, semen, or other bodily fluid, or contains hair or skin cells.

In some of any of such embodiments, the nucleic acid sample comprises between or between about 3 pg and 100 ng of genomic DNA. In some of any of such embodiments, the nucleic acid sample comprises between or between about 100 pg and 5 ng of genomic DNA, between or between about 50 pg and 5 ng of genomic DNA, or between or between about 3 pg and 5 ng of genomic DNA. In some of any of such embodiments, the nucleic acid sample comprises at or about 1 ng of genomic DNA.

In some of any of such embodiments, the plurality of SNPs comprises kinship SNPs (kiSNPs). In some of any of such embodiments, the plurality of SNPs comprises Y-chromosome SNPs (Y-SNPs). In some of any of such embodiments, the plurality of SNPs comprises kiSNPs and Y-SNPs. In some of any of such embodiments, the plurality of SNPs comprises kiSNPs, biogeographical ancestry SNPs (aiSNPs), identity SNPs (iiSNPs), phenotype SNPs (piSNPs), X-chromosome SNPs (X-SNPs), and Y-chromosome SNPs (Y-SNPs). In some of any of such embodiments, the plurality of SNPs comprises SNPs selected from one or more of the groups consisting of kiSNPs, aiSNPs, iiSNPs, piSNPs, X-SNPs, and Y-SNPs. In some of any of such embodiments, at least or at least about 80%, 81%, 82%, 83%, 84%, 85%, 86%, 87%, 88%, 89%, 90%, 91%, 92%, 93%, 94%, 95%, 96%, 97%, 98%, or 99% of the plurality of SNPs are kinship SNPs.

In some of any of such embodiments, the reference set of DNA profiles comprises up to 5, 10, 15, 20, 25, 30, 35, 40, 45, 50, 55, 60, 65, 70, 75, 80, 85, 90, 95, 100, 125, 150, 175, 200, 300, 400, 500, 600, 700, 800, 900, or 1000 reference DNA profiles. In some of any of such embodiments, at least 5%, 10%, 15%, 20%, 25%, 30%, 35%, 40%, 45%, 50%, 55%, 60%, 65%, 70%, 75%, 80%, 85%, 90%, or 95% of the reference DNA profiles in the reference set of DNA profiles is from a relative of the person of interest. In some of any of such embodiments, at least 5%, 10%, 15%, 20%, 25%, 30%, 35%, 40%, 45%, 50%, 55%, 60%, 65%, 70%, 75%, 80%, 85%, 90%, or 95% of the reference DNA profiles in the reference set of DNA profiles is from a relative of the person of interest, and wherein each of the at least 5%, 10%, 15%, 20%, 25%, 30%, 35%, 40%, 45%, 50%, 55%, 60%, 65%, 70%, 75%, 80%, 85%, 90%, or 95% of the reference DNA profiles in the reference set of DNA profiles is a first degree, second degree, third degree, fourth degree, or fifth degree relative. In some of any of such embodiments, at least 50% of the reference DNA profiles in the reference set of DNA profiles is from a relative of the person of interest.

In some of any of such embodiments, each relative of the person of interest in the reference set of DNA profiles is individually a first degree, second degree, third degree, fourth degree, or fifth degree relative of the person of interest. In some of any of such embodiments, each relative of the person of interest in the reference set of DNA profiles is individually a first degree, second degree, or third degree relative of the person of interest. In some of any of such embodiments, the identity of each relative of the person of interest in the reference set of DNA profiles is known. In some of any of such embodiments, the identity of each of the one or more reference DNA profiles in the reference set of DNA profiles is known. In some of any of such embodiments, the reference set of DNA profiles is in a database. In some embodiments, the database is not publicly accessible.

In some of any of such embodiments, the sequencing comprises a sequencing plexity of up to 40-plex. In some of any of such embodiments, the sequencing comprises a sequencing plexity of up to 32-plex. In some of any of such embodiments, the sequencing comprises a sequencing plexity of 12-plex to 32-plex. In some of any of such embodiments, the sequencing comprises a sequencing plexity of 24-plex to 32-plex. In some of any of such embodiments, the sequencing comprises a sequencing plexity of at or about 10-plex, 11-plex, 12-plex, 13-plex, 14-plex, 15-plex, 16-plex, 17-plex 18-plex, 19-plex, 20-plex, 21-plex, 22-plex, 23-plex, 24-plex, 25-plex, 26-plex, 27-plex, 28-plex, 29-plex, 30-plex, 31-plex, 32-plex, 33-plex, 34-plex, or 35-plex. In some of any of such embodiments, the sequencing comprises a sequencing plexity of at or about 8-to 16-plex for postmortem samples, and/or the sequencing comprises a sequencing plexity of at or about 24-to 40-plex for antemortem samples. In some of any of such embodiments, the sequencing comprises a sequencing plexity of at or about 12-plex for postmortem samples, and/or the sequencing comprises a sequencing plexity of at or about 32-plex for antemortem samples. In some of any of such embodiments, the sequencing comprises a sequencing plexity of at or about 30-plex, 31-plex, or 32-plex.

In some of any of such embodiments, the method further comprises identifying the person of interest.

Also provided herein is a method for calculating degree of relatedness, comprising: obtaining a DNA profile comprising genotypes of at least between at or about 2,000 to 50,000 SNPs, wherein the DNA profile is from a person of interest; and calculating the degree of relationship of the DNA profile to one or more reference DNA profiles, wherein the one or more reference DNA profiles are comprised within a reference set of DNA profiles comprising one or more reference DNA profiles from a relative of the person of interest.

Also provided herein is a method for calculating degree of relatedness, comprising: generating a DNA profile comprising genotypes of at least between at or about 2,000 to 50,000 SNPs, wherein the DNA profile is from a person of interest; and calculating the degree of relationship of the DNA profile to one or more reference DNA profiles, wherein the one or more reference DNA profiles are comprised within a reference set of DNA profiles comprising one or more reference DNA profiles from a relative of the person of interest.

In some of any of such embodiments, the degree of relationship is calculated using a kinship model. In some of any of such embodiments, the degree of relationship is calculated using a kinship model that is trained using a PCA method. In some embodiments, the PCA method for training the kinship model is PCA or involves PCA. In some of any of such embodiments, the PCA method is PC-AiR. In some embodiments, the PC-AiR comprises the steps of: (1) estimating kinship coefficients between all pairs of samples, optionally training DNA profiles, of a training database, wherein pairings with a kinship coefficient >0.025 are identified as related and pairings with a kinship coefficient <−0.025 are identified as ancestry-diverged; (2) initializing an unrelated sample set that includes all samples; and (3) iteratively: (i) identifying the set in the unrelated sample set that have the most related samples in the unrelated sample set, thereby designated as X, (ii) identifying the set of samples in X that have the least ancestry-diverged pairings compared to samples in the unrelated sample set, thereby designated as Y; and (iii) if Y has zero samples, then terminating the process, or, if Y has at least one sample, then randomly selecting one sample from Y to remove from U, and repeat beginning at step (3) (i).

In some of any of such embodiments, the PCA method is a modified PC-Air. In some of any of such embodiments, the modified PC-AiR comprises steps of: (1) estimating kinship coefficients between all pairs of samples, optionally training DNA profiles, of a training database, wherein pairings with a kinship coefficient >0.01 are identified as related and pairings with a kinship coefficient <−0.025 are identified as ancestry-diverged; (2) removing all DNA profiles that have ≥5% missing data; and (3) ranking all DNA profiles by identifying each DNA profile with a ranking value. In some embodiments, the ranking value is determined based on the number of related DNA profiles in the full database that is ranked from least to most, and ties are broken by the number of ancestry-diverged DNA profiles in the full database as ranked from most to least. In some embodiments, step (3) involves going iteratively through the ranked DNA profiles, and for each DNA profile: (i) if the DNA profile is not yet in the related sample set, adding it to the unrelated sample set and adding all related DNA profiles to the related sample set, and (ii) if the DNA profile is already in the related sample set, then skipping to the next DNA profile, and repeating beginning at step (3) (i).

In some of any of such embodiments, the calculating the degree of relationship comprises calculating a kinship coefficient using PC-Relate. In some of any of such embodiments, the degree of relationship is calculated by providing the DNA profile of the person of interest as input to PC-Relate. In some of any of such embodiments, the degree of relationship is calculated by providing the kinship model and the DNA profile of the person of interest as input to PC-Relate. In some of any of such embodiments, the one or more reference DNA profiles are further provided as input to PC-Relate.

In some of any of such embodiments, the calculating the degree of relationship comprises calculating a kinship coefficient using a whole genome kinship algorithm as follows:

$$\text{?} = \frac{{\sum }_{s ∈ S} ( {g}_{is} - 2 {\hat{u}}_{is} ) ( {g}_{js} - 2 {\hat{u}}_{js} )}{4 {{\sum }_{s ∈ S} [ {\hat{u}}_{is} ( 1 - {\hat{u}}_{is} ) \cdot {\hat{u}}_{js} ( 1 - {\hat{u}}_{js} ) ]}^{1 / 2}}$$

wherein the person of interest and a reference DNA profile of the one or more reference DNA profiles are i and j, [symbol] is the kinship coefficient, û is the estimated allele frequencies, s is a SNP in S SNPs that were typed in both individuals, gᵢₛ and gⱼₛ are the number of reference alleles in i and j at SNP s, respectively, and ûᵢₛ and ûⱼₛ are the expected allele frequencies calculated by PC-AiR for i and j at SNP s, respectively.

In some of any of such embodiments, the calculating the degree of relationship comprises calculating a likelihood ratio. In some embodiments, the calculating the likelihood ratio comprises comparing the plurality of SNPs between the DNA profile and the one or more reference DNA profiles. In some embodiments, the calculating the likelihood ratio comprises comparing a set of SNPs comprising kinship SNPs from within the plurality of SNPs between the DNA profile and the one or more reference DNA profiles.

In some of any of such embodiments, calculating the likelihood ratio comprises dividing the probability of the DNA profile and a reference DNA profile from among the one or more reference DNA profiles being related by the probability of the DNA profile and the reference DNA profile being unrelated based on the genotypes of the plurality of SNPs.

In some of any of such embodiments, the likelihood ratio (LR) is calculated as follows:

$$LR = \frac{P ( D \vert {H}_{r} )}{P ( D \vert {H}_{u} )}$$

wherein D represents the genotypes, Hᵣ represents the hypothesis that the individuals are related, and Hᵤ represents the hypothesis that the individuals are unrelated.

In some of any of such embodiments, the LR is calculated as as follows:

$$LR = \frac{0.001}{{p}^{2} {q}^{2}}$$

wherein 0.001 represents a genotyping error rate, p is the allele frequency of the allele 1, and q is the allele frequency of allele 2.

In some of any of such embodiments, the person of interest is biologically male and the method further comprises calculating a likelihood ratio for sharing a Y chromosome between the DNA profile and the one or more reference DNA profiles. In some embodiments, the calculating a likelihood ratio for sharing a Y chromosome comprises comparing a set of SNPs that comprises one or more Y-SNPs between the DNA profile and the one or more reference DNA profiles.

In some of any of such embodiments, the one or more Y-SNPs are comprised within the plurality of SNPs. In some embodiments, the one or more Y-SNPs comprises at least 20, 25, 30, 35, 40, 45, 50, 55, 60, 65, 70, 75, 80, 81, 82, 83, 84, or 85 Y-SNPs. In some of any of such embodiments, the one or more Y-SNPs comprises 85 Y-SNPs.

In some of any of such embodiments, calculating the likelihood ratio for sharing a Y chromosome comprises dividing the probability of the DNA profile and a reference DNA profile from among the one or more reference DNA profiles sharing a Y chromosome by the probability of the DNA profile and the reference DNA profile not sharing a Y chromosome based on the genotypes of the one or more Y-SNPs.

In some of any of such embodiments, at least 90%, 91%, 92%, 93%, 94%, 95%, 96%, 97%, 98%, or 99% of the DNA profiles in the reference set of DNA profiles is from a relative of a missing person or a victim of a disaster or a conflict. In some of any of such embodiments, each of the DNA profiles in the reference set of DNA profiles is from a relative of a missing person or a victim of a disaster or a conflict.

In some of any of such embodiments, the reference set of DNA profiles comprises up to 5, 10, 15, 20, 25, 30, 35, 40, 45, 50, 55, 60, 65, 70, 75, 80, 85, 90, 95, 100, 125, 150, 175, 200, 300, 400, 500, 600, 700, 800, 900, or 1000 reference DNA profiles. In some of any of such embodiments, the reference set of DNA profiles comprises up to 100 reference DNA profiles.

In some of any of such embodiments, at least 5%, 10%, 15%, 20%, 25%, 30%, 35%, 40%, 45%, 50%, 55%, 60%, 65%, 70%, 75%, 80%, 85%, 90%, or 95% of the reference DNA profiles in the reference set of DNA profiles is from a relative of the person of interest. In some of any of such embodiments, at least 50% of the reference DNA profiles in the reference set of DNA profiles is from a relative of the person of interest.

In some of any of such embodiments, each relative of the person of interest in the reference set of DNA profiles is individually a first degree, second degree, third degree, fourth degree, or fifth degree relative of the person of interest.

In some of any of such embodiments, the identity of each relative of the person of interest in the reference set of DNA profiles is known. In some of any of such embodiments, the identity of each relative of the person of interest in the reference set of DNA profiles is known. In some of any of such embodiments, the identity of each of the one or more reference DNA profiles in the reference set of DNA profiles is known.

In some of any of such embodiments, the reference set of DNA profiles is in a database. In some embodiments, the database is not publicly accessible. In some of any of such embodiments, the database is not accessible by a third party geneaological service.

Also provided herein is a nucleic acid library constructed using any of the methods described herein.

Also provided herein is a plurality of primers that specifically hybridize to a plurality of target sequences comprising at least between at or about 2,000 to 50,000 single nucleotide polymorphisms (SNPs) in a nucleic acid sample from a person of interest, wherein amplifying the nucleic acid sample using the plurality of primers in one or more multiplex PCR reactions results in amplification products.

Also provided herein is a plurality of primers that specifically hybridize to a plurality of target sequences comprising at least between at or about 2,000 to 50,000 single nucleotide polymorphisms (SNPs) in a nucleic acid sample from a person of interest and a nucleic acid sample from one or more reference samples, wherein the one or more reference samples comprises a sample from a relative of the person of interest, and wherein amplifying the nucleic acid sample from the person of interest and the nucleic acid sample from one or more reference samples using the plurality of primers in one or more multiplex PCR reactions results in amplification products.

In some of any of such embodiments, the nucleic acid sample from the person of interest comprises genomic DNA.

In some of any of such embodiments, the nucleic acid sample from the person of interest comprises one or more enzyme inhibitors. In some of any of such embodiments, the one or more enzyme inhibitors comprise one or more inhibitors selected from the group consisting of hematin, heme, humic acid, indigo, tannic acid, collagen, calcium, and hydroxyapatite. In some of any of such embodiments, the nucleic acid sample from the person of interest comprises low-quality nucleic acid molecules and/or low quantity nucleic acid molecules. In some embodiments, the low quality nucleic acid molecules are degraded genomic DNA and/or fragmented genomic DNA. In some of any of such embodiments, the low quality nucleic acid molecules have a degradation index (DI) of at or at least 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200. In some of any of such embodiments, the low quality nucleic acid molecules have a DI of at least 1 and up to or less than 158.3. In some of any of such embodiments, the nucleic acid sample from the person of interest and/or the nucleic acid sample from one or more reference samples comprises high quality nucleic acid molecules. In some of any of such embodiments, the high quality nucleic acid molecules have a DI of less than 1.

In some of any of such embodiments, the person of interest is a missing person. In some of any of such embodiments, the person of interest is a victim of a disaster or conflict.

In some of any of such embodiments, the nucleic acid sample from the person of interest is derived from a buccal swab, paper, fabric, or other substrate or object that is impregnated with saliva, blood, or other bodily fluid, or contains hair or skin cells.

In some of any of such embodiments, the nucleic acid sample from the person of interest comprises between or between about 3 pg and 100 ng of genomic DNA. In some of any of such embodiments, the nucleic acid sample from the person of interest comprises between or between about 100 pg and 5 ng of genomic DNA, between or between about 50 pg and 5 ng of genomic DNA, or between or between about 3 pg and 5 ng of genomic DNA. In some of any of such embodiments, the nucleic acid sample from the person of interest comprises at or about 1 ng of genomic DNA.

In some of any of such embodiments, the plurality of SNPs comprises kinship SNPs (kiSNPs). In some of any of such embodiments, the plurality of SNPs comprises Y-chromosome SNPs (Y-SNPs). In some of any of such embodiments, the plurality of SNPs comprises kiSNPs and Y-SNPs. In some of any of such embodiments, the plurality of SNPs comprises kiSNPs, biogeographical ancestry SNPs (aiSNPs), identity SNPs (iiSNPs), phenotype SNPs (piSNPs), X-chromosome SNPs (X-SNPs), and Y-chromosome SNPs (Y-SNPs). In some of any of such embodiments, the plurality of SNPs comprises SNPs selected from one or more of the groups consisting of kiSNPs, aiSNPs, iiSNPs, piSNPs, X-SNPs, and Y-SNPs.

In some of any of such embodiments, at least or at least about 80%, 81%, 82%, 83%, 84%, 85%, 86%, 87%, 88%, 89%, 90%, 91%, 92%, 93%, 94%, 95%, 96%, 97%, 98%, or 99% of the plurality of SNPs are kinship SNPs. In some of any of such embodiments, at least 90%, 91%, 92%, 93%, 94%, 95%, 96%, 97%, 98%, or 99% of the DNA profiles in the reference set of DNA profiles is from a relative of a missing person or a victim of a disaster or a conflict.

In some of any of such embodiments, each of the one or more references samples is from a relative of a missing person or a victim of a disaster or a conflict. In some of any of such embodiments, the one or more reference samples comprises up to 5, 10, 15, 20, 25, 30, 35, 40, 45, 50, 55, 60, 65, 70, 75, 80, 85, 90, 95, 100, 125, 150, 175, 200, 300, 400, 500, 600, 700, 800, 900, or 1000 reference samples. In some of any of such embodiments, at least 5%, 10%, 15%, 20%, 25%, 30%, 35%, 40%, 45%, 50%, 55%, 60%, 65%, 70%, 75%, 80%, 85%, 90%, or 95% of the one or more reference samples is from a relative of the person of interest. In some of any of such embodiments, at least 5%, 10%, 15%, 20%, 25%, 30%, 35%, 40%, 45%, 50%, 55%, 60%, 65%, 70%, 75%, 80%, 85%, 90%, or 95% of the reference DNA profiles in the reference set of DNA profiles is from a relative of the person of interest, and wherein each of the at least 5%, 10%, 15%, 20%, 25%, 30%, 35%, 40%, 45%, 50%, 55%, 60%, 65%, 70%, 75%, 80%, 85%, 90%, or 95% of the reference DNA profiles in the reference set of DNA profiles is a first degree, second degree, third degree, fourth degree, or fifth degree relative. In some of any of such embodiments, at least 50% of the one or more reference samples is from a relative of the person of interest. In some of any of such embodiments, each relative of the person of interest in the one or more reference samples is individually a first degree, second degree, third degree, fourth degree, or fifth degree relative of the person of interest. In some of any of such embodiments, each relative of the person of interest in the one or more reference samples is individually a first degree, second degree, or third degree relative of the person of interest. In some of any of such embodiments, the identity of each relative of the person of interest in the one or more reference samples is known. In some of any of such embodiments, the identity of each of the one or more reference samples is known.

Also provided herein is a method for constructing a DNA profile, comprising: providing a nucleic acid sample from a person of interest, amplifying the nucleic acid sample with a plurality of primers that specifically hybridize to a plurality of target sequences collectively comprising a plurality of at least between at or about 2,000 to 50,000 single nucleotide polymorphisms (SNPs), thereby generating amplification products, wherein the amplification is carried out in one or more multiplex PCR reactions, sequencing the amplification products, and determining the genotypes of the plurality of SNPs, thereby generating a DNA profile.

Also provided herein is a method for constructing a DNA profile, comprising: providing a nucleic acid sample from a person of interest, providing a nucleic acid sample from a relative of the person of interest, amplifying the nucleic acid sample from the person of interest and the nucleic acid sample from the relative with a plurality of primers that specifically hybridize to a plurality of target sequences collectively comprising a plurality of at least between at or about 2,000 to 50,000 single nucleotide polymorphisms (SNPs), thereby generating amplification products, wherein the amplification is carried out in one or more multiplex PCR reactions, sequencing the amplification products, and determining the genotypes of the plurality of SNPs, thereby generating a DNA profile for the person of interest and the relative of the person of interest.

In some of any of such embodiments, the sequencing does not comprise whole genome sequencing (WGS). In some of any of such embodiments, the nucleic acid sample comprises genomic DNA. In some of any of such embodiments, the nucleic acid sample of the person of interest and/or the nucleic acid sample of the relative of the person of interest comprises genomic DNA.

In some of any of such embodiments, the nucleic acid sample, the nucleic acid sample of the person of interest, and/or the nucleic acid sample of the relative comprises one or more enzyme inhibitors. In some embodiments, the one or more enzyme inhibitors comprise one or more inhibitors selected from the group consisting of hematin, heme, humic acid, indigo, tannic acid, collagen, calcium, and hydroxyapatite.

In some of any of such embodiments, the nucleic acid sample, the nucleic acid sample of the person of interest, and/or the nucleic acid sample of the relative comprises low-quality nucleic acid molecules and/or low quantity nucleic acid molecules. In some embodiments, the low quality nucleic acid molecules are degraded genomic DNA and/or fragmented genomic DNA. In some of any of such embodiments, the low quality nucleic acid molecules have a degradation index (DI) of at or at least 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200. In some of any of such embodiments, the low quality nucleic acid molecules have a DI of at least 1 and up to or less than 158.3. In some of any of such embodiments, the nucleic acid sample, the nucleic acid sample of the person of interest, and/or the nucleic acid sample of the relative comprises high quality nucleic acid molecules. In some embodiments, the high quality nucleic acid molecules have a DI of less than 1.

In some of any of such embodiments, the person of interest is a missing person. In some of any of such embodiments, the person of interest is a victim of a disaster or conflict.

In some of any of such embodiments, the relative of the person of interest is a first-, second-, third-, fourth-, or fifth-degree relative. In some of any of such embodiments, the relative of the person of interest is a first-, second-, or third-degree relative.

In some of any of such embodiments, the nucleic acid sample, the nucleic acid sample of the person of interest, and/or the nucleic acid sample of the relative is derived from a buccal swab, paper, fabric, or other substrate or object that is impregnated with saliva, blood, or other bodily fluid, or contains hair or skin cells.

In some of any of such embodiments, the nucleic acid sample, the nucleic acid sample of the person of interest, and/or the nucleic acid sample of the relative comprises between or between about 3 μg and 100 ng of genomic DNA. In some of any of such embodiments, the nucleic acid sample, the nucleic acid sample of the person of interest, and/or the nucleic acid sample of the relative comprises between or between about 100 pg and 5 ng of genomic DNA, between or between about 50 pg and 5 ng of genomic DNA, or between or between about 3 pg and 5 ng of genomic DNA. In some of any of such embodiments, the nucleic acid sample, the nucleic acid sample of the person of interest, and/or the nucleic acid sample of the relative comprises at or about 1 ng of genomic DNA.

In some of any of such embodiments, the plurality of SNPs comprises kinship SNPs. In some of any of such embodiments, the plurality of SNPs comprises Y-chromosome SNPs (Y-SNPs). In some of any of such embodiments, the plurality of SNPs comprises kiSNPs and Y-SNPs. In some of any of such embodiments, the plurality of SNPs comprises kiSNPs, biogeographical ancestry SNPs (aiSNPs), identity SNPs (iiSNPs), phenotype SNPs (piSNPs), X-chromosome SNPs (X-SNPs), and Y-chromosome SNPs (Y-SNPs). In some of any of such embodiments, the plurality of SNPs comprises SNPs selected from one or more of the groups consisting of kiSNPs, aiSNPs, iiSNPs, piSNPs, X-SNPs, and Y-SNPs. In some of any of such embodiments, at least or at least about 80%, 81%, 82%, 83%, 84%, 85%, 86%, 87%, 88%, 89%, 90%, 91%, 92%, 93%, 94%, 95%, 96%, 97%, 98%, or 99% of the plurality of SNPs are kinship SNPs.

In some of any of such embodiments, the sequencing comprises a sequencing plexity of up to 40-plex. In some of any of such embodiments, the sequencing comprises a sequencing plexity of up to 32-plex. In some of any of such embodiments, the sequencing comprises a sequencing plexity of 12-plex to 32-plex. In some of any of such embodiments, the sequencing comprises a sequencing plexity of 24-plex to 32-plex. In some of any of such embodiments, the sequencing comprises a sequencing plexity of at or about 4-plex, 5-plex, 6-plex, 7-plex, 8-plex, 9-plex, 10-plex, 11-plex, 12-plex, 13-plex, 14-plex, 15-plex, 16-plex, 17-plex 18-plex, 19-plex, 20-plex, 21-plex, 22-plex, 23-plex, 24-plex, 25-plex, 26-plex, 27-plex, 28-plex, 29-plex, 30-plex, 31-plex, 32-plex, 33-plex, 34-plex, 35-plex, 36-plex, 37-plex, 38-plex, 39-plex, 40-plex, 41-plex, 42-plex, 43-plex, 44-plex, or 45-plex. In some of any of such embodiments, the sequencing comprises a sequencing plexity of at or about 10-plex, 11-plex, 12-plex, 13-plex, 14-plex, 15-plex, 16-plex, 17-plex 18-plex, 19-plex, 20-plex, 21-plex, 22-plex, 23-plex, 24-plex, 25-plex, 26-plex, 27-plex, 28-plex, 29-plex, 30-plex, 31-plex, 32-plex, 33-plex, 34-plex, or 35-plex. In some of any of such embodiments, the sequencing comprises a sequencing plexity of at or about 8-to 16-plex for postmortem samples, and/or the sequencing comprises a sequencing plexity of at or about 24-to 40-plex for antemortem samples. In some of any of such embodiments, the sequencing comprises a sequencing plexity of at or about 12-plex for postmortem samples, and/or the sequencing comprises a sequencing plexity of at or about 32-plex for antemortem samples. In some of any of such embodiments, the sequencing comprises a sequencing plexity of at or about 30-plex, 31-plex, or 32-plex.

Also provided herein is a method of identifying genetic relatives of a DNA profile, comprising: calculating the degree of relationship of the DNA profile of any one of claims 127-161 to one or more reference DNA profiles, wherein the one or more reference DNA profiles are comprised within a reference set of DNA profiles comprising one or more reference DNA profiles from a relative of the person of interest; and generating a family tree comprising the DNA profile in relation to the one or more reference DNA profiles.

In some embodiments, the one or more reference DNA profiles are part of a database.

In some of any of such embodiments, the reference set of DNA profiles comprises up to 5, 10, 15, 20, 25, 30, 35, 40, 45, 50, 55, 60, 65, 70, 75, 80, 85, 90, 95, 100, 125, 150, 175, 200, 300, 400, 500, 600, 700, 800, 900, or 1000 reference DNA profiles. In some of any of such embodiments, at least 5%, 10%, 15%, 20%, 25%, 30%, 35%, 40%, 45%, 50%, 55%, 60%, 65%, 70%, 75%, 80%, 85%, 90%, or 95% of the reference DNA profiles in the reference set of DNA profiles is from a relative of the person of interest. In some of any of such embodiments, at least 50% of the reference DNA profiles in the reference set of DNA profiles is from a relative of the person of interest. In some of any of such embodiments, each relative of the person of interest is individually a first degree, second degree, third degree, fourth degree, or fifth degree relative of the person of interest.

In some of any of such embodiments, the identity of each relative of the person of interest in the reference set of DNA profiles is known. In some of any of such embodiments, the identity of each of the one or more reference DNA profiles in the reference set of DNA profiles is known.

In some of any of such embodiments, the reference set of DNA profiles is in a database. In some embodiments, the database is not publicly accessible. In some of any of such embodiments, the database is not accessible by a third party geneaological service.

Also provided herein is a method of identifying the identity of a DNA profile, comprising: calculating the degree of relationship of a DNA profile comprising genotypes of at least between at or about 2,000 to 50,000 SNPs to one or more reference DNA profiles, wherein the DNA profile is from a person of interest, and wherein the one or more reference DNA profiles are comprised within a reference set of DNA profiles comprising one or more reference DNA profiles from a relative of the person of interest; and generating a family tree comprising the DNA profile in relation to the one or more reference DNA profiles.

In some embodiments, the DNA profile is generated by any of the methods for generating a DNA profile described herein.

In some of any of such embodiments, the degree of relationship is calculated using a kinship model. In some of any of such embodiments, the degree of relationship is calculated using a kinship model that is trained using a PCA method. In some embodiments, the PCA method for training the kinship model is PCA or involves PCA. In some of any of such embodiments, the PCA method is PC-AiR. In some of any of such embodiments, the PC-AiR comprises the steps of: (1) estimating kinship coefficients between all pairs of samples, optionally training DNA profiles, of a training database, wherein pairings with a kinship coefficient >0.025 are identified as related and pairings with a kinship coefficient <−0.025 are identified as ancestry-diverged; (2) initializing an unrelated sample set that includes all samples; and (3) iteratively: (i) identifying the set in the unrelated sample set that have the most related samples in the unrelated sample set, thereby designated as X, (ii) identifying the set of samples in X that have the least ancestry-diverged pairings compared to samples in the unrelated sample set, thereby designated as Y; and (iii) if Y has zero samples, then terminating the process, or, if Y has at least one sample, then randomly selecting one sample from Y to remove from U, and repeat beginning at step (3) (i).

In some of any of such embodiments, the PCA method is a modified PC-Air. In some embodiments, the modified PC-AiR comprises steps of: (1) estimating kinship coefficients between all pairs of samples, optionally training DNA profiles, of a training database, wherein pairings with a kinship coefficient >0.01 are identified as related and pairings with a kinship coefficient <−0.025 are identified as ancestry-diverged; (2) removing all DNA profiles that have ≥5% missing data; and (3) ranking all DNA profiles by identifying each DNA profile with a ranking value. In some embodiments, the ranking value is determined based on the number of related DNA profiles in the full database that is ranked from least to most, and ties are broken by the number of ancestry-diverged DNA profiles in the full database as ranked from most to least. In some embodiments, step (3) involves going iteratively through the ranked DNA profiles, and for each DNA profile: (i) if the DNA profile is not yet in the related sample set, adding it to the unrelated sample set and adding all related DNA profiles to the related sample set, and (ii) if the DNA profile is already in the related sample set, then skipping to the next DNA profile, and repeating beginning at step (3) (i).

In some of any of such embodiments, the calculating the degree of relationship comprises calculating a kinship coefficient using PC-Relate. In some embodiments, the degree of relationship is calculated by providing the DNA profile of the person of interest as input to PC-Relate. In some of any of such embodiments, the degree of relationship is calculated by providing the kinship model and the DNA profile of the person of interest as input to PC-Relate.

In some of any of such embodiments, the one or more reference DNA profiles are further provided as input to PC-Relate.

In some of any of such embodiments, the calculating the degree of relationship comprises calculating a kinship coefficient using a whole genome kinship algorithm as follows:

$$\text{?} = \frac{{\sum }_{s ∈ S} ( {g}_{is} - 2 {\hat{u}}_{is} ) ( {g}_{js} - 2 {\hat{u}}_{js} )}{4 {{\sum }_{s ∈ S} [ {\hat{u}}_{is} ( 1 - {\hat{u}}_{is} ) \cdot {\hat{u}}_{js} ( 1 - {\hat{u}}_{js} ) ]}^{1 / 2}}$$

wherein the person of interest and a reference DNA profile of the one or more reference DNA profiles are i and j, [symbol] is the kinship coefficient, û is the estimated allele frequencies, s is a SNP in S SNPs that were typed in both individuals, gᵢₛ and gⱼₛ are the number of reference alleles in i and j at SNP s, respectively, and ûᵢₛ and ûⱼₛ are the expected allele frequencies calculated by PC-AiR for i and j at SNP s, respectively.

In some of any of such embodiments, the calculating the degree of relationship comprises calculating a likelihood ratio. In some embodiments, the calculating the likelihood ratio comprises comparing the plurality of SNPs between the DNA profile and the one or more reference DNA profiles. In some embodiments, the calculating the likelihood ratio comprises comparing a set of SNPs comprising kinship SNPs from within the plurality of SNPs between the DNA profile and the one or more reference DNA profiles.

In some of any of such embodiments, calculating the likelihood ratio comprises dividing the probability of the DNA profile and a reference DNA profile from among the one or more reference DNA profiles being related by the probability of the DNA profile and the reference DNA profile being unrelated based on the genotypes of the plurality of SNPs.

In some of any of such embodiments, the likelihood ratio (LR) is calculated as follows:

$$LR = \frac{P ( D \vert {H}_{r} )}{P ( D \vert {H}_{u} )}$$

wherein D represents the genotypes, Hᵣ represents the hypothesis that the individuals are related, and Hᵤ represents the hypothesis that the individuals are unrelated.

In some of any of such embodiments, the LR is calculated as as follows:

$$LR = \frac{0.001}{{p}^{2} {q}^{2}}$$

wherein 0.001 represents a genotyping error rate, p is the allele frequency of the allele 1, and q is the allele frequency of allele 2.

In some of any of such embodiments, the person of interest is biologically male and the method further comprises calculating a likelihood ratio for sharing a Y chromosome between the DNA profile and the one or more reference DNA profiles. In some embodiments, the calculating a likelihood ratio for sharing a Y chromosome comprises comparing a set of SNPs that comprises one or more Y-SNPs between the DNA profile and the one or more reference DNA profiles. In some embodiments, the one or more Y-SNPs are comprised within the plurality of SNPs. In some of any of such embodiments, the one or more Y-SNPs comprises at least 20, 25, 30, 35, 40, 45, 50, 55, 60, 65, 70, 75, 80, 81, 82, 83, 84, or 85 Y-SNPs. In some of any of such embodiments, the one or more Y-SNPs comprises 85 Y-SNPs.

In some of any of such embodiments, calculating the likelihood ratio for sharing a Y chromosome comprises dividing the probability of the DNA profile and a reference DNA profile from among the one or more reference DNA profiles sharing a Y chromosome by the probability of the DNA profile and the reference DNA profile not sharing a Y chromosome based on the genotypes of the one or more Y-SNPs.

In some of any of such embodiments, at least 90%, 91%, 92%, 93%, 94%, 95%, 96%, 97%, 98%, or 99% of the DNA profiles in the reference set of DNA profiles is from a relative of a missing person or a victim of a disaster or a conflict.

In some of any of such embodiments, each of the one or more references samples is from a relative of a missing person or a victim of a disaster or a conflict. In some of any of such embodiments, the one or more reference samples comprises up to 5, 10, 15, 20, 25, 30, 35, 40, 45, 50, 55, 60, 65, 70, 75, 80, 85, 90, 95, 100, 125, 150, 175, 200, 300, 400, 500, 600, 700, 800, 900, or 1000 reference samples.

In some of any of such embodiments, at least 5%, 10%, 15%, 20%, 25%, 30%, 35%, 40%, 45%, 50%, 55%, 60%, 65%, 70%, 75%, 80%, 85%, 90%, or 95% of the one or more reference samples is from a relative of the person of interest. In some of any of such embodiments, at least 5%, 10%, 15%, 20%, 25%, 30%, 35%, 40%, 45%, 50%, 55%, 60%, 65%, 70%, 75%, 80%, 85%, 90%, or 95% of the reference DNA profiles in the reference set of DNA profiles is from a relative of the person of interest, and wherein each of the at least 5%, 10%, 15%, 20%, 25%, 30%, 35%, 40%, 45%, 50%, 55%, 60%, 65%, 70%, 75%, 80%, 85%, 90%, or 95% of the reference DNA profiles in the reference set of DNA profiles is a first degree, second degree, third degree, fourth degree, or fifth degree relative. In some of any of such embodiments, at least 50% of the one or more reference samples is from a relative of the person of interest. In some of any of such embodiments, each relative of the person of interest in the one or more reference samples is individually a first degree, second degree, third degree, fourth degree, or fifth degree relative of the person of interest. In some of any of such embodiments, each relative of the person of interest in the one or more reference samples is individually a first degree, second degree, or third degree relative of the person of interest.

In some of any of such embodiments, the identity of each relative of the person of interest in the one or more reference samples is known. In some of any of such embodiments, the identity of each of the one or more reference samples is known.

Also provided herein is a kit comprising at least one container means, wherein the at least one container means comprises any of the plurality of primers described herein. In some of any of such embodiments, the plurality of SNPs comprises between or between about 2,000 to 11,000 SNPs, 3,000 to 11,000 SNPs, 4,000 to 11,000 SNPs, 5,000 to 11,000 SNPs, 5,500 to 11,000 SNPs, 6,000 to 11,000 SNPs, 7,000 to 15,000 SNPs, 7,000 to 14,000 SNPs, 7,000 to 13,000 SNPs, 7,000 to 12,000 SNPs, 7,000 to 11,000 SNPs, 8,000 to 15,000 SNPs, 8,000 to 14,000 SNPs, 8,000 to 13,000 SNPs, 8,000 to 12,000 SNPs, 8,000 to 11,000 SNPs, 9,000 to 15,000 SNPs, 9,000 to 14,000 SNPs, 9,000 to 13,000 SNPs, 9,000 to 12,000 SNPs, or 9,000 to 11,000 SNPs. In some of any of such embodiments, the plurality of SNPs comprises 10,230 SNPs. In some of any of such embodiments, the plurality of SNPs comprises between or between about 2,000 to 11,000 SNPs, 3,000 to 11,000 SNPs, 4,000 to 11,000 SNPs, 5,000 to 11,000 SNPs, 5,500 to 11,000 SNPs, 6,000 to 11,000 SNPs, 7,000 to 15,000 SNPs, 7,000 to 14,000 SNPs, 7,000 to 13,000 SNPs, 7,000 to 12,000 SNPs, 7,000 to 11,000 SNPs, 8,000 to 15,000 SNPs, 8,000 to 14,000 SNPs, 8,000 to 13,000 SNPs, 8,000 to 12,000 SNPs, 8,000 to 11,000 SNPs, 9,000 to 15,000 SNPs, 9,000 to 14,000 SNPs, 9,000 to 13,000 SNPs, 9,000 to 12,000 SNPs, or 9,000 to 11,000 SNPs. In some of any of such embodiments, the plurality of SNPs comprises 10,230 SNPs.

In some of any of such embodiments, the method further comprises generating a family tree comprising the DNA profile in relation to one or more DNA profiles comprised in the reference set of DNA profiles. In some embodiments, the family tree comprises the DNA profile in relation to one or more DNA profiles from a relative of the person of interest.

### BRIEF DESCRIPTION OF THE DRAWINGS

FIG. 1 depicts an exemplary schematic of a method of generating a library capable of being sequenced.

FIG. 2 shows the results of the number of loci identified using varying input titrations of genomic DNA including 5 ng, 2.5 ng, 1 ng, 500 pg, 250 pg, 100 pg and 50 pg.

FIG. 3 shows the percentage of Loci detected (call rate) with degraded DNA using the assay described herein compared to Microarray (GSA) call rate.

FIG. 4 shows the number of loci detected in the presence of inhibitors hematin, humic acid, indigo, and tannic acid, compared to a reference control.

FIG. 5 depicts an exemplary family tree generated by the methods described herein.

FIG. 6 shows the expected and observed Kinship Coefficients calculated using the algorithm described herein.

FIG. 7 shows the results of the 1: many search algorithm in an exemplary case study.

FIG. 8. depicts an exemplary family tree generated from the results of the 1: many search algorithm.

FIG. 9 is a table summarizing the number and type of loci detected using varying input titrations of genomic DNA, including 5 ng, 2.5 ng, 1 ng, 500 pg, 250 pg, 100 pg, and 50 pg.

FIG. 10 is a table summarizing the number and type of loci detected using DNA in the presence of the inhibitors hematin, humic acid, tannic acid, and indigo, compared to a positive amplification control, in the absence of inhibitors.

FIG. 11 is a table summarizing the number and type of loci detected for two samples of DNA obtained 9 hours and 22 hours after a mock sexual assault. The DNA was isolated from the sperm fraction of a differential extraction method, and had an input of 500 pg of DNA.

FIG. 12 shows the number of loci detected in saliva samples with an increasing content of phenol (a known PCR amplification inhibitor) from a phenol-chloroform-isoamyl alcohol (PCIA) extraction method.

FIG. 13 shows the number of loci detected in blood samples isolated from different substrates or methods typically performed in forensics laboratories, including blood with rust, blood in denim, blood on a swab, and blood with varying levels of heme (a known PCR amplification inhibitor) carry-over from Chelex™ extraction.

FIG. 14 depicts an exemplary schematic of a method of evaluating kinship for individuals of interest, e.g., missing persons or victims of conflicts or disasters, that includes analyzing kinship using DNA profiles, e.g., SNP reports, uploaded to a local server that includes at least one DNA profile from a relative.

FIG. 15 shows the total number of SNPs detected for individual samples within an exemplary set of true postmortem samples.

FIG. 16 shows the total number of SNPs detected for individual samples within an exemplary related mock postmortem sample set that included samples that were artificially degraded by boiling or were low input samples having a DI of 0 and less than 1 ng of input DNA.

FIG. 17 shows the total number of SNPs detected for individual samples within an exemplary related antemortem private family sample set that included samples from CEPH/Utah that include up to second degree relationships as verified at Coriell, as well as three unrelated samples.

FIG. 18A-E depicts receiver operating characteristic (ROC) curves of the results for 2,000 SNPs (FIG. 18A), 4,000 SNPs (FIG. 18B), 6,000 SNPs (FIG. 18C), 8,000 SNPs (FIG. 18D), and 10,000 SNPs (FIG. 18E) on first degree, second degree, and third degree relatives.

FIG. 19A-E depicts ROC curves of the results for 2,000 SNPs (FIG. 19A), 4,000 SNPs (FIG. 19B), 6,000 SNPs (FIG. 19C), 8,000 SNPs (FIG. 19D), and 10,000 SNPs (FIG. 19E) on fourth or fifth degree relatives.

FIG. 20A depicts the distribution of the number of SNPs typed in a downsampled dataset of sequenced libraries at 16-plex and 30-plex. FIGS. 20B and 20C depict the number of SNPs typed in sequence library samples sequenced in a 3 (3plex), 12 (12plex), 16 (16plex), 24 (24plex), and 32 (32plex) sample runs for mock antemortem (AM) samples whose libraries were generated with 1 ng intact DNA (FIG. 20B), and in 3 (3plex) and 12 (12plex) sample runs for mock postmortem (PM) samples whose libraries were generated from cremated, interred, and burnt bones, dental remains, degraded DNA samples from whole blood, and low input DNA samples of 50, 100, 250, and 500 pg input DNA (FIG. 20C).

FIGS. 21A and 21B depict allele concordance and heterozygosity of mock antemortem (mock AM) samples (FIG. 21A) and mock postmortem (mock PM) (FIG. 21B) samples from contemporary teeth, blood, buried bone, contemporary bone, or low input DNA.

FIGS. 22A-D depict graphical representations of the sensitivity and specificity of whole kinship coefficients for anonymized GEDMatch samples, for first-degree, second-degree, third-degree, fourth-degree, and fifth-degree relationships, in which the number of SNPs typed are 2,000 (FIG. 22A), 4,000 (FIG. 22B), 6,000 (FIG. 23C), or 8,000 (FIG. 22D).

FIG. 23 depicts a pedigree of the Utah/CEPH 1463 family that consists of grandparents, parents, and sublings, thereby representing first and second-degree relationships. Samples were sequenced at 12, 16, 24, and 32 sample pooled libraries.

FIG. 24 depicts the distribution of the number of SNPs typed across samples in the Utah/CEPH 1463 family when sequenced at four different numbers of samples per run: 12 samples per run (12plex), 16 samples per run (16plex), 24 samples per run (24plex), and 32 samples per run (32plex).

FIGS. 25A and 25B depict the distribution of the kinship coefficient (FIG. 25A) and the log base 10 likelihood ratio (Log LR) (FIG. 25B) for all combinations of pairs taken from the Utah/CEPH 1463 family and 100 randomly selected samples from the 1000 Genomes Project, which represented unrelated controls. The samples include samples from grandparents (G), parents (P), siblings(S), unrelated controls (U), unrelated grandparents (GU), unrelated parents (PU), and unrelated siblings (SU).

FIG. 26 depicts a pedigree of a private related family (RF) that consists of parents, an aunt, a first cousin (Cousin), a first cousin once removed (1C1R), and a second cousin.

FIG. 27A depicts the distribution of the number of SNPs typed for the private related family (RF) that inced first-, second-, third-, fourth-, and fifth-degree relationships, using degraded/low input DNA samples at 12plex, and intact samples at 30plex. FIG. 27B depicts kinship coefficients of pairs of individuals from the private related family (RF), with corresponding log likelihood ratios indicated on the top of each bar.

### DETAILED DESCRIPTION

The practice of the techniques described herein may employ, unless otherwise indicated, conventional techniques and descriptions of molecular biology, cell biology, biochemistry and sequencing technology, which are within the skill of those who practice in the art. Specific illustrations of suitable techniques can be had by reference to the examples herein.

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### OVERVIEW

Samples from missing persons or victims of disasters or conflicts can be highly degraded and may not be suitable for whole genome sequencing (WGS), microarray, or short tandem repeat (STR) analysis. Mitochondrial analysis has high sensitivity and may be suitable in certain situations, but only considers the maternal line of inheritance, thereby having drawbacks for use in kinship analysis. Moreover, current methods of generating DNA profiles for comparisons in genetic databases include genotyping using dense SNP microarrays and WGS followed by association of evidentiary samples with distant relatives in databases, which require high quantity and high quality DNA samples, and are not designed for familial searching or for use in identifying missing persons or victims of disasters or conflicts. These samples can be low quantity and low quality samples, e.g., includes degraded DNA, and data from the current methods requires extensive imputation to generate results capable of being uploaded to a search database. Finally, relatives of missing persons or victims of disasters or conflicts often do not want to have their genetic data uploaded to public databases. The new and improved methods provided herein overcome these limitations by allowing for the use of low quantity and low quality, e.g., degraded, DNA for the generation of nucleic acid profiles, for a more efficient genetic analysis than alternative approaches like WGS or SNP microarrays, and without needing to upload genetic data into a publicly accessible genetic database. Moreover, the new and improved methods provided herein also include an improved method of performing kinship analysis that requires fewer computations for calculating accurate kinship.

Identification of victims of fatality incidents is required for both humanitarian and legal reasons: giving families searching for missing family members resolution and justice for families when death is not accidental as in civil and criminal cases. Identification of victims of mass fatality incidents (MFI) can be challenging given the large numbers of victims and the impact of the disaster on the integrity of the bodies of the victims. MFI are caused by accidents, such as disease/famine, earthquakes/tsunamis/hurricanes, plane/train/automobile crashes, or fires, or by human intent, such as war, terrorist attacks, or human rights violations/genocide. Recent events such as the terrorist attack of the World Trade Center in New York City of 11 Sep. 2001 or the boxing day tsunami in of 26 Dec. 2004 caused by the earthquake off the west coast of northern Sumatra caused unimaginable loss of human life and illustrated the need for efficient procedures and methods for recovering and cataloging the remains of the victims, storing information related to the remains, and identification.

The most common methods for identification (referred to as disaster victim identification or DVI) are fingerprinting, dental comparisons (dental examinations or radiology), itemization of personal effects, autopsy examination for evidence of surgical scars/procedures and tattoos, and analysis of DNA. Traditional methods such as fingerprinting and dental comparisons are often utilized as a first course of action due to the low labor and cost required as well as the speed of the procedures, but these methods require pre-mortem records of fingerprints and dental images for the identification. Personal effects can be misleading as victims may have similar jewelry or other personal items. Comparison of surgical procedures and tattoos also require pre-mortem medical records or documentation of tattoos or other alterations. Lastly, these methods require that the remains of the victims are relatively intact. Some MFI can result in fragmentation of the remains as well as comingling. In the cases of fragmentation, DNA analysis of the postmortem (PM) samples can not only assist with the identification of the missing person but assist with assignment of multiple remains or body parts to a specific individual. DNA analysis requires antemortem (AM) samples such as razors, shavers, toothbrushes, or hairbrushes from the missing person for comparison. When AM samples from the missing person are not available, samples donated from close family members will assist with identification. DNA analysis is more time consuming than traditional methods and has specific requirements for laboratory cleanliness and tracking chain of custody for the samples to be analyzed which can be challenging in field situations.

In the cases where traditional methods are unsuccessful or the remains are not intact, successful DNA analysis will depend on the collection of the samples, the timing of that collection, storage conditions and the amount of the samples obtained (relevant in cases with decomposition and putrefaction). DNA identification relies on non-coding DNA markers used in forensic genomics including short tandem repeats (STRs), such as the set of 20 autosomal core loci included in the Combined DNA Index System or CODIS, mitochondrial DNA for maternal lineage, or STRs on the Y chromosome (Y-STRs) for paternal lineage. Autosomal markers are preferrable due to the complication that multiple family members can be missing in the mass disaster sharing either the same maternal or paternal lineage.

STR analysis has been used successfully for many years for human identification in criminal, missing persons, and paternity cases. The success of this type of analysis is the result of the highly polymorphic nature of these markers and the number of markers that can be multiplexed together for one analysis. These markers have also been utilized successfully for DVI. In the cases of MFI, a software solution is helpful for assisting with the large numbers of pair-wise comparisons of profiles from PM (victim) and AM (self or relative) samples and statistical calculations for the degree of relatedness. Several software packages are available for analysis of autosomal and Y-STRs as well as mitochondrial DNA data. Not all samples from MFI are amenable to STR analysis. Highly degraded DNA samples do not amplify the larger STR markers in the commonly used capillary electrophoresis-based (CE) kits limiting the number of markers typed to make an identification. Development of CE kits utilizing smaller amplicons has assisted with CE analysis of degraded DNA (Butler paper). Next generation sequencing (NGS or massively parallel sequencing) STR assays allow for smaller amplicon sizes as well as analyzing more markers within one assay which both improve recovery of information from degraded DNA samples.

Utilizing STR data is especially appropriate for cases where AM DNA samples are available from the missing person or very close family members such as first-degree relatives (parent, child or sibling). STR analysis is less successful if only more distant family members are available for comparisons due to the number of false positive identifications that can occur with unrelated individuals. This is especially true in the cases where the MFI occurred many years in the past and very close family members from the missing are deceased. In cases where DNA from more distant order relatives (second-and third-degree relatives such as nieces, nephews, grandchildren or great grandchildren) is available for comparisons, utilizing a larger number of markers such as single nucleotide polymorphisms (SNPs) can assist with identification.

The methods described herein were developed to interrogate 10,230 forensically-relevant SNPs for purpose such as solving cold cases, including missing persons identification. Previous approaches involved analyzing amplified DNA utilizing NGS for up to 3 samples per sequencing run. Such an approach was initially designed to type sufficient SNPs for detecting relationships up to fifth-degree when searching DNA microarray databases, such as GEDmatch PRO to assist law enforcement in solving cold cases. To facilitate DVI, requiring a higher throughput solution that is cost effective, a new and improved method was developed where amplified DNA can be sequenced at higher plexities to type sufficient SNPs with significant overlap to determine relationships to third-degree. As shown in Example 13, mock PM samples sequenced at a multiplexity of 12 sample per run and mock AM samples sequenced at a multiplexity of 32 samples per run generated enough typed loci data to identify relationships out to third-degree with no false positive identifications using the methods and kinship algorithm described herein.

The goal of this kinship algorithm was three-fold: to identify relationships in the absence of a pedigree or information of a relationship, which can be important when trying to assign multiple remains to a single individual or when a relative of a victim is not available or known; to reduce the false positive rate in identifying relationships, which is often elevated when calculating likelihood ratios using STRs (Alonso et al., Croat Med J 2005, 46, 540-548, the content of which is hereby incorporated by reference in its entirety); and, maintain privacy for victims of MFIs, which requires knowledge of a programming language such as R, a local build of a likelihood ratio software like Familias (Kling et al., Forensic Sci Int Genet 2014, 13, 121-127, doi: 10.1016/j.fsigen.2014.07.004, the content of which is hereby incorporated by reference in its entirety), or a private account on Bonaparte (Slooten et al., Forensic Science International: Genetics 2011, 5, 308-315, doi: 10.1016/j.fsigen.2010.06.005, the content of which is hereby incorporated by reference in its entirety) (can only identify up to second-degree relationships). Since victims of MFIs and victims' families often request privacy in identification of remains, this requires maintenance of genotype information on a private server. As such, in some embodiments, the kinship algorithm described herein is localized on a private server to identify relationships among samples prepared as described herein. The local software did not upload results to a law enforcement database; rather, maintained results for review on the private server. Additionally, in some embodiments, the kinship algorithm described herein can efficiently identify relationships, with perfect sensitivity and specificity, up to and including third-degree for degraded/low input mock PM samples sequenced at a plexity of 12 and for mock reference or AM samples sequenced at a plexity of 32.

Accordingly, disclosed herein are methods of identifying a person of interest by performing a DNA-based kinship analysis using a DNA profile from the person of interest, e.g., a missing person or a victim of a disaster or conflict, and determining the degree of relationship between that DNA profile and one or more reference DNA profiles that includes known relatives of the person of interest, thereby identifying the person of interest.

Provided herein is a method for performing DNA-based kinship analysis, comprising: providing a nucleic acid sample from a person of interest, amplifying the nucleic acid sample with a plurality of primers that specifically hybridize to a plurality of target sequences collectively comprising a plurality of at least between at or about 2,000 to 50,000 single nucleotide polymorphisms (SNPs), thereby generating amplification products, wherein the amplification is carried out in one or more multiplex PCR reactions, generating a nucleic acid library from the amplification products, sequencing the nucleic acid library generated from the amplification products, analyzing the sequences of the amplification products, determining the genotypes of the plurality of SNPs, thereby generating a DNA profile, and calculating the degree of relationship of the DNA profile to one or more reference DNA profiles, wherein the one or more reference DNA profiles are comprised within a reference set of DNA profiles comprising one or more reference DNA profiles from a relative of the person of interest.

Also provided herein is a method for performing DNA-based kinship analysis, comprising: providing a nucleic acid sample from a person of interest, amplifying the nucleic acid sample with a plurality of primers that specifically hybridize to a plurality of target sequences collectively comprising a plurality of at least between at or about 2,000 to 50,000 single nucleotide polymorphisms (SNPs), thereby generating amplification products, wherein the amplification is carried out in one or more multiplex PCR reactions, generating a nucleic acid library from the amplification products sequencing the nucleic acid library generated from the amplification products, determining the genotypes of the plurality of SNPs, thereby generating a DNA profile, and calculating the degree of relationship of the DNA profile to one or more reference DNA profiles, wherein the one or more reference DNA profiles are comprised within a reference set of DNA profiles comprising one or more reference DNA profiles from a relative of the person of interest.

Also provided herein is a method of constructing a nucleic acid library of a person of interest, comprising: providing a nucleic acid sample from a person of interest, amplifying the nucleic acid sample with a plurality of primers that specifically hybridize to a plurality of target sequences collectively comprising a plurality of at least between at or about 2,000 to 50,000 single nucleotide polymorphisms (SNPs), thereby generating a nucleic acid library comprising amplification products, wherein the amplification is carried out in one or more multiplex PCR reactions.

Also provided herein is a method of constructing a nucleic acid library for a reference DNA sample, comprising: providing a nucleic acid sample from a relative of a person of interest, amplifying the nucleic acid sample with a plurality of primers that specifically hybridize to a plurality of target sequences collectively comprising a plurality of at least between at or about 2,000 to 50,000 single nucleotide polymorphisms (SNPs), thereby generating a nucleic acid library comprising amplification products, wherein the amplification is carried out in one or more multiplex PCR reactions. In some embodiments, the relative is a first-, second-, third-, fourth-, or fifth-degree relative of the person of interest. In some embodiments, the relative is a first-, second-, or third-degree relative of the person of interest.

Also provided herein is a method for calculating degree of relatedness, comprising: obtaining a DNA profile comprising genotypes of at least between at or about 2,000 to 50,000 SNPs, wherein the DNA profile is from a person of interest; and calculating the degree of relationship of the DNA profile to one or more reference DNA profiles, wherein the one or more reference DNA profiles are comprised within a reference set of DNA profiles comprising one or more reference DNA profiles from a relative of the person of interest.

Also provided herein is a method for calculating degree of relatedness, comprising: generating a DNA profile comprising genotypes of at least between at or about 2,000 to 50,000 SNPs, wherein the DNA profile is from a person of interest; and calculating the degree of relationship of the DNA profile to one or more reference DNA profiles, wherein the one or more reference DNA profiles are comprised within a reference set of DNA profiles comprising one or more reference DNA profiles from a relative of the person of interest.

Also provided herein is a nucleic acid library constructed using any of the methods described herein, e.g., any of the methods for constructing a nucleic acid library as described herein.

Also provided herein is a plurality of primers that specifically hybridize to a plurality of target sequences comprising at least between at or about 2,000 to 50,000 single nucleotide polymorphisms (SNPs) in a nucleic acid sample from a person of interest, wherein amplifying the nucleic acid sample using the plurality of primers in one or more multiplex PCR reactions results in amplification products.

Also provided herein is a plurality of primers that specifically hybridize to a plurality of target sequences comprising at least between at or about 2,000 to 50,000 single nucleotide polymorphisms (SNPs) in a nucleic acid sample from a person of interest and a nucleic acid sample from one or more reference samples, wherein the one or more reference samples comprises a sample from a relative of the person of interest, and wherein amplifying the nucleic acid sample from the person of interest and the nucleic acid sample from one or more reference samples using the plurality of primers in one or more multiplex PCR reactions results in amplification products.

Also provided herein is a method for constructing a DNA profile, comprising: providing a nucleic acid sample from a person of interest, amplifying the nucleic acid sample with a plurality of primers that specifically hybridize to a plurality of target sequences collectively comprising a plurality of at least between at or about 2,000 to 50,000 single nucleotide polymorphisms (SNPs), thereby generating amplification products, wherein the amplification is carried out in one or more multiplex PCR reactions, sequencing the amplification products, determining the genotypes of the plurality of SNPs, thereby generating a DNA profile.

Also provided herein is a method for constructing a DNA profile, comprising: providing a nucleic acid sample from a person of interest, providing a nucleic acid sample from a relative of the person of interest, amplifying the nucleic acid sample from the person of interest and the nucleic acid sample from the relative with a plurality of primers that specifically hybridize to a plurality of target sequences collectively comprising a plurality of at least between at or about 2,000 to 50,000 single nucleotide polymorphisms (SNPs), thereby generating amplification products, wherein the amplification is carried out in one or more multiplex PCR reactions, sequencing the amplification products, determining the genotypes of the plurality of SNPs, thereby generating a DNA profile for the person of interest and the relative of the person of interest.

Also provided herein is a DNA profile constructed using any of the methods as described herein, e.g., any of the methods for constructing a DNA profile as described herein.

Also provided herein is a method of identifying genetic relatives of a DNA profile, comprising: calculating the degree of relationship of any of the DNA profiles as described herein to one or more reference DNA profiles, wherein the one or more reference DNA profiles are comprised within a reference set of DNA profiles comprising one or more reference DNA profiles from a relative of the person of interest; and generating a family tree comprising the DNA profile in relation to the one or more reference DNA profiles.

Also provided herein is a method of identifying the identity of a DNA profile, comprising: calculating the degree of relationship of a DNA profile comprising genotypes of at least between at or about 2,000 to 50,000 SNPs to one or more reference DNA profiles, wherein the DNA profile is from a person of interest, and wherein the one or more reference DNA profiles are comprised within a reference set of DNA profiles comprising one or more reference DNA profiles from a relative of the person of interest; and generating a family tree comprising the DNA profile in relation to the one or more reference DNA profiles.

Also provided herein is a kit comprising at least one container means, wherein the at least one container means comprises any of the plurality of primers described herein.

In some of any of such embodiments, the degree of relationship is calculated using a kinship model. In some of any of such embodiments, the degree of relationship is calculated using a kinship model that is trained using a PCA method. In some of any of such embodiments, the PCA method for training the kinship model is PCA or involves PCA.

In some of any of such embodiments, the PCA method is PC-AiR. In some embodiments, the PC-AiR comprises the steps of: (1) estimating kinship coefficients between all pairs of samples, optionally training DNA profiles, of a training database, wherein pairings with a kinship coefficient >0.025 are identified as related and pairings with a kinship coefficient <−0.025 are identified as ancestry-diverged; (2) initializing an unrelated sample set that includes all samples; and (3) iteratively: (i) identifying the set in the unrelated sample set that have the most related samples in the unrelated sample set, thereby designated as X, (ii) identifying the set of samples in X that have the least ancestry-diverged pairings compared to samples in the unrelated sample set, thereby designated as Y; and (iii) if Y has zero samples, then terminating the process, or, if Y has at least one sample, then randomly selecting one sample from Y to remove from U, and repeat beginning at step (3) (i).

In some of any of such embodiments, the PCA method is a modified PC-Air. In some embodiments, the modified PC-AiR comprises steps of: (1) estimating kinship coefficients between all pairs of samples, optionally training DNA profiles, of a training database, wherein pairings with a kinship coefficient >0.01 are identified as related and pairings with a kinship coefficient <−0.025 are identified as ancestry-diverged; (2) removing all DNA profiles that have ≥5% missing data; and (3) ranking all DNA profiles by identifying each DNA profile with a ranking value. In some embodiments, the ranking value is determined based on the number of related DNA profiles in the full database that is ranked from least to most, and ties are broken by the number of ancestry-diverged DNA profiles in the full database as ranked from most to least. In some embodiments, step (3) involves going iteratively through the ranked DNA profiles, and for each DNA profile: (i) if the DNA profile is not yet in the related sample set, adding it to the unrelated sample set and adding all related DNA profiles to the related sample set, and (ii) if the DNA profile is already in the related sample set, then skipping to the next DNA profile, and repeating beginning at step (3) (i).

In some of any of such embodiments, the calculating the degree of relationship comprises calculating a kinship coefficient using PC-Relate. In some embodiments, the degree of relationship is calculated by providing the DNA profile of the person of interest as input to PC-Relate. In some of any of such embodiments, the degree of relationship is calculated by providing the kinship model and the DNA profile of the person of interest as input to PC-Relate. In some of any of such embodiments, the one or more reference DNA profiles are further provided as input to PC-Relate. In some of any of such embodiments, the calculating the degree of relationship comprises calculating a likelihood ratio. In some embodiments, the calculating the likelihood ratio comprises comparing the plurality of SNPs between the DNA profile and the one or more reference DNA profiles. In some embodiments, the calculating the likelihood ratio comprises comparing a set of SNPs comprising kinship SNPs from within the plurality of SNPs between the DNA profile and the one or more reference DNA profiles. In some embodiments, the person of interest is biologically male and the method further comprises calculating a likelihood ratio for sharing a Y chromosome between the DNA profile and the one or more reference DNA profiles. In some embodiments, the calculating a likelihood ratio for sharing a Y chromosome comprises comparing a set of SNPs that comprises one or more Y-SNPs between the DNA profile and the one or more reference DNA profiles. In some embodiments, the one or more Y-SNPs are comprised within the plurality of SNPs. In some embodiments, the one or more Y-SNPs comprises at least 20, 25, 30, 35, 40, 45, 50, 55, 60, 65, 70, 75, 80, 81, 82, 83, 84, or 85 Y-SNPs. In some embodiments, the one or more Y-SNPs comprises 85 Y-SNPs. In some embodiments, calculating the likelihood ratio for sharing a Y chromosome comprises dividing the probability of the DNA profile and a reference DNA profile from among the one or more reference DNA profiles sharing a Y chromosome by the probability of the DNA profile and the reference DNA profile not sharing a Y chromosome based on the genotypes of the one or more Y-SNPs.

In some embodiments, calculating the likelihood ratio comprises dividing the probability of the DNA profile and a reference DNA profile from among the one or more reference DNA profiles being related by the probability of the DNA profile and the reference DNA profile being unrelated based on the genotypes of the plurality of SNPs.

In some of any of such embodiments, the calculating the likelihood of sharing chromosome Y comprises calculating a log likelihood by providing the DNA profile of the person of interest and a matching profile as input to identify matching chromosomes.

### Samples and Sample Processing

In some aspects, the sample disclosed herein can be or comprise any suitable biological sample, or a sample derived therefrom. In some aspects, the samples described herein are processed and amplified using any known suitable method to complement the methods described herein. Exemplary samples, methods of sample processing and methods of sample amplification are described below.

### A. Nucleic Acid Samples

A nucleic acid sample disclosed herein can be derived from any biological sample, e.g., any biological sample from a person of interest. A biological sample may be derived from blood, buccal swabs, hair, teeth, bone, skin, tissue, and/or semen, or any other source for obtaining DNA of the person of interest. In some embodiments, the nucleic acid sample is derived from a biological sample that is or comprises blood, hair, teeth, bone, semen, skin, or sperm. In some embodiments, the nucleic acid sample is derived from a tissue sample. In some embodiments, the biological sample is a DNA sample. In some embodiments, the nucleic acid sample comprises DNA. In some embodiments, the DNA is genomic DNA (gDNA). In some embodiments, the nucleic acid sample from the person of interest comprises genomic DNA and/or the nucleic acid sample from a reference DNA sample, e.g., a relative of the person of interest, comprises genomic DNA. In some embodiments, the nucleic acid sample of the person of interest and/or the nucleic acid sample of the relative of the person of interest comprises genomic DNA. The DNA from which the nucleic acid sample may be obtained may be intact or partially degraded. The DNA from which the nucleic acid sample may be obtained may be compromised, degraded or inhibited due, but not limited to, to source material age, variable extraction, storage procedures or environmental exposure. In some embodiments, the DNA is compromised due to calcium inhibition, cremation, burning, and embalming. In some embodiments, the methods described herein comprise providing a nucleic acid sample from a person of interest.

In some embodiments, the DNA from which the nucleic acid sample is obtained is a low quantity and/or low quality DNA sample. In some embodiments, the DNA from which the nucleic acid sample is obtained is a low quantity and low quality DNA sample. In some embodiments, the low quality DNA sample comprises low quality nucleic acid molecules. In some embodiments, the low quality nucleic acid molecules are degraded DNA, e.g., genomic DNA, and/or are fragmented DNA, e.g., genomic DNA.

The quality of a nucleic acid, e.g., DNA, sample can be determined by calculating a degradation index (DI). DI is calculated by dividing the concentration of small DNA targets by the concentration of large DNA targets (DI=concentration of small DNA targets/concentration of large DNA targets). In general, a DI value of less than 1 typically indicates that the nucleic acid, e.g., DNA, is not degraded, is not a low quality sample, and/or is a high quality sample; a DI value of 1 to 10 typically indicates that the nucleic acid, e.g., DNA, has a minor to moderate amount of degradation; and a DI value of greater than 10 typically indicates that the nucleic acid, e.g., DNA, is highly degraded.

In some embodiments, the low quality nucleic acid molecules have a DI of at or at least 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 or more. In some embodiments, the low quality nucleic acid molecules have a DI of at least 1 and at or less than 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 or more. In some embodiments, the low quality nucleic acid molecules have a DI of at least 2 and at or less than 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 or more. In some embodiments, the low quality nucleic acid molecules have a DI of at or at least 2 or more. In some embodiments, the low quality nucleic acid molecules have a DI of at or at least 5 or more. In some embodiments, the low quality nucleic acid molecules have a DI of at or at least 10 or more. In some embodiments, the low quality nucleic acid molecules have a DI of at or at least 20 or more. In some embodiments, the low quality nucleic acid molecules have a DI of between 1 and 200. In some embodiments, the low quality nucleic acid molecules have a DI of between 1 and 175. In some embodiments, the low quality nucleic acid molecules have a DI of at least 1 and at or less than 158.3. In some embodiments, the low quality nucleic acid molecules have a DI of between 2 and 200. In some embodiments, the low quality nucleic acid molecules have a DI of between 2 and 175. In some embodiments, the low quality nucleic acid molecules have a DI of at least 2 and at or less than 158.3. In some embodiments, the low quality nucleic acid molecules have a DI of between 5 and 200. In some embodiments, the low quality nucleic acid molecules have a DI of between 5 and 175. In some embodiments, the low quality nucleic acid molecules have a DI of at least 5 and at or less than 158.3. In some embodiments, the low quality nucleic acid molecules have a DI of between 10 and 200. In some embodiments, the low quality nucleic acid molecules have a DI of between 10 and 175. In some embodiments, the low quality nucleic acid molecules have a DI of at least 10 and at or less than 158.3.

In some embodiments, the low quality nucleic acid molecules have a DI of between or between about 1 and 10, between or between about 1 and 50, between or between about 1 and 100, between or between about 1 and 200, between or between about 2 and 10, between or between about 2 and 50, between or between about 2 and 100, between or between about 200, between or between about 5 and 10, between or between about 5 and 50, between or between about 5 and 100, between or between about 5 and 200.

In some embodiments, the DNA from which the nucleic acid sample is obtained is a high quality nucleic acid sample. In some embodiments, the high quality nucleic acid sample has a DI of less than 1.

In some embodiments, the nucleic acid sample comprises one or more enzyme inhibitors. In some embodiments, the one or more enzyme inhibitors comprise one or more inhibitors selected from the group consisting of hematin, humic acid (e.g., heme), humic acid, indigo, tannic acid, collagen, calcium, and hydroxyapatite. In some embodiments, the one or more enzyme inhibitors comprises heme.

In some embodiments, the nucleic acid sample is from a person of interest, e.g., a missing person or a victim of a disaster or conflict. In some embodiments, the person of interest is a missing person. A missing person may be missing for any reason, and may be missing voluntarily or involuntarily. For instance, in some embodiments, the missing person is missing involuntarily, and has been abducted or kidnapped. In some embodiments, the missing person is missing voluntarily, and had run away, is evading detection, or is otherwise in hiding.

In some embodiments, the nucleic acid sample is from a reference DNA sample. In some embodiments, the reference DNA sample is from a relative of the person of interest. Accordingly, in some embodiments, the nucleic acid sample is from a relative of the person of interest, such as a first-, second-, third-, fourth-, or fifth-degree relative of the person of interest. In some embodiments, one or more of the one or more reference DNA profiles is derived from a reference DNA sample, e.g., a reference DNA sample from a relative of the person of interest.

In some embodiments, the person of interest is a victim of a disaster or conflict. A victim of a disaster or conflict may be a victim of any type of disaster or conflict. For instance, in some embodiments, the victim of a disaster or conflict is a victim of a disaster, such as a hurricane, a tornado, a storm, a fire, including a wildfire/forest fire, a tsunami, an earthquake, a flood, a volcanic eruption, an avalanche, and the like. In some embodiments, the disaster is a natural disaster. As used herein, “natural disaster” refers to any disaster resulting from natural processes of the Earth, such as related to weather and/or geological events, e.g., hurricanes, floods, storms, tsunamis, earthquakes, volcanic eruptions, etc. In some embodiments, the disaster is a non-natural disaster. As used herein, “non-natural disaster” refers to any disaster other than a natural disaster, including those resulting from human influence, including disasters involving automotive vehicles, planes, ships, and trains, disasters involving the collapse of building, roads, mines, and bridges, disasters involving burning buildings, among other disasters resulting from human influence. In some embodiments, the victim of a disaster or conflict is a victim of a conflict, such as a war or other conflict among groups of people. As used herein, “conflict” refers to any conflict, e.g., an armed conflict, between different nations or states or different groups within a nation or state, e.g., war, or a terrorist attack, or any other conflict between groups that results in human death and/or injury.

In some embodiments, the person of interest is biologically female. In some embodiments, the person of interest is biologically male.

In some embodiments, the nucleic acid sample is derived from a buccal swab, paper, fabric, e.g., denim, or other substrate or object that is impregnated with saliva, blood, sperm, or other bodily fluid, or contains hair or skin cells. In some embodiments, the object that is impregnated with saliva, blood, sperm, or other bodily fluid or contains hair or skin cells is a personal object, such as a toothbrush or a hairbrush. In some embodiments, the nucleic acid sample is derived from an object that contains hair or skin cells, e.g., a hairbrush or a toothbrush. In some embodiments, the nucleic acid sample is derived from a personal object, e.g., a toothbrush or a hairbrush. In some embodiments, the nucleic acid sample is derived from a toothbrush or a hairbrush. In some embodiments, the personal object is an object that is used by, and/or associated with, the person from which the nucleic acid sample is derived, such that the person's nucleic acids are present on or in the object.

In some embodiments, the nucleic acid sample is from a crime scene, such as a homicide, an assault, such as a sexual assault, or a burglary, or any other crime where identification of a participant is needed. In some embodiments, the nucleic acid sample is from a sexual assault.

In some embodiments, the nucleic acid sample is obtained at or about 30 minutes, at or about 1 hour, or at or about 2, 3, 4, 5, 6, 7, 8, 9, 10, 11, 12, 13, 14, 15, 16, 17, 18, 19, 20, 21, 22, 23, or 24 or more hours after a sample containing the nucleic acid sample was deposited by its source, e.g., a human subject. In some embodiments, the nucleic acid sample is obtained at or less than about 3 hours, 9 hours, 12 hours, 15 hours, 18 hours, 21 hours, 22 hours, 24 hours, 36 hours, 48 hours, 3 days, 4 days, 5 days, 6 days, 7 days, 2 weeks, 3 weeks, 4 weeks, 1 month, 2 months, 3 months, 4 months, 5 months, 6 months, 7 months, 8 months, 9 months, 10 months, 11 months, 1 year, 2 years, 3 years, or 4 or more years after a sample containing the nucleic acid sample was deposited by its source, e.g., a human subject. In some embodiments, the nucleic acid sample is obtained at or less than 24 hours, e.g., at or less than 22 hours, after a sample containing the nucleic acid sample was deposited by its source, e.g., a human subject.

In some embodiments, the nucleic acid sample comprises between or between about 3 pg and 100 ng of DNA, e.g., genomic DNA, or between or between about 50 pg and 100 ng of DNA, e.g., genomic DNA. In some embodiments, the nucleic acid sample comprises between or between about 100 μg and 5 ng of DNA, e.g., genomic DNA. In some embodiments, the nucleic acid sample comprises at or about 1 ng of DNA, e.g., genomic DNA. In some embodiments, the nucleic acid sample comprises between or between about 3 pg and 100 ng of DNA, e.g., genomic DNA.

In some embodiments, the nucleic acid sample comprises at or about 3 pg to at or about 100 ng of DNA, e.g., genomic DNA. In some embodiments, the nucleic acid sample comprises at or about 10 μg to at or about 100 ng of DNA, e.g., genomic DNA, or comprises at or about 10 pg to at or about 5 ng of DNA, e.g., genomic DNA. In some embodiments, the nucleic acid sample comprises at or about 10 pg to 10 ng, at or about 10 pg to 5 ng, at or about 25 pg to 10 ng, at or about 25 pg to 5 ng, at or about 50 pg to 10 ng, or at or about 50 pg to 5 ng, of DNA, e.g., genomic DNA.

In some embodiments, the nucleic acid sample comprises at or about 3 pg to at or about 5 ng of DNA, e.g., genomic DNA. In some embodiments, the nucleic acid sample comprises at or about 50 pg to at or about 5 ng of DNA, e.g., genomic DNA. In some embodiments, the nucleic acid sample comprises at or about 2.5 ng, 3 pg, 4 pg, 5 pg, 6 pg, 7 pg, 8 pg, 9 pg, 10 pg, 15 pg, 20 pg, 25 pg, 30 pg, 35 pg, 40 pg, 45 pg, 50 pg, 55 pg, 60 pg, 70 pg, 75 pg, 80 pg, 85 pg, 90 pg, 95 pg, 100 pg, 125 pg, 150 pg, 175 pg, 200 pg, 225 pg, 250 pg, 275 pg, 300 pg, 325 pg, 350 pg, 375 pg, 400 pg, 420 pg, 425 pg, 450 pg, 475 pg, 500 pg, 600 pg, 700 pg, 800 pg, 900 pg, 1 ng, 1.1 ng, 1.2 ng, 1.3 ng, 1.4 ng, 1.5 ng, 1.6 ng, 1.7 ng, 1.8 ng, 1.9 ng, 2 ng, 2.1 ng, 2.2 ng, 2.3 ng, 2.4 ng, 2.5 ng, 2.6 ng, 2.7 ng, 2.8 ng, 2.9 ng, 3 ng, 3.25 ng, 3.5 ng, 3.75 ng, 4 ng, 4.25 ng, 4.5 ng, 4.75 ng, or 5 ng of DNA, e.g., genomic DNA, or between any two preceding values. In some embodiments, the nucleic acid sample comprises between or between about 3 μg and 10 ng, between or between about 3 pg and 5 ng, between or between about 3 pg and 4 ng, between or between about 3 pg and 3 ng, between or between about 3 pg and 2 ng, between or between about 10 pg and 10 ng, between or between about 10 pg and 5 ng, between or between about 10 pg and 4 ng, between or between about 10 pg and 3 ng, between or between about 10 pg and 2 ng, between or between about 25 pg and 10 ng, between or between about 25 pg and 5 ng, between or between about 25 μg and 4 ng, between or between about 25 pg and 3 ng, between or between about 25 pg and 2 ng, between or between about 40 pg and 10 ng, between or between about 40 pg and 5 ng, between or between about 40 pg and 4 ng, between or between about 40 pg and 3 ng, between or between about 40 μg and 2 ng, between or between about 50 pg and 10 ng, between or between about 50 pg and 5 ng, between or between about 50 pg and 4 ng, between or between about 50 pg and 3 ng, between or between about 50 pg and 2 ng, between or between about 10 pg and 2 ng, between or between about 10 pg and 1.5 ng, between or between about 10 pg and 1 ng, between or between about 20 pg and 2 ng, between or between about 20 pg and 1.5 ng, between or between about 20 pg and 1 ng, 25 pg and 2 ng, between or between about 25 pg and 1.5 ng, between or between about 25 pg and 1 ng, between or between about 30 μg and 2 ng, between or between about 30 pg and 1.5 ng, between or between about 30 pg and 1 ng, between or between about 35 pg and 2 ng, between or between about 35 pg and 1.5 ng, between or between about 35 pg and 1 ng, between or between about 40 pg and 2 ng, between or between about 40 μg and 1.5 ng, between or between about 40 pg and 1 ng, between or between about 45 pg and 2 ng, between or between about 45 pg and 1.5 ng, between or between about 45 pg and 1 ng, between or between about 50 pg and 2 ng, between or between about 50 pg and 1.5 ng, or between or between about 50 pg and 1 ng.

### B. Sample Processing and Amplification

In some embodiments, the methods provided herein comprise a step of amplifying the nucleic acid sample with a plurality of primers that specifically hybridize to a plurality of target sequences collectively comprising a plurality of at least between at or about 2,000 to 50,000 single nucleotide polymorphisms (SNPs), thereby generating amplification products, wherein the amplification is carried out in one or more multiplex PCR reactions.

A variety of steps can be performed to prepare or process a nucleic acid sample for and/or during an assay. Except where indicated otherwise, the preparative or processing steps described below can generally be combined in any manner and in any order to appropriately prepare or process a particular sample for analysis and/or sequencing, disclosed herein.

In some embodiments, the amount of the nucleic acid sample provided is, is about, or is less than 1 ng of genomic DNA. In some embodiments, the methods disclosed herein comprise amplification of the genomic DNA. In some embodiments, amplification of the genomic DNA includes one or more multiplex polymerase chain reactions (PCR) comprising a plurality of primers, thereby generating amplification products. In some embodiments, amplification of the genomic DNA includes a single multiplex PCR reaction. In some embodiments, amplification of the genomic DNA includes two multiplex PCR reactions. In some embodiments, amplification of the genomic DNA includes three multiplex PCR reactions. In some embodiments, amplification of the genomic DNA includes four multiplex PCR reactions.

In some embodiments, one or more primers in the plurality of primers are designed in accordance with the atypical design strategy as described in WO 2015/126766 A1, which is hereby incorporated by reference in its entirety. In some embodiments, one or more primers in the plurality of primers is at least 24 nucleotides in length, and/or has a melting temperature that is less than 60 degrees C, and/or is AT-rich with an AT content of at least 60%. In some embodiments, one or more primers in the plurality of primers comprises a length of at least 24 nucleotides that hybridize to the target sequence, and/or has a melting temperature that is between 50 degrees C and 60 degrees C, and/or is AT-rich with an AT content of at least 60%. In some embodiments, one or more primers in the plurality of primers has a melting temperature that is less than 58 degrees C, or is less than 54 degrees C.

In some embodiments, the genomic DNA may be amplified for a number of cycles using the plurality of primers that hybridize and/or tag a plurality of target sequences collectively comprising a plurality of at least between at or about 2,000 to 50,000 single nucleotide polymorphisms (SNPs), or at least between at or about 5,000 to 50,000. In some embodiments, the genomic DNA may be amplified for a number of cycles using the plurality of primers that hybridize and/or tag a plurality of target sequences collectively comprising at least between at or about 2,000 to 15,000, 20,000, 25,000, 30,000, 35,000, 40,000, 45,000, or 50,000 SNPs. In some embodiments, the genomic DNA may be amplified for a number of cycles using the plurality of primers that hybridize and/or tag a plurality of target sequences collectively comprising at least between at or about 5,000 to 15,000, 20,000, 25,000, 30,000, 35,000, 40,000, 45,000, or 50,000 SNPs. In some embodiments, the genomic DNA may be amplified for a number of cycles using the plurality of primers that hybridize and/or tag a plurality of target sequences collectively comprising at least between at or about 10,000 to 11,000 SNPs. In some embodiments, the genomic DNA may be amplified for a number of cycles using the plurality of primers that hybridize and/or tag a plurality of target sequences collectively comprising at least between at or about 2,000 to 11,000 SNPs, 3,000 to 11,000 SNPs, 4,000 to 11,000 SNPs, 5,000 to 11,000 SNPs, 5,500 to 11,000 SNPs, 6,000 to 11,000 SNPs, 7,000 to 15,000 SNPs, 7,000 to 14,000 SNPs, 7,000 to 13,000 SNPs, 7,000 to 12,000 SNPs, 7,000 to 11,000 SNPs, 8,000 to 15,000 SNPs, 8,000 to 14,000 SNPs, 8,000 to 13,000 SNPs, 8,000 to 12,000 SNPs, 8,000 to 11,000 SNPs, 9,000 to 15,000 SNPs, 9,000 to 14,000 SNPs, 9,000 to 13,000 SNPs, 9,000 to 12,000 SNPs, or 9,000 to 11,000 SNPs. In some embodiments, the genomic DNA may be amplified for a number of cycles using the plurality of primers that hybridize and/or tag a plurality of target sequences collectively comprising at least between at or about 6,000 to 11,000 SNPs. In some embodiments, the plurality of SNPs comprises at or about 2,639 SNPs. In some embodiments, the genomic DNA may be amplified for a number of cycles using the plurality of primers that hybridize and/or tag a plurality of target sequences collectively comprising at or about 10,230 SNPs.

In some embodiments, the plurality of SNPs comprises at least between at or about 2,000 to 15,000, 20,000, 25,000, 30,000, 35,000, 40,000, 45,000, or 50,000 SNPs. In some embodiments, the plurality of SNPs comprises at least between at or about 5,000 to 15,000, 20,000, 25,000, 30,000, 35,000, 40,000, 45,000, or 50,000 SNPs. In some embodiments, the plurality of SNPs comprises at least between at or about 6,000 to 15,000, 20,000, 25,000, 30,000, 35,000, 40,000, 45,000, or 50,000 SNPs. In some embodiments, the plurality of SNPs comprises at least between at or about 2,000 to 11,000 SNPs, 3,000 to 11,000 SNPs, 4,000 to 11,000 SNPs, 5,000 to 11,000 SNPs, 5,500 to 11,000 SNPs, 6,000 to 11,000 SNPs, 7,000 to 15,000 SNPs, 7,000 to 14,000 SNPs, 7,000 to 13,000 SNPs, 7,000 to 12,000 SNPs, 7,000 to 11,000 SNPs, 8,000 to 15,000 SNPs, 8,000 to 14,000 SNPs, 8,000 to 13,000 SNPs, 8,000 to 12,000 SNPs, 8,000 to 11,000 SNPs, 9,000 to 15,000 SNPs, 9,000 to 14,000 SNPs, 9,000 to 13,000 SNPs, 9,000 to 12,000 SNPs, or 9,000 to 11,000 SNPs. In some embodiments, the plurality of SNPs comprises at or about 2,639 SNPs. In some embodiments, the plurality of SNPs comprises at or about 10,230 SNPs. In some embodiments, the plurality of SNPs comprises at least between at or about 2,000 to 50,000 SNPs, 5,000 to 50,000 SNPs, 5,000 to 45,000 SNPs, 5,000 to 40,000 SNPs, 5,000 to 35,000 SNPs, 5,000 to 30,000 SNPs, 5,000 to 25,000 SNPs, 5,000 to 20,000 SNPs, 6,000 to 50,000 SNPs, 6,000 to 45,000 SNPs, 6,000 to 40,000 SNPs, 6,000 to 35,000 SNPs, 6,000 to 30,000 SNPs, 6,000 to 25,000 SNPs, 6,000 to 20,000 SNPs, 7,000 to 50,000 SNPs, 7,000 to 45,000 SNPs, 7,000 to 40,000 SNPs, 7,000 to 35,000 SNPs, 7,000 to 30,000 SNPs, 7,000 to 25,000 SNPs, 7,000 to 20,000 SNPs, 8,000 to 50,000 SNPs, 8,000 to 45,000 SNPs, 8,000 to 40,000 SNPs, 8,000 to 35,000 SNPs, 8,000 to 30,000 SNPs, 8,000 to 25,000 SNPs, 8,000 to 20,000 SNPs, 9,000 to 50,000 SNPs, 9,000 to 45,000 SNPs, 9,000 to 40,000 SNPs, 9,000 to 35,000 SNPs, 9,000 to 30,000 SNPs, 9,000 to 25,000 SNPs, or 9,000 to 20,000 SNPs.

In some embodiments, the plurality of SNPs comprises at least between at or about 2,000 to 11,000 SNPs, 2,500 to 11,000 SNPs, 3,000 to 11,000 SNPs, 3,500 to 11,000 SNPs, 4,000 to 11,000 SNPs, 4,500 to 11,000 SNPs, 5,000 to 11,000 SNPs, 5,550 to 11,000 SNPs, 6,000 to 11,000 SNPs, 6,500 to 11,000 SNPs, 7,000 to 11,000 SNPs, 7,500 to 11,000 SNPs, 8,000 to 11,000 SNPs, 8,500 to 11,000 SNPs, 9,000 to 11,000 SNPs, 9,500 to 11,000 SNPs, or 10,000 to 11,000 SNPs.

In some embodiments, the plurality of SNPs comprises SNPs selected from one or more of the groups consisting of kinship SNPs, ancestry SNPs, identity SNPs, phenotype SNPs, X-SNPs, and Y-SNPs. In some embodiments, the plurality of SNPs comprises kinship SNPs, ancestry SNPs, identity SNPs, phenotype SNPs, X-SNPs, and Y-SNPs. In some embodiments, the plurality of SNPs comprises kinship SNPs. In some embodiments, the plurality of SNPs comprises Y-SNPs. In some embodiments, the plurality of SNPs comprises kinship SNPs and Y-SNPs.

In some of any such embodiments, the plurality of SNPs comprises one or more microhaplotypes. Accordingly, in some embodiments, a microhaplotype is a type of SNP included in the plurality of SNPs. In some embodiments, each microhaplotype comprises one or more SNPs shared on a single amplicon or within proximity of one another on the genome. In general, microhaplotype are biomarkers that are typically less than 300 nucleotides long that display multiple allelic combinations, e.g., multiple SNP-based allelic markers.

In some embodiments, the SNPs do not include SNPs with known medical associations, e.g., associated with known medical conditions, or low minor allele frequencies. By excluding SNPs with known medical associations, e.g., associated with known medical conditions, or low minor allele frequencies, privacy concerns are limited and genetic health data is protected.

In some embodiments, the SNPs comprise SNPs that have been filtered with a plurality of genotype samples. In some embodiments, the SNPs are selected from categories including ancestry SNPs, identity SNPs, kinship SNPs, phenotype SNPs, X-SNPs and Y-SNPs. In some embodiments, the ancestry SNPs include between at or about 10-100 SNPs. In some embodiments, the identity SNPs include between at or about 10-200 SNPs. In some embodiments, the kinship SNPs include between at or about 7,000-12,000 SNPs. In some embodiments, the phenotype SNPs include between at or about 1-50 SNPs. In some embodiments, the X-SNPs include between at or about 10-200 SNPs. In some embodiments, the Y-SNPs include between at or about 10-200 SNPs. In some embodiments, the ancestry SNPs include between at or about 0-10% of the total number of SNPs. In some embodiments, the identity SNPs include between at or about 0-10% of the total number of SNPs. In some embodiments, the kinship SNPs include between at or about 80-100% of the total number of SNPs. In some embodiments, at least or at least about 80%, 81%, 82%, 83%, 84%, 85%, 86%, 87%, 88%, 89%, 90%, 91%, 92%, 93%, 94%, 95%, 96%, 97%, 98%, or 99% of the plurality of SNPs are kinship SNPs. In some embodiments, at least or at least about 85% of the plurality of SNPs are kinship SNPs. In some embodiments, at least or at least about 90% of the plurality of SNPs are kinship SNPs. In some embodiments, at least or at least about 95% of the plurality of SNPs are kinship SNPs. In some embodiments, at least or at least about 99% of the plurality of SNPs are kinship SNPs. In some embodiments, 100% of the plurality of SNPs are kinship SNPs. In some embodiments, the phenotype SNPs include between at or about 0-5% of the total number of SNPs. In some embodiments, the X-SNPs include between at or about 0-5% of the total number of SNPs. In some embodiments, the Y-SNPs include between at or about 0-5% of the total number of SNPs. In some embodiments, the SNPs do not include medically informative or minor allele frequency SNPs. A tag region can be any sequence, such as a universal tag region, a capture tag region, an amplification tag region, a sequencing tag region, a UMI tag region, and the like.

In some embodiments, target sequences are purified and enriched, and a library of the original DNA sample, also referred to as a nucleic acid library, is generated. In some embodiments, the purification combines purification beads with an enzyme to purify the amplified targets from other reaction components. In some embodiments, the purified target sequences are enriched by amplification of the DNA and addition of UDI adapters and sequences required for cluster generation. The UDI adapters can tag DNA with a unique combination of sequences that identify each sample for analysis.

In some embodiments, a nucleic acid library is generated from the amplification products, including the amplification products produced by any of the methods or embodiments described herein. As such, in some embodiments, the nucleic acid library comprises the amplification products generated by amplifying the nucleic acid sample with the plurality of primers that specifically hybridize to a plurality of target sequences collectively comprising a plurality of at least between at or about 5,000 to 50,000 SNPs or at least between at or about 2,000 to 50,000 SNPs.

In some embodiments, nucleic acid libraries or DNA libraries are normalized to quantify and check for quality, and pooled by combining equal volumes of normalized libraries to create a pool of libraries capable of being sequenced together on the same flow cell. In some embodiments, the quantification includes the use of a fluorimetric method. In some embodiments, the quantification includes a quantitative PCR method. After the DNA libraries are pooled, they can be denatured and diluted using a sodium hydroxide (NaOH)-based method, and a sequencing control can be added.

In some embodiments, the nucleic acid libraries are quantitated, normalized, denatured and diluted as per instructions given in Forenseq Kintelligence kit User Guide (Verogen PN: V16000120, the contents of which are hereby incorporated by reference in their entirety).

In some embodiments, the nucleic acid libraries of DNA libraries are prepared for sequencing using massively parallel sequencing using any known suitable method to complement the methods described herein.

In some embodiments, also provided herein is a nucleic acid library constructed using any of the methods described herein.

In some embodiments, the methods provided herein comprise a step of generating a nucleic acid library from the amplification products.

### SEQUENCING AND ANALYSIS

In some aspects, the nucleic acid libraries or DNA libraries described herein can be sequenced using any known suitable method to complement the methods described herein, and are not limited to any particular sequencing platform. In some aspects, the sample disclosed herein can be analyzed using any known suitable method to complement the methods described herein. Exemplary methods of sequencing and methods analysis are described below.

### A. Sequencing

In some embodiments, the methods provided herein comprise a step of sequencing the nucleic acid library generated from the amplification products.

In some embodiments, the technology for sequencing the nucleic acid libraries or DNA libraries created by practicing the methods described herein comprise the use of polymerase-based sequencing by synthesis, ligation based, pyrosequencing or polymerase-based sequencing methods.

In some embodiments, the nucleic acid library is sequenced as per instructions on MiSeq FGx Sequencing System Reference Guide (e.g., document #VD2018006, the contents of which are hereby incorporated by reference in their entirety). In some embodiments, the nucleic acid library that is sequenced as per instructions on MiSeq FGx Sequencing System Reference Guide (e.g., document #VD2018006) is denatured.

In some aspects, the sequencing methods disclosed herein comprise the use of massively parallel sequencing (MPS). In some aspects, the sequencing methods disclosed herein do not comprise the use of whole genome sequencing (WGS). In some aspects, the sequencing methods disclosed herein do not comprise the use of microarrays.

In some embodiments, the sequencing methods disclosed herein detect at or about 90% of the loci of the SNPs.

In some embodiments, the sequencing methods disclosed herein generate an output report comprising the results of the sequencing of the amplification products comprising the plurality of SNPs.

In some embodiments, the sequencing comprises a sequencing plexity of up to 40-plex. In some embodiments, the sequencing comprises a sequencing plexity of 2-plex to 40-plex. In some embodiments, the sequencing comprises a sequencing plexity of 12-plex to 40-plex. In some embodiments, the sequencing comprises a sequencing plexity of 12-plex to 32-plex. In some embodiments, the sequencing comprises a sequencing plexity of 12-plex to 30-plex. In some embodiments, the sequencing comprises a sequencing plexity of 24-plex to 40-plex. In some embodiments, the sequencing comprises a sequencing plexity of 24-plex to 32-plex. In some embodiments, the sequencing comprises a sequencing plexity of 28-plex to 32-plex. In some embodiments, the sequencing comprises a sequencing plexity of 2-plex, 3-plex, 4-plex, 5-plex, 6-plex, 7-plex, 8-plex, 9-plex, 10-plex, 11-plex, 12-plex, 13-plex, 14-plex, 15-plex, 16-plex, 17-plex, 18-plex, 19-plex, 20-plex, 21-plex, 22-plex, 23-plex, 24-plex, 25-plex, 26-plex, 27-plex, 28-plex, 29-plex, 30-plex, 31-plex, or 32-plex. In some embodiments, the sequencing comprises a sequencing plexity of at or about 30-plex. In some embodiments, the sequencing comprises a sequencing plexity of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40-plex. In some embodiments, the sequencing comprises a sequencing plexity of 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45-plex. Sequencing plexity refers to the number of individual samples that are sequenced together, e.g., on a flow cell.

In some embodiments, the sequencing comprises sequencing postmortem samples at a sequencing plexity of between or between about 6-plex and 16-plex. In some embodiments, the sequencing comprises sequencing postmortem samples at a sequencing plexity of between or between about 8-plex and 14-plex. In some embodiments, the sequencing comprises sequencing postmortem samples at a sequencing plexity of between or between about 10-plex and 14-plex. In some embodiments, the sequencing comprises sequencing postmortem samples at a sequencing plexity at or about 10-plex, 11-plex, 12-plex, 13-plex, or 14-plex. In some embodiments, the sequencing comprises sequencing postmortem samples at a sequencing plexity at or about 12-plex.

In some embodiments, the sequencing comprises sequencing antemortem samples at a sequencing plexity of between or between about 24-plex and 40-plex. In some embodiments, the sequencing comprises sequencing postmortem samples at a sequencing plexity of between or between about 26-plex and 38-plex. In some embodiments, the sequencing comprises sequencing postmortem samples at a sequencing plexity of between or between about 28-plex and 36-plex. In some embodiments, the sequencing comprises sequencing postmortem samples at a sequencing plexity at or about 28-plex, 29-plex, 30-plex, 31-plex, 32-plex, 33-plex, or 34-plex. In some embodiments, the sequencing comprises sequencing postmortem samples at a sequencing plexity at or about 32-plex.

### B. Analysis

In some embodiments, the methods provided herein comprise a step of analyzing the sequences of the amplification products.

In some aspects, the methods disclosed herein involve the use of an analysis module that automatically initiates analysis once the sequencing of the samples (i.e. amplification products) is complete. In some embodiments, the analysis module includes Universal Analysis Software (UAS).

In some embodiments, the analysis methods disclosed herein generate an output report comprising the results of the sequencing of the amplification products comprising the plurality of SNPs.

In some embodiments, sequencing results are analyzed using any suitable sequence analysis software available in the art.

In some embodiments, sequencing results are analyzed using the Forenseq Universal Analysis Software, such as version 2.1 or 2.2 or later (Verogen, San Diego, CA) following the instructions outlined in a Forenseq Universal Analysis Software Reference Guide, such as for version 2.2 or later, and provided in, e.g., Reference Guide Document #VD2019002, the contents of which are hereby incorporated by reference in their entirety.

### Genotype and DNA Profile Determination

In some embodiments, the methods provided herein comprise a step of determining the genotypes of the plurality of SNPs, thereby generating a DNA profile.

In some embodiments, a DNA profile is generated by determining the genotypes of the plurality of SNPs.

In some aspects, the output report comprising the results of the sequencing of the amplification products comprising the plurality of SNPs generated by any of the methods described herein can be used to genotype the sample using any known suitable method to complement the methods described herein. In some aspects, the output report comprising the results of the sequencing of the amplification products comprising the plurality of SNPs generated by any of the methods described herein can be used to generate a DNA profile using any known suitable method to complement the methods described herein.

In some embodiments, the DNA profile includes a genotype for each of the plurality of SNPs. In some embodiments, the DNA profile includes a genotype for at least or at least about 80%, 85%, 90%, 91%, 92%, 93%, 94%, 95%, 96%, 97%, 98%, or 99% of the plurality of SNPs. In some embodiments, the DNA profile includes a genotype for at least or at least about 85% of the plurality of SNPs. In some embodiments, the DNA profile includes a genotype for at least or at least about 90% of the plurality of SNPs. In some embodiments, the DNA profile includes a genotype for at least or at least about 95% of the plurality of SNPs. In some embodiments, the DNA profile includes a genotype for at least or at least about 99% or about 100% of the SNPs.

In some embodiments, the methods disclosed herein include determination of hair color, eye color and biogeographical ancestry.

### Degree of Relationship Determination

In some embodiments, the methods provided herein comprise a step of calculating the degree of relationship of the DNA profile to one or more reference DNA profiles, wherein the one or more reference DNA profiles are comprised within a reference set of DNA profiles comprising one or more reference DNA profiles from a relative of the person of interest.

In some aspects, the degree of relationship of the DNA profile described herein can be calculated with reference to one or more reference DNA profiles using any known suitable method to complement the methods described herein.

In some embodiments, the one or more reference DNA profiles are comprised within a reference set of DNA profiles comprising one or more reference DNA profiles from a relative of the person of interest.

In some embodiments, the reference set of DNA profiles comprises up to 5, 10, 15, 20, 25, 30, 35, 40, 45, 50, 55, 60, 65, 70, 75, 80, 85, 90, 95, 100, 125, 150, 175, 200, 300, 400, 500, 600, 700, 800, 900, or 1000 reference DNA profiles. In some embodiments, the reference set of DNA profiles comprises up to 1000 reference DNA profiles. In some embodiments, the reference set of DNA profiles comprises up to 500 reference DNA profiles. In some embodiments, the reference set of DNA profiles comprises up to 250 reference DNA profiles. In some embodiments, the reference set of DNA profiles comprises up to 150 reference DNA profiles. In some embodiments, the reference set of DNA profiles comprises up to 100 reference DNA profiles. In some embodiments, the reference set of DNA profiles comprises up to 75 reference DNA profiles. In some embodiments, the reference set of DNA profiles comprises up to 50 reference DNA profiles. In some embodiments, the reference set of DNA profiles comprises up to 25 reference DNA profiles. In some embodiments, the reference set of DNA profiles comprises up to 15 reference DNA profiles. In some embodiments, the reference set of DNA profiles comprises between 1 and 1,000 reference DNA profiles, between 1 and 500 reference DNA profiles, between 1 and 400 reference DNA profiles, between 1 and 300 reference DNA profiles, between 1 and 250 reference DNA profiles, between 1 and 200 reference DNA profiles, between 1 and 150 reference DNA profiles, between 1 and 100 reference DNA profiles, between 1 and 75 reference DNA profiles, between 1 and 50 reference DNA profiles, between 1 and 25 reference DNA profiles, between 1 and 20 reference DNA profiles, between 1 and 15 reference DNA profiles, between 1 and 10 reference DNA profiles, or between 1 and 5 reference DNA profiles.

In some embodiments, the reference set of DNA profiles comprises at least 1, 2, 3, 4, 5, 6, 7, 8, 9, 10, 15, 20, or 25 reference DNA profiles, and comprises up to 30, 35, 40, 45, 50, 55, 60, 65, 70, 75, 80, 85, 90, 95, 100, 125, 150, 175, 200, 300, 400, 500, 600, 700, 800, 900, or 1000 reference DNA profiles.

In some embodiments, the reference set of DNA profiles comprises 1, 2, 3, 4, 5, 6, 7, 8, 9, 10, 11, 12, 12, 14, 15, 16, 17, 18, 19, or 20 reference DNA profiles.

In some embodiments, the reference set of DNA profiles comprises DNA profiles from a relative of the person of interest. In some embodiments, at least 5%, 10%, 15%, 20%, 25%, 30%, 35%, 40%, 45%, 50%, 55%, 60%, 65%, 70%, 75%, 80%, 85%, 90%, or 95% of the reference DNA profiles in the reference set of DNA profiles is from a relative of the person of interest. In some embodiments, at or about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the reference DNA profiles in the reference set of DNA profiles is from a relative of the person of interest. In some embodiments, 100% of the reference DNA profiles in the reference set of DNA profiles is from a relative of the person of interest. In some embodiments, at least 50% of the reference DNA profiles in the reference set of DNA profiles is from a relative of the person of interest.

In some embodiments, each of the one or more reference DNA profiles from a relative of the person of interest is an antemortem sample. In some embodiments, one or more of the one or more reference DNA profiles from a relative of the person of interest is an antemortem sample. In some embodiments, one or more of the one or more reference DNA profiles from a relative of the person of interest is a postmortem sample. In some embodiments, the one or more reference DNA profiles from a relative of the person of interest comprises a postmortem sample and an antemortem sample.

In some embodiments, each relative of the person of interest in the reference set of DNA profiles is individually a first degree, second degree, third degree, fourth degree, or fifth degree relative of the person of interest. For instance, in an embodiment comprising a reference set of DNA profiles comprising three reference DNA profiles from a relative of the person of interest, each of the three reference DNA profiles can independently be from a first degree relative, a second degree relative, a third degree relative, a fourth degree relative, or a fifth degree relative, e.g., the first reference DNA profile may be from a first degree relative, the second reference DNA profile may be from a third degree relative, and the third reference DNA profile may be from a first degree relative.

In some embodiments, at least 50%, 55%, 60%, 65%, 70%, 75%, 80%, 85%, 90%, or 95% of the one or more reference DNA profiles in the reference set of DNA profiles are related, wherein each of the one or more reference DNA profiles is independently from a first degree relative, a second degree relative, a third degree relative, a fourth degree relative, or a fifth degree relative of each of the other one or more reference DNA profiles in the reference set of DNA profiles.

In some embodiments, at least 5%, 10%, 15%, 20%, 25%, 30%, 35%, 40%, 45%, 50%, 55%, 60%, 65%, 70%, 75%, 80%, 85%, 90%, or 95% of the reference DNA profiles in the reference set of DNA profiles is from a relative of the person of interest, and each of the at least 5%, 10%, 15%, 20%, 25%, 30%, 35%, 40%, 45%, 50%, 55%, 60%, 65%, 70%, 75%, 80%, 85%, 90%, or 95% of the reference DNA profiles in the reference set of DNA profiles is a first degree, second degree, third degree, fourth degree, or fifth degree relative.

In some embodiments, the identity of each relative of the person of interest in the reference set of DNA profiles is known. In some embodiments, the identity of each of the one or more reference DNA profiles in the reference set of DNA profiles is known.

In some embodiments, the reference set of DNA profiles comprises a DNA profile derived from a sample from the person of interest. For instance, in some embodiments, the reference set of DNA profiles comprises a DNA profile derived from a sample from the person of interest prior to disappearance of the person of interest, e.g., in the case of the person of interest being a missing person, or prior to the person of interest becoming a victim of a disaster or conflict. In some embodiments, the DNA profile derived from a sample from the person of interest prior to disappearance of the person of interest, e.g., in the case of the person of interest being a missing person, or prior to the person of interest becoming a victim of a disaster or conflict is used as a positive control for the person of interest since the sample was obtained antemortem or prior to the person of interest's disappearance or victimization and is a sample that is confirmed to be a sample derived from the person of interest. As such, in some embodiments, the reference set of DNA profiles comprises a DNA profile derived from a sample from the person of interest prior to disappearance of the person of interest, e.g., in the case of the person of interest being a missing person, or prior to the person of interest becoming a victim of a disaster or conflict, and, prior to being amplified and/or sequenced, is known to be a sample derived from the person of interest.

In some embodiments, the reference set of DNA profiles is in a database, e.g., a genetic database. In some embodiments, the database is not publicly accessible, i.e., is not accessible by the public. In some embodiments, the database is not a public database, such as a public database that is accessible by law enforcement agencies or third party genealogy services. In some embodiments, the database is not publicly accessible through a subscription service. In some embodiments, the database is not accessible by a third party genealogical service.

In some embodiments, the calculating the degree of relationship of the DNA profile to one or more reference DNA profiles does not comprise accessing a publicly accessible database, e.g., a publicly accessible genetic database. In some embodiments, the calculating the degree of relationship of the DNA profile to one or more reference DNA profiles does not require internet access to access the database comprising the reference set of DNA profiles. In some embodiments, the calculating the degree of relationship of the DNA profile to one or more reference DNA profiles comprises the use of a local database comprising the reference set of DNA profiles. As used herein, “local database” refers to a database stored and accessible only locally, and that is not accessible by the public, e.g., third parties, seeking to query the database.

In some embodiments, the reference set of DNA profiles comprises DNA profiles from two or more unrelated families, e.g., two or more unrelated families (i.e., families that are not related to one another) that each include one or more relatives of a missing person and/or victim of a disaster or conflict. For instance, in the event of a disaster or conflict with multiple victims from multiple families that are unrelated to one another, one or more family members from each of the families may contribute a reference DNA profile within the reference set of DNA profiles. This local reference set of DNA profiles may then be used locally to identify victims of the disaster or conflict from among the multiple unrelated families.

In some embodiments, the reference set of DNA profiles or the database, e.g., the genetic database or the local database, comprises one or more DNA profiles from an individual having an ethnicity of interest. In some embodiments, at least 5%, 10%, 15%, 20%, 25%, 30%, 35%, 40%, 45%, 50%, 55%, 60%, 65%, 70%, 75%, 80%, 85%, 90%, or 95% of the reference DNA profiles in the reference set of DNA profiles is from an ethnicity of interest. In some embodiments, at least 25%, 30%, 35%, 40%, 45%, 50%, 55%, 60%, 65%, 70%, 75%, 80%, 85%, 90%, or 95% of the reference DNA profiles in the reference set of DNA profiles is from an ethnicity of interest. In some embodiments, at least 50%, 55%, 60%, 65%, 70%, 75%, 80%, 85%, 90%, or 95% of the reference DNA profiles in the reference set of DNA profiles is from an ethnicity of interest. In some embodiments, at least 75%, 80%, 85%, 90%, or 95% of the reference DNA profiles in the reference set of DNA profiles is from an ethnicity of interest. In some embodiments, at least 95%, 95%, 97%, 98%, or 99% of the reference DNA profiles in the reference set of DNA profiles is from an ethnicity of interest. In some embodiments, 100% of the reference DNA profiles in the reference set of DNA profiles is from an ethnicity of interest.

In some embodiments, the person of interest has the ethnicity of interest.

In some embodiments, the ethnicity of interest can be any ethnicity, e.g., any ethnicity from anywhere.

In some embodiments, the ethnicity of interest is a rare ethnicity. In some embodiments, the rare ethnicity is represented by at or less than 0.01%, 0.05%, 0.1%, 0.2%, 0.3%, 0.4%, 0.5%, 0.6%, 0.7%, 0.8%, 0.9%, 1%, 2%, 3%, 4%, or 5% of the population in a country of interest or worldwide. In some embodiments, the ethnicity of interest is any ethnicity in a country of interest. In some embodiments, the ethnicity of interest is a dominant ethnicity in a country of interest. In some embodiments, the ethnicity of interest is a minor ethnicity in a country of interest. In some embodiments, the person of interest is from a country of interest.

The country of interest can be any country of interest. In some embodiments, the country of interest is selected from the group consisting of Afghanistan, Albania, Algeria, Andorra, Angola, Antigua and Barbuda, Argentina, Armenia, Australia, Austria, Azerbaijan, Bahamas, Bahrain, Bangladesh, Barbados, Belarus, Belgium, Belize, Benin, Bhutan, Bolivia, Bosnia and Herzegovina, Botswana, Brazil, Brunei, Bulgaria, Burkina Faso, Burundi, Côte d′Ivoire, Cabo Verde, Cambodia, Cameroon, Canada, Central African Republic, Chad, Chile, China, Colombia, Comoros, Congo (Congo-Brazzaville), Costa Rica, Croatia, Cuba, Cyprus, Czechia (Czech Republic), Democratic Republic of the Congo, Denmark, Djibouti, Dominica, Dominican Republic, Ecuador, Egypt, El Salvador, Equatorial Guinea, Eritrea, Estonia, Eswatini (formerly “Swaziland”), Ethiopia, Fiji, Finland, France, Gabon, Gambia, Georgia, Germany, Ghana, Greece, Grenada, Guatemala, Guinea, Guinea-Bissau, Guyana, Haiti, Holy See, Honduras, Hungary, Iceland, India, Indonesia, Iran, Iraq, Ireland, Israel, Italy, Jamaica, Japan, Jordan, Kazakhstan, Kenya, Kiribati, Kuwait, Kyrgyzstan, Laos, Latvia, Lebanon, Lesotho, Liberia, Libya, Liechtenstein, Lithuania, Luxembourg, Madagascar, Malawi, Malaysia, Maldives, Mali, Malta, Marshall Islands, Mauritania, Mauritius, Mexico, Micronesia, Moldova, Monaco, Mongolia, Montenegro, Morocco, Mozambique, Myanmar (formerly Burma), Namibia, Nauru, Nepal, Netherlands, New Zealand, Nicaragua, Niger, Nigeria, North Korea, North Macedonia, Norway, Oman, Pakistan, Palau, Palestine State, Panama, Papua New Guinea, Paraguay, Peru, Philippines, Poland, Portugal, Qatar, Romania, Russia, Rwanda, Saint Kitts and Nevis, Saint Lucia, Saint Vincent and the Grenadines, Samoa, San Marino, Sao Tome and Principe, Saudi Arabia, Senegal, Serbia, Seychelles, Sierra Leone, Singapore, Slovakia, Slovenia, Solomon Islands, Somalia, South Africa, South Korea, South Sudan, Spain, Sri Lanka, Sudan, Suriname, Sweden, Switzerland, Syria, Tajikistan, Tanzania, Thailand, Timor-Leste, Togo, Tonga, Trinidad and Tobago, Tunisia, Turkey, Turkmenistan, Tuvalu, Uganda, Ukraine, United Arab Emirates, United Kingdom, United States of America, Uruguay, Uzbekistan, Vanuatu, Venezuela, Vietnam, Yemen, Zambia, and Zimbabwe. In some embodiments, the country of interest is the United States of America.

In some embodiments, the DNA-based kinship analysis described herein includes the use of a local database. In some embodiments, the DNA-based kinship analysis described herein allows for generation of a report with minimal user input. In some embodiments, the DNA-based kinship analysis described herein comprises the use of an algorithm to calculate kinship coefficient. In some embodiments, the kinship coefficient determines the relationship status of the sample or DNA profile to a reference DNA profile on a database. For instance, in some embodiments, the kinship coefficient indicates whether each of one or more identified genetic relatives is likely to be a great great grandmother, a great great grandfather, a great grandfather, a great grandmother, a grandmother, a grandfather, a first cousin, a first cousin once removed, or a second cousin, based on the relative value of the kinship coefficient. In some embodiments, the reference DNA profiles are part of a genealogy database.

In some embodiments, the DNA-based kinship analysis described herein comprises identifying genetic relatives to at or about the 1^{st}, 2^{nd}, 3^{rd}, 4^{th}, or 5^{th} degree. In some embodiments, the DNA-based kinship analysis described herein comprises identifying genetic relatives to more than the 1^{st}, 2^{nd}, 3^{rd}, 4^{th}, or 5^{th} degree. In some embodiments, the DNA-based kinship analysis described herein comprises identifying the degree of relationship between the person of interest and one or more of the one or more reference DNA profiles in the reference set of DNA profiles. For instance, in some embodiments, the method comprises identifying that the person of interest is independently a first degree relative, a second degree relative, a third degree relative, a fourth degree relative, or a fifth degree relative of one or more of the one or more reference DNA profiles. A first degree relative of a person is the person's parent (e.g., father or mother), full sibling (e.g., sister or brother), or child (e.g., son or daughter). A second degree relative of a person is someone who shares approximately 25% of the person's genes, such as the person's grandparents, aunt, uncle, niece, nephew, grandchildren, or a half sibling. A third degree relative of a person is someone who shares approximately 12.5% of the person's genes, such as great-grandparents, first cousins, and great-grandchildren. A fourth degree relative includes, e.g., a first cousin once removed, a half great uncle, a half great aunt, a half great niece, a half great nephew, and a half first cousin. A fifth degree relative includes, e.g., a second cousin, a half first cousin once removed, and a first cousin twice removed.

In some embodiments, the DNA-based kinship analysis described herein comprises generating a family tree comprising the DNA profile in relation to one or more DNA profiles. The family tree can be generated using any available means or methodologies.

In some embodiments, the DNA-based kinship analysis described herein comprises identifying suspects through common ancestors.

In some embodiments, the calculating the degree of relationship comprises calculating the degree of relationship between the DNA profile, i.e., the DNA profile from the person of interest, and one or more reference DNA profiles that are comprised within a reference set of DNA profiles, e.g., a reference set of DNA profiles comprising one or more reference DNA profiles from a relative of the person of interest.

In some embodiments, the calculating the degree of relationship comprises calculating the degree of relationship between the DNA profile, i.e., the DNA profile from the person of interest, and one or more reference DNA profiles that are comprised within a reference set of DNA profiles, e.g., a reference set of DNA profiles comprising one or more reference DNA profiles from a relative of the person of interest, by comparing a set of SNPs that is or comprises one or more Y-SNPs. In some embodiments, the one or more Y-SNPs comprises at or at least 5, 10, 15, 20, 25, 30, 35, 40, 45, 50, 55, 60, 65, 70, 75, 80, 81, 82, 83, 84, or 85 Y-SNPs. In some embodiments, the one or more Y-SNPs is or comprises 85 Y-SNPs. By comparing a set of SNPs that comprises Y-SNPs between the DNA profile from a biological male and one or more reference DNA profiles from biological males, a likelihood ratio for the male lineage can be determined.

Likeihood ratios (LRs) and kinship values can be calculated using any approach or algorithm(s) known in the art. In some embodiments, the likelihood ratio is calculated using the algorithms pedprobr (Brustad et al., Int. J. Legal Med., 2021, 135:117-129, the content of which is hereby incorporated by reference in its entirety) and dvir (Vigeland et al., Scientific Reports, 2021, 11:13661, the content of which is hereby incorporated by reference in its entirety). In some embodiments, an average of the population frequencies from the Genome Aggregation Database (gnoMAD) (Karczewski et al., Nature, 2020, 581:434-443, the content of which is hereby incorporated by reference in its entirety) v3.0 is used in the LR calculations. In some embodiments, no mutation model is used, and theta is set to 0 when the SNPs chosen for the analysis have low linkage disequilibrium (Karczewski et al., supra, the content of which is hereby incorporated by reference in its entirety). In some embodiments, the LR is calculated as follows:

$$LR = \frac{P ( D \vert {H}_{r} )}{P ( D \vert {H}_{u} )}$$

where D represents the genotypes, Hᵣ represents the hypothesis that the individuals are related, and Hᵤ represents the hypothesis that the individuals are unrelated. The related hypothesis is signified by a pedigree where the unidentified individual is tested as the relative. The unrelated hypothesis is signified by a Hardy-Weinberg equilibrium calculation. In some embodiments, an LR value is calculated per locus and then multiplied across loci, which results in a final LR for the relationship. To improve computational efficiency, each locus LR can be converted to logarithm and loci LRs are summed. However, due to the high plexity of this platform and the, often, high degradation level and/or low input of PM samples in mass fatality incident (MFI) cases, stochastic effects during PCR cause allele drop out and can introduce a situation where the allele combination between two individuals at a locus appear impossible. This typically occurs in parent-offspring relationships where each individual in the relationship is genotyped as homozygous for different alleles but one or both are actually heterozygous (e.g., parent has genotype AA and child as genotype BB when the parent or both parent and child are actually AB). Genotyping error and de novo mutation are also possible causes of such a case. This situation results in a LR of 0 and can result in an overall LR of 0 given that the final LR is the product of the loci LRs. To ensure locus LRs are not 0, in some embodiments a modification is made to pedprobr. Accordingly, in some embodiments, the likelihood ratio for the locus in these cases is calculated as follows:

$$LR = \frac{0.001}{{p}^{2} {q}^{2}}$$

where 0.001 represents a genotyping error rate, p is the allele frequency of the allele 1, and q is the allele frequency of allele 2 (Galván-Femenía et al., Heredity, 2021, 126:537-547, the content of which is hereby incorporated by reference in its entirety). Allele 1 and allele 2, also referred to as a first allele and a second allele or a locus, can be any two alleles of interest for a particular locus.

In some embodiments, the LR is calculated as described in Galván-Femenía et al., Heredity, 2021, 126:537-547, the content of which is hereby incorporated by reference in its entirety.

In some embodiments, the calculating the degree of relationship comprises calculating a likelihood ratio for sharing a Y chromosome between the DNA profile, i.e., the DNA profile from the person of interest, and one or more reference DNA profiles that are comprised within a reference set of DNA profiles, e.g., a reference set of DNA profiles comprising one or more reference DNA profiles from a relative of the person of interest. In some embodiments, the calculating the likelihood ratio for sharing a Y chromosome comprises comparing a set of SNPs that is or comprises one or more Y-SNPs. In some embodiments, the one or more Y-SNPs comprises at or at least 5, 10, 15, 20, 25, 30, 35, 40, 45, 50, 55, 60, 65, 70, 75, 80, 81, 82, 83, 84, or 85 Y-SNPs. In some embodiments, the one or more Y-SNPs is or comprises 85 Y-SNPs.

In some embodiments, the calculating the likelihood ratio for sharing a Y chromosome comprises calculating a kinship coefficient based on one or more Y-SNPs from within the plurality of SNPs. In some embodiments, the calculating the likelihood of sharing a Y chromosome comprises calculating a log likelihood by providing the DNA profile and one or more reference DNA profiles that are comprised within a reference set of DNA profiles as input, e.g., as input to PC-Relate. In some embodiments, the calculating the likelihood of sharing a Y chromosome can allow for identifying matching Y chromosomes that are shared between the DNA profile, e.g., the DNA profile of the person of interest, and one or more of the one or more reference DNA profiles, which can then be used to determine the likelihood ratios for the male lineage of the person of interest.

In some embodiments, this includes the use of kinship coefficients that calculate the measurement of relationship between two samples, e.g., between the DNA profile and one of the one or more reference DNA profiles. In some embodiments, the methods provided herein involve calculating the kinship coefficient using a kinship model built from, e.g., a public genealogy database, for instance using PC-AiR or a modified PC-AiR method, and determining kinship on a local set of target samples, e.g., using PC-Relate, rather than on a public database using an expansive set of publicly accessible samples. This also includes, in some embodiments, calculating likelihood ratios (LRs) for each comparison. A likelihood ratio (LR) is a standard measure of relatedness in the field of forensics, for instance.

In some embodiments, a whole genome kinship coefficient, shared cMs, and longest segment cMs are calculated using PC-Relate (Conomos et al., American Journal of Human Genetics, 2016, 98:127-148, the content of which is hereby incorporated by reference in its entirety) and PC-AiR (Conomos et al., Genetic Epidemiology, 2015, 39:276-293, the content of which is hereby incorporated by reference in its entirety), such as described previously in Snedecor et al., Forensic Sci. Int. Genet., 2022, 61:102769, the content of which is hereby incorporated by reference in its entirety. This method is useful when the relationship between two individuals is unknown, thereby not requiring a pedigree, which is applicable to situations such as with missing persons or victims of conflict.

In some embodiments, the PC-AiR method first takes a set of genotyped individuals and separates them into two nonoverlapping subsets: one set containing unrelated individuals that represent ancestries of all individuals (unrelated subset), the other set containing individuals that have at least one relative within the first subset (related subset). To build the unrelated subset, a modification was made to the original PC-AiR method to improve computational efficiency in building the model. Samples with none or the fewest relatives are added to the unrelated subset, while those with more relatives are excluded from the unrelated subset. This is performed by calculating a kinship value as in Conomos et al., Genetic Epidemiology, 2015, 39:276-293, the content of which is hereby incorporated by reference in its entirety, per pair and categorizing each individual as having a relative or not based on stringent thresholds: a relative is considered if the kinship value is greater than 0.01 and not related if the kinship value is less than-0.025. Samples with less than 5% missing SNP data are excluded. Next, principal component analysis (PCA) is performed on the unrelated subset, then values are predicted along components of variations for all individuals in the related subset based on genetic similarities with individuals in the unrelated subset. The resulting components represented a model that can be used in place of static population frequencies to identify matches in a set of unknown individuals.

In some embodiments, the PC-Relate method uses the principal components from PC-AiR and separates genetic correlations into two components: one for the sharing of alleles that are identical by descent from recent common ancestors and another for allele sharing due to more distant common ancestors. The components from PC-AiR are used to estimate allele frequencies based on the individual's ancestral background using linear regression instead of static population frequencies, such as those from gnoMAD. For two individuals, I and j, a kinship coefficient, [symbol] , is then calculated using the estimated allele frequencies, û, from the PC-AiR model as follows

$$\text{?} = \frac{{\sum }_{s ∈ S} ( {g}_{is} - 2 {\hat{u}}_{is} ) ( {g}_{js} - 2 {\hat{u}}_{js} )}{4 {{\sum }_{s ∈ S} [ {\hat{u}}_{is} ( 1 - {\hat{u}}_{is} ) \cdot {\hat{u}}_{js} ( 1 - {\hat{u}}_{js} ) ]}^{1 / 2}}$$

where s is a SNP in S SNPs that were typed in both individuals, gᵢₛ and gⱼₛ are the number of reference alleles in i and j at SNP s, respectively, and ûᵢₛ and ûⱼₛ are the expected allele frequencies calculated by PC-AiR for i and j at SNP s, respectively. This algorithm is termed “whole genome kinship,” as it considers the entire genome as one segment of relatedness, signified by a whole genome kinship coefficient. This whole genome kinship coefficient is used to identify relationships when referring to the whole genome kinship algorithm. When the number of SNPs shared between two individuals is less than 6000, a whole genome kinship coefficient of more than 0.031 between two individuals is required to be considered relatives in this study.

Accordingly, in some embodiments, the calculating the degree of relationship comprises calculating a kinship coefficient using a whole genome kinship alogirthm as follows:

$$\text{?} = \frac{{\sum }_{s ∈ S} ( {g}_{is} - 2 {\hat{u}}_{is} ) ( {g}_{js} - 2 {\hat{u}}_{js} )}{4 {{\sum }_{s ∈ S} [ {\hat{u}}_{is} ( 1 - {\hat{u}}_{is} ) \cdot {\hat{u}}_{js} ( 1 - {\hat{u}}_{js} ) ]}^{1 / 2}}$$

where the person of interest and a reference DNA profile are i and j, [symbol] is the kinship coefficient, û is the estimated allele frequencies, s is a SNP in S SNPs that were typed in both individuals, gᵢₛ and gⱼₛ are the number of reference alleles in i and j at SNP s, respectively, and ûᵢₛ and ûⱼₛ are the expected allele frequencies calculated by PC-AiR for i and j at SNP s, respectively.

In some embodiments, the calculating the degree of relationship comprises calculating a kinship coefficient using a “windowed kinship” approach. See Snedecor et al., Forensic Sci Int Genet 2022, 61, 102769, doi: 10.1016/j.fsigen.2022.102769, the content of which is hereby incorporated by reference in its entirety. Windowed kinship involves calculating windows of kinship across the genome to find shared kinship segments. This is performed by enumerating all possible windows within each chromosome and calculating a kinship coefficient for all windows. These windows are then filtered by a minimum kinship coefficient threshold and included in the shared cMs calculation. The filtered segments are then iterated and stretches of SNPs sharing at least one allele and two alleles are categorized separately. Total shared cMs is then calculated across all segments. Total shared cM and the longest segment of cM are used to identify relationships when referring to the windowed kinship algorithm. When the number of SNPs shared between two individuals is between 6000 and 8000, the shared cM value must be above 180 and the longest segment of cM must be above 30 to be considered a relationship. When the number of SNPs shared between two individuals is between 8000 and 9000, the shared cM value must be above 150 and the longest segment of cM must be above 30 to be considered a relationship. When the number of SNPs shared between two individuals is 9000 or more, the shared cM value must be above 140 and the longest segment of cM must be above 30 to be considered a relationship. The whole genome kinship coefficient can be used to filter at any number of SNPs shared. However, Snedecor et al., supra, observed a higher specificity when filtering on shared cM and longest segment cM (e.g., using windowed kinship) when the SNP overlap was greater than 6000, particularly for higher degrees of relationships.

More simply, the number of SNPs typed between two individuals (SNP overlap) can be used to decide when to use the whole genome kinship algorithm (<6000 SNPs overlap) and when to use the windowed kinship algorithm (>6000 SNPs overlap). And, when one algorithm is decided upon based on that SNP overlap, a value or a set of values are used to filter the data to identify relationships, depending on which algorithm was chosen. The cutoffs for both whole genome kinship and windowed kinship are chosen to ensure a high sensitivity but more importantly, a high specificity as demonstrated in Snedecor et al., supra. Lowering these thresholds may capture more relationships (i.e., increase sensitivity) but is expected to introduce more false positive hits, particularly for more distant relationships (e.g., fourth-and fifth-degree).

In some embodiments, the calculating the degree of relationship comprises calculating a kinship coefficient for the DNA profile, e.g., the DNA profile from the person of interest, and one of the one or more reference DNA profiles. In some embodiments, the degree of relationship, e.g., kinship coefficient, is calculated for the DNA profile and each of the one or more reference DNA profiles. In some embodiments, a likelihood ratio is calculated by dividing the probability of the query, e.g., the DNA profile of the person of interest, and the target, e.g., one of the one or more reference DNA profiles, being related, by the probability of the query and the target being unrelated based on the observed genotypes in the two samples. The results can then be filtered based on the kinship coefficient and the LR to identify the most probable relationship(s) and to eliminate false matches from among the one or more reference DNA profiles that are comprised within a reference set of DNA profiles.

In some embodiments, the calculating the likelihood ratio comprises comparing the plurality of SNPs between the DNA profile and the one or more reference DNA profiles. In some embodiments, the calculating the degree of relationship comprises calculating a kinship coefficient based on kinship SNPs from within the plurality of SNPs. In some embodiments, the calculating the degree of relationship comprises calculating a kinship coefficient based on kinship SNPs from within the plurality of SNPs and a kinship coefficient based on the Y-SNPs from within the plurality of SNPs. In some embodiments, the calculating the degree of relationship comprises calculating a kinship coefficient based on the Y-SNPs from within the plurality of SNPs. In some embodiments, the calculating the likelihood ratio comprises comparing a set of SNPs comprising kinship SNPs from within the plurality of SNPs between the DNA profile and the one or more reference DNA profiles.

In some embodiments, the person of interest is biologically male and the method further comprises calculating a likelihood ratio for sharing a Y chromosome between the DNA profile and the one or more reference DNA profiles. In some embodiments, the calculating a likelihood ratio for sharing a Y chromosome comprises comparing a set of SNPs that comprises one or more Y-SNPs between the DNA profile and the one or more reference DNA profiles. In some embodiments, the one or more Y-SNPs are comprised within the plurality of SNPs. In some embodiments, the one or more Y-SNPs comprises at least 25, 50, 75, or 100 Y-SNPs. In some embodiments, the one or more Y-SNPs comprises at least 20, 25, 30, 35, 40, 45, 50, 55, 60, 65, 70, 75, 80, 81, 82, 83, 84, or 85 Y-SNPs. In some embodiments, the one or more Y-SNPs comprises 85 Y-SNPs. In some embodiments, calculating the likelihood ratio for sharing a Y chromosome comprises dividing the probability of the DNA profile and a reference DNA profile from among the one or more reference DNA profiles sharing a Y chromosome by the probability of the DNA profile and the reference DNA profile not sharing a Y chromosome based on the genotypes of the one or more Y-SNPs.

In some embodiments, the calculating the degree of relationship, e.g., kinship coefficient, comprises the use of a principal component analysis (PCA) method. In some embodiments, the degree of relationship is calculated using a kinship model. In some embodiments, the degree of relationship is calculated using a kinship model that is trained using a PCA method. In some embodiments, the PCA method for training the kinship model is PCA. In some embodiments, the PCA method for training the kinship model involves PCA. In some embodiments, the PCA method for training the kinship model is one that can account for sample relatedness, for instance known or cryptic relatedness that can arise from family structure across samples. In some embodiments, the PCA method is PC-AiR, which can allow for ancestry determination in the presence of known or cryptic relatedness. See, e.g., Conomos et al., Robust Inference of Population Structure for Ancestry Prediction and Correction of Stratification in the Presence of Relatedness, Genet Epidemiol., 2015, 39 (4): 276-293, the contents of which are hereby incorporated by reference. In some embodiments, the PCA method is a modified PC-AiR method, such as described herein.

In some embodiments, the kinship model is built using a training database. In some embodiments, the training database is a genetic database. In some embodiments, the training database is a genealogy database. In some embodiments, the training database is a publicly accessible database. In some embodiments, the training database comprises between 1 and 10 million or more training DNA profiles. In some embodiments, the training database comprises at or about or at least or at least about 1, 5, 25, 50, 75, 100, 500, 1,000, 1,500, 2,000, 3,000, 4,000, 5,000, 10,000, 20,000, 30,000, 40,000, 50,000, 75,000, 100,000, 125,000, 150,000, 175,000, 200,000, 225,000, 250,000, 275,000, 300,000, 400,000, 500,000, 600,000, 700,000, 800,000, 900,000, 1,000,000, 1,250,000, 1,500,000, 1,750,000, 2,000,000, 3,000,000, 4,000,000, 5,000,000, or 10,000,000 training DNA profiles, or a range between any two of the preceding values. In some embodiments, the training database comprises up to or up to about 100, 500, 1,000, 1,500, 2,000, 3,000, 4,000, 5,000, 10,000, 20,000, 30,000, 40,000, 50,000, 75,000, 100,000, 125,000, 150,000, 175,000, 200,000, 225,000, 250,000, 275,000, 300,000, 400,000, 500,000, 600,000, 700,000, 800,000, 900,000, 1,000,000, 1,250,000, 1,500,000, 1,750,000, 2,000,000, 3,000,000, 4,000,000, 5,000,000, or 10,000,000 training DNA profiles. In some embodiments, the training database comprises between 5,000 and 500,000, or between 10,000 and 500,000, or between 15,000 and 500,000, or between 20,000 and 500,000, or between 25,000 and 500,000, or between 25,000 and 400,000, or between 25,000 and 300,000, or between 25,000 and 250,000, or between 50,000 and 500,000, or between 50,000 and 400,000, or between 50,000 and 300,000, or between 50,000 and 250,000 training DNA profiles.

In some embodiments, the PCA method is PC-AiR, and the training database comprises at least 1 and up to 100, 200, 300, 400, 500, 600, 700, 800, 900, 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,500, 4,000, 4,500, or 5,000 training DNA profiles, or a range between any two of the preceding values.

In some embodiments, the PCA method is the modified PC-Air method, and the training database comprises at or about or at least or at least about 3,000, 4,000, 5,000, 10,000, 20,000, 30,000, 40,000, 50,000, 75,000, 100,000, 125,000, 150,000, 175,000, 200,000, 225,000, 250,000, 275,000, 300,000, 400,000, 500,000, 600,000, 700,000, 800,000, 900,000, 1,000,000, 1,250,000, 1,500,000, 1,750,000, 2,000,000, 3,000,000, 4,000,000, 5,000,000, or 10,000,000 training DNA profiles, or a range between any two of the preceding values.

In some embodiments, accessing the training database does not require internet access. In some embodiments, training the kinship model does not require internet access. In some embodiments, the training database is accessible locally.

In some embodiments, the kinship model is trained by applying the PCA method to the training database. In some embodiments, the training DNA profiles include genotypes of the plurality of SNPs. In some embodiments, the kinship model includes principal components (PCs) obtained for the training database using the PCA method.

In some aspects, given a training database of training DNA profiles, PC-AiR and the modified PC-AiR method can both identify a sufficiently acceptable unrelated sample set of training DNA profiles from the training database that is as close to as large as possible while also sampling well from all ancestral backgrounds present in the training database. In some embodiments, PC-AiR and the modified PC-AiR method can both identify a set of unrelated samples, e.g., training DNA profiles, within the training database. In some embodiments, the set of unrelated samples is one that samples all or nearly all ancestral backgrounds present in the training database.

In some embodiments, PC-AiR and the modified PC-AiR method both include an initial step of estimating kinship between all pairs of samples in the training database. In some embodiments, kinship coefficients are estimated. In some embodiments, kinship coefficients are estimated using a simplified kinship estimation method called “KING-Robust”.

In some embodiments, PC-AiR then proceeds into subsequent steps that include: (1) initializing a set “U” with all of the samples from the training database; (2) scanning the set to calculate, for each sample, how many samples that sample is related to in U (referred to as “R”), and how many samples it is “ancestrally diverged” from in U (referred to as “D”); (3) selecting the sample with the highest R and, if there are multiple samples having the highest R, then selecting the sample having the highest R and the lowest D; (4) removing the selected sample from U; and (5) repeating from step (2). For instance, using PC-AiR, if there are 50,000 samples, the process will look at 50,000² data points in the first iteration, 49,999² data points in the second iteration, and so on until there are no more related samples in the set, which may proceed down to, e.g., 20,000² or 10,000² data points. In some embodiments, this procedure continues until U contains only unrelated samples.

In some embodiments, PC-AiR considers samples as related based on estimated kinship. In some embodiments, samples with estimated kinship coefficient ≥0.025 are considered related.

In some embodiments, PC-AiR considers samples as ancestry-diverged based on estimated kinship. In some embodiments, samples with estimated kinship coefficient <0.025 are considered ancestry-diverged.

In some embodiments, PC-AiR comprises the steps of: (1) estimating kinship coefficients between all pairs of samples, e.g., training DNA profiles, of the training database, wherein pairings with a kinship coefficient >0.025 are identified as related and pairings with a kinship coefficient <−0.025 are identified as ancestry-diverged; (2) initializing an unrelated sample set that includes all samples; and (3) iteratively: (i) identifying the set in the unrelated sample set that have the most related samples in the unrelated sample set, thereby designated as X, (ii) identifying the set of samples in X that have the least ancestry-diverged pairings compared to samples in the unrelated sample set, thereby designated as Y; and (iii) if Y has zero samples, then terminating the process, or, if Y has at least one sample, then randomly selecting one sample from Y to remove from U, and repeat beginning at step (3) (i).

In some embodiments, the modified PC-AiR method comprises one or more adjustments compared to PC-AiR. In some embodiments, whether samples are related is defined more stringently in the modified PC-AiR method. In some embodiments, the modified PC-AiR method considers samples as related if estimated kinship coefficient is ≥0.01.

In some embodiments, the modified PC-AiR method considers samples as ancestry-diverged based on the estimated kinship coefficients. In some embodiments, samples with estimated kinship coefficient <0.025 are considered ancestry-diverged.

In some embodiments, the modified PC-AiR method comprises removing all samples with >5% missing genotypes (e.g., more than 5% of the SNPs in the DNA profile) in order to make sure that each sample is sufficiently informative.

In some embodiments, the modified PC-AiR method comprises steps of (1) for each sample, computing: “R” which is the total number of related samples in the training database, “D” which is the number of ancestral diverged samples in the database, and “S” which is the set of related samples; (2) ranking all samples by R (ascending) and D (descending); (3) iterating through the ranked list of samples and: (i) if the sample is not in the “related” set, adding it to the unrelated set and adding all samples from S (i.e., DNA profiles related to the sample) to the related set; or (ii) if the sample is in the “related” set, disregarding the sample and moving to the next sample. In some aspects, this modified PC-AiR method allows for a process that is largely linear complexity (i.e., the runtime expands linearly with the number of samples) rather than exponential

In some embodiments, the modified PC-AiR method comprises steps of: (1) estimating kinship coefficients between all pairs of samples, e.g., DNA profiles, of the training database, wherein pairings with a kinship coefficient >0.01 are identified as related and pairings with a kinship coefficient <−0.025 are identified as ancestry-diverged; (2) removing all DNA profiles that have ≥5% missing data; and (3) ranking all DNA profiles by identifying each DNA profile with a ranking value. In some embodiments, the ranking value is determined based on the number of related DNA profiles in the full database that is ranked from least to most, and ties are broken by the number of ancestry-diverged DNA profiles in the full database as ranked from most to least. In some embodiments, step (3) involves going iteratively through the ranked DNA profiles, and for each DNA profile: (i) if the DNA profile is not yet in the related sample set, adding it to the unrelated sample set and adding all related DNA profiles to the related sample set, and (ii) if the DNA profile is already in the related sample set, then skipping to the next DNA profile, and repeating beginning at step (3) (i).

In some embodiments, following determination of the unrelated sample set using either PC-AiR or the modified PC-AiR method, PCA is applied to the unrelated sample set in order to train the kinship model. In some embodiments, the kinship model further includes PC values that are calculated for the related sample set. In some embodiments, the PC values for the related sample set are determined based on the PCs obtained for the unrelated sample set.

In some embodiments, PCA is applied to the entire training database for building the kinship model.

In some embodiments, the provided methods involve training the kinship model.

In some embodiments, the provided methods do not involve training the kinship model. In some embodiments, the kinship model is trained prior to the calculating the degree of relationship, e.g., kinship coefficient.

In some embodiments, accessing the kinship model does not require internet access. In some embodiments, the kinship model is accessible locally.

In some embodiments, the degree of relationship, e.g., kinship coefficient, is calculated using the kinship model. In some embodiments, the degree of relationship is calculated using the PCs of the kinship model. In some embodiments, calculating the degree of relationship involves obtaining PC values for the DNA profile, e.g., the DNA profile of the person of interest. In some embodiments, calculating the degree of relationship involves obtaining PC values for the reference DNA profile or profiles. In some embodiments, the degree of relationship is calculated using the PC values for the DNA profile. In some embodiments, the degree of relationship is calculated using the PC values for the DNA profile and the reference DNA profile or profiles.

In some embodiments, the degree of relationship, e.g., kinship coefficient, is calculated using PC-Relate. See, e.g., Conomos et al., Model-free Estimation of Recent Genetic Relatedness, Am. J. Hum. Genet., 98 (1): 127-148 (2016), the content of which is hereby incorporated by reference in its entirety. In some embodiments, the degree of relationship is calculated by providing the DNA profile, e.g., the DNA profile of the person of interest, as input to PC-Relate. In some embodiments, the degree of relationship is calculated by providing the kinship model, e.g., the PCs, and the DNA profile as input to PC-Relate. In some embodiments, the reference DNA profile or profiles are further provided as input to PC-Relate.

In some embodiments, the degree of relationship, e.g., kinship coefficient, is calculated locally. In some embodiments, calculating the degree of relationship does not require internet access.

In some embodiments, the methods described herein further comprises identifying the person of interest. In some embodiments, identifying the person of interest comprises identifying the person of interest by the person of interest's legal name. In some embodiments, identifying the person of interest comprises identifying the person of interest by the person of interest's familial relationship to one or more known persons in the reference set of DNA profiles. For instance, in some embodiments, identifying the person of interest comprises identifying that the person of interest is the son or daughter of a specific known person, and/or is the full sibling of a specific known person.

### Kits

Provided herein are kits comprising any of the primers, reagents or compositions described herein, which may further comprise instruction(s) on methods of using the kit, such as uses described herein. The kits described herein may also include other materials desirable from a commercial and user standpoint, including other buffers, diluents, filters, and package inserts with instructions for performing any methods described herein.

In some embodiments, provided herein is a kit comprising at least one container means, wherein the at least one container means comprises any of the plurality of primers as described herein.

### Exemplary Embodiments

Among the exemplary embodiments provided herein are:

1. A method for performing DNA-based kinship analysis, comprising:
providing a nucleic acid sample from a person of interest,
amplifying the nucleic acid sample with a plurality of primers that specifically hybridize to a plurality of target sequences collectively comprising a plurality of at least between at or about 2,000 to 50,000 single nucleotide polymorphisms (SNPs), thereby generating amplification products, wherein the amplification is carried out in one or more multiplex PCR reactions,
generating a nucleic acid library from the amplification products,
sequencing the nucleic acid library generated from the amplification products,
analyzing the sequences of the amplification products,
determining the genotypes of the plurality of SNPs, thereby generating a DNA profile, and
calculating the degree of relationship of the DNA profile to one or more reference DNA profiles, wherein the one or more reference DNA profiles are comprised within a reference set of DNA profiles comprising one or more reference DNA profiles from a relative of the person of interest.

2. A method for performing DNA-based kinship analysis, comprising:
providing a nucleic acid sample from a person of interest,
amplifying the nucleic acid sample with a plurality of primers that specifically hybridize to a plurality of target sequences collectively comprising a plurality of at least between at or about 2,000 to 50,000 single nucleotide polymorphisms (SNPs), thereby generating amplification products, wherein the amplification is carried out in one or more multiplex PCR reactions,
generating a nucleic acid library from the amplification products,
sequencing the nucleic acid library generated from the amplification products,
determining the genotypes of the plurality of SNPs, thereby generating a DNA profile, and
calculating the degree of relationship of the DNA profile to one or more reference DNA profiles, wherein the one or more reference DNA profiles are comprised within a reference set of DNA profiles comprising one or more reference DNA profiles from a relative of the person of interest.

3. The method of embodiment 1 or embodiment 2, wherein the sequencing is conducted using massively parallel sequencing (MPS).

4. The method of any one of embodiments 1-3, wherein the sequencing does not comprise whole genome sequencing (WGS).

5. The method of any one of embodiments 1-4, further comprising generating a family tree comprising the DNA profile in relation to one or more DNA profiles.

6. A method of constructing a nucleic acid library for a person of interest, comprising:
providing a nucleic acid sample from a person of interest,
amplifying the nucleic acid sample with a plurality of primers that specifically hybridize to a plurality of target sequences collectively comprising a plurality of at least between at or about 2,000 to 50,000 single nucleotide polymorphisms (SNPs), thereby generating a nucleic acid library comprising amplification products, wherein the amplification is carried out in one or more multiplex PCR reactions.

7. The method of embodiment 6, further comprising a step of sequencing the amplification products to produce a DNA profile for the person of interest.

8. A method of constructing a nucleic acid library for a reference DNA sample, comprising:
providing a nucleic acid sample from a relative of a person of interest,
amplifying the nucleic acid sample with a plurality of primers that specifically hybridize to a plurality of target sequences collectively comprising a plurality of at least between at or about 2,000 to 50,000 single nucleotide polymorphisms (SNPs), thereby generating a nucleic acid library comprising amplification products, wherein the amplification is carried out in one or more multiplex PCR reactions.

9. The method of embodiment 8, wherein the relative is a first-, second-, third-, fourth-, or fifth-degree relative of the person of interest.

10. The method of embodiment 8 or embodiment 9, wherein the relative is a first-, second-, or third-degree relative of the person of interest.

11. The method of any one of embodiments 1-10, wherein the nucleic acid sample comprises genomic DNA.

12. The method of any one of embodiments 1-11, wherein the nucleic acid sample comprises one or more enzyme inhibitors.

13. The method of embodiment 12, wherein the one or more enzyme inhibitors comprise one or more inhibitors selected from the group consisting of hematin, heme, humic acid, indigo, tannic acid, collagen, calcium, and hydroxyapatite.

14. The method of any one of embodiments 1-13, wherein the nucleic acid sample comprises low-quality nucleic acid molecules and/or low quantity nucleic acid molecules.

15. The method of embodiment 14, wherein the low quality nucleic acid molecules are degraded genomic DNA and/or fragmented genomic DNA.

16. The method of embodiment 14 or embodiment 15, wherein the low quality nucleic acid molecules have a degradation index (DI) of at or at least 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200.

17. The method of embodiment 14 or embodiment 15, wherein the low quality nucleic acid molecules have a DI of at least 1 and up to or less than 158.3.

18. The method of any one of embodiments 1-13, wherein the nucleic acid sample comprises high quality nucleic acid molecules.

19. The method of embodiment 18, wherein the high quality nucleic acid molecules have a DI of less than 1.

20. The method of any one of embodiments 1-19, wherein the person of interest is a missing person.

21. The method of any one of embodiments 1-19, wherein the person of interest a victim of a disaster or conflict.

22. The method of any one of embodiments 1-21, wherein the nucleic acid sample is derived from saliva, blood, semen, hair, teeth, bone, or skin.

23. The method of embodiment 22, wherein the nucleic acid sample is derived from saliva, blood, or semen.

24. The method of embodiment 22, wherein the nucleic acid sample is derived from bone or hair.

25. The method of any one of embodiments 1-21, wherein the nucleic acid sample is derived from a buccal swab, paper, fabric, or other substrate or object that is impregnated with saliva, blood, semen, or other bodily fluid.

26. The method of any one of embodiments 1-25, wherein the nucleic acid sample comprises between or between about 3 pg and 100 ng of genomic DNA.

27. The method of any one of embodiments 1-26, wherein the nucleic acid sample comprises between or between about 100 pg and 5 ng of genomic DNA, between or between about 50 pg and 5 ng of genomic DNA, or between or between about 3 pg and 5 ng of genomic DNA.

28. The method of embodiment 26 or embodiment 27, wherein the nucleic acid sample comprises at or about 1 ng of genomic DNA.

29. The method of any one of embodiments 1-28, wherein the plurality of SNPs comprises kinship SNPs (kiSNPs).

30. The method of any one of embodiments 1-29, wherein the plurality of SNPs comprises Y-chromosome SNPs (Y-SNPs).

31. The method of any one of embodiments 1-30, wherein the plurality of SNPs comprises kiSNPs and Y-SNPs.

32. The method of any one of embodiments 1-31, wherein the plurality of SNPs comprises kiSNPs, biogeographical ancestry SNPs (aiSNPs), identity SNPs (iiSNPs), phenotype SNPs (piSNPs), X-chromosome SNPs (X-SNPs), and Y-chromosome SNPs (Y-SNPs).

33. The method of any one of embodiments 1-28, wherein the plurality of SNPs comprises SNPs selected from one or more of the groups consisting of kiSNPs, aiSNPs, iiSNPs, piSNPs, X-SNPs, and Y-SNPs.

34. The method of any one of embodiments 1-33, wherein at least or at least about 80%, 81%, 82%, 83%, 84%, 85%, 86%, 87%, 88%, 89%, 90%, 91%, 92%, 93%, 94%, 95%, 96%, 97%, 98%, or 99% of the plurality of SNPs are kinship SNPs.

35. The method of any one of embodiments 1-34, wherein the reference set of DNA profiles comprises up to 5, 10, 15, 20, 25, 30, 35, 40, 45, 50, 55, 60, 65, 70, 75, 80, 85, 90, 95, 100, 125, 150, 175, 200, 300, 400, 500, 600, 700, 800, 900, or 1000 reference DNA profiles.

36. The method of any one of embodiments 1-35, wherein at least 5%, 10%, 15%, 20%, 25%, 30%, 35%, 40%, 45%, 50%, 55%, 60%, 65%, 70%, 75%, 80%, 85%, 90%, or 95% of the reference DNA profiles in the reference set of DNA profiles is from a relative of the person of interest.

37. The method of any one of embodiments 1-36, wherein at least 5%, 10%, 15%, 20%, 25%, 30%, 35%, 40%, 45%, 50%, 55%, 60%, 65%, 70%, 75%, 80%, 85%, 90%, or 95% of the reference DNA profiles in the reference set of DNA profiles is from a relative of the person of interest, and wherein each of the at least 5%, 10%, 15%, 20%, 25%, 30%, 35%, 40%, 45%, 50%, 55%, 60%, 65%, 70%, 75%, 80%, 85%, 90%, or 95% of the reference DNA profiles in the reference set of DNA profiles is a first degree, second degree, third degree, fourth degree, or fifth degree relative.

38. The method of any one of embodiments 1-37, wherein at least 50% of the reference DNA profiles in the reference set of DNA profiles is from a relative of the person of interest.

39. The method of any one of embodiments 36-38, wherein each relative of the person of interest in the reference set of DNA profiles is individually a first degree, second degree, third degree, fourth degree, or fifth degree relative of the person of interest.

40. The method of embodiment 39, wherein each relative of the person of interest in the reference set of DNA profiles is individually a first degree, second degree, or third degree relative of the person of interest.

41. The method of any one of embodiments 1-40, wherein the identity of each relative of the person of interest in the reference set of DNA profiles is known.

42. The method of any one of embodiments 1-41, wherein the identity of each of the one or more reference DNA profiles in the reference set of DNA profiles is known.

43. The method of any one of embodiments 1-42, wherein the reference set of DNA profiles is in a database.

44. The method of embodiment 43, wherein the database is not publicly accessible.

45. The method of any one of embodiments 1-44, wherein the sequencing comprises a sequencing plexity of up to 40-plex.

46. The method of any one of embodiments 1-44, wherein the sequencing comprises a sequencing plexity of up to 32-plex.

47. The method of any one of embodiments 1-44, wherein the sequencing comprises a sequencing plexity of 12-plex to 32-plex.

48. The method of any one of embodiments 1-44, wherein the sequencing comprises a sequencing plexity of 24-plex to 32-plex.

49. The method of any one of embodiments 1-44, wherein the sequencing comprises a sequencing plexity of at or about 10-plex, 11-plex, 12-plex, 13-plex, 14-plex, 15-plex, 16-plex, 17-plex 18-plex, 19-plex, 20-plex, 21-plex, 22-plex, 23-plex, 24-plex, 25-plex, 26-plex, 27-plex, 28-plex, 29-plex, 30-plex, 31-plex, 32-plex, 33-plex, 34-plex, or 35-plex.

50. The method of any one of embodiments 1-49, wherein the sequencing comprises a sequencing plexity of at or about 8-to 16-plex for postmortem samples, and/or the sequencing comprises a sequencing plexity of at or about 24-to 40-plex for antemortem samples.

51. The method of any one of embodiments 1-50, wherein the sequencing comprises a sequencing plexity of at or about 12-plex for postmortem samples, and/or the sequencing comprises a sequencing plexity of at or about 32-plex for antemortem samples.

52. The method of any one of embodiments 1-51, wherein the sequencing comprises a sequencing plexity of at or about 30-plex, 31-plex, or 32-plex.

53. The method of any one of embodiments 1-52, further comprising identifying the person of interest.

54. A method for calculating degree of relatedness, comprising:
obtaining a DNA profile comprising genotypes of at least between at or about 2,000 to 50,000 SNPs, wherein the DNA profile is from a person of interest; and
calculating the degree of relationship of the DNA profile to one or more reference DNA profiles, wherein the one or more reference DNA profiles are comprised within a reference set of DNA profiles comprising one or more reference DNA profiles from a relative of the person of interest.

55. A method for calculating degree of relatedness, comprising:
generating a DNA profile comprising genotypes of at least between at or about 2,000 to 50,000 SNPs, wherein the DNA profile is from a person of interest; and
calculating the degree of relationship of the DNA profile to one or more reference DNA profiles, wherein the one or more reference DNA profiles are comprised within a reference set of DNA profiles comprising one or more reference DNA profiles from a relative of the person of interest.

56. The method of any one of embodiments 1-55, wherein the degree of relationship is calculated using a kinship model.

57. The method of any one of embodiments 1-56, wherein the degree of relationship is calculated using a kinship model that is trained using a PCA method.

58. The method of embodiment 57, wherein the PCA method for training the kinship model is PCA or involves PCA.

59. The method of embodiment 57 or embodiment 58, wherein the PCA method is PC-AiR.

60. The method of embodiment 59, wherein the PC-AiR comprises the steps of: (1) estimating kinship coefficients between all pairs of samples, optionally training DNA profiles, of a training database, wherein pairings with a kinship coefficient >0.025 are identified as related and pairings with a kinship coefficient <−0.025 are identified as ancestry-diverged; (2) initializing an unrelated sample set that includes all samples; and (3) iteratively: (i) identifying the set in the unrelated sample set that have the most related samples in the unrelated sample set, thereby designated as X, (ii) identifying the set of samples in X that have the least ancestry-diverged pairings compared to samples in the unrelated sample set, thereby designated as Y; and (iii) if Y has zero samples, then terminating the process, or, if Y has at least one sample, then randomly selecting one sample from Y to remove from U, and repeat beginning at step (3) (i).

61. The method of embodiment 57 or embodiment 58, wherein the PCA method is a modified PC-Air.

62. The method of embodiment 61, wherein the modified PC-AiR comprises steps of: (1) estimating kinship coefficients between all pairs of samples, optionally training DNA profiles, of a training database, wherein pairings with a kinship coefficient >0.01 are identified as related and pairings with a kinship coefficient <−0.025 are identified as ancestry-diverged; (2) removing all DNA profiles that have ≥5% missing data; and (3) ranking all DNA profiles by identifying each DNA profile with a ranking value. In some embodiments, the ranking value is determined based on the number of related DNA profiles in the full database that is ranked from least to most, and ties are broken by the number of ancestry-diverged DNA profiles in the full database as ranked from most to least. In some embodiments, step (3) involves going iteratively through the ranked DNA profiles, and for each DNA profile: (i) if the DNA profile is not yet in the related sample set, adding it to the unrelated sample set and adding all related DNA profiles to the related sample set, and (ii) if the DNA profile is already in the related sample set, then skipping to the next DNA profile, and repeating beginning at step (3) (i).

63. The method of any one of embodiments 1-62, wherein the calculating the degree of relationship comprises calculating a kinship coefficient using PC-Relate.

64. The method of embodiment 63, wherein the degree of relationship is calculated by providing the DNA profile of the person of interest as input to PC-Relate.

65. The method of any one of embodiments 57-64, wherein the degree of relationship is calculated by providing the kinship model and the DNA profile of the person of interest as input to PC-Relate.

66. The method of any one of embodiments 63-65, wherein the one or more reference DNA profiles are further provided as input to PC-Relate.

67. The method of any one of embodiments 1-66, wherein the calculating the degree of relationship comprises calculating a kinship coefficient using a whole genome kinship alogirthm as follows:

$$\text{?} = \frac{{\sum }_{s ∈ S} ( {g}_{i s} - 2 {\hat{u}}_{i s} ) ( {g}_{j s} - 2 {\hat{u}}_{j s} )}{4 {{\sum }_{s ∈ S} [ {\hat{u}}_{i s} ( 1 - {\hat{u}}_{i s} ) \cdot {\hat{u}}_{j s} ( 1 - {\hat{u}}_{j s} ) ]}^{1 / 2}}$$

wherein the person of interest and a reference DNA profile of the one or more reference DNA profiles are i and j, [symbol] is the kinship coefficient, û is the estimated allele frequencies, s is a SNP in S SNPs that were typed in both individuals, gᵢₛ and gⱼₛ are the number of reference alleles in i and j at SNP s, respectively, and ûᵢₛ and ûⱼₛ are the expected allele frequencies calculated by PC-AiR for i and j at SNP s, respectively.

68. The method of any one of embodiments 1-67, wherein the calculating the degree of relationship comprises calculating a likelihood ratio.

69. The method of embodiment 68, wherein the calculating the likelihood ratio comprises comparing the plurality of SNPs between the DNA profile and the one or more reference DNA profiles.

70. The method of embodiment 68, wherein the calculating the likelihood ratio comprises comparing a set of SNPs comprising kinship SNPs from within the plurality of SNPs between the DNA profile and the one or more reference DNA profiles.

71. The method of any one of embodiments 68-70, wherein calculating the likelihood ratio comprises dividing the probability of the DNA profile and a reference DNA profile from among the one or more reference DNA profiles being related by the probability of the DNA profile and the reference DNA profile being unrelated based on the genotypes of the plurality of SNPs.

72. The method of any one of embodiments 68-71, wherein the likelihood ratio (LR) is calculated as follows:

$$L R = \frac{P ( D \vert {H}_{r} )}{P ( D \vert {H}_{u} )}$$

wherein D represents the genotypes, Hᵣ represents the hypothesis that the individuals are related, and Hᵤ represents the hypothesis that the individuals are unrelated.

73. The method of any one of embodiments 68-71, wherein the LR is calculated as as follows:

$$L R = \frac{0.001}{{p}^{2} {q}^{2}}$$

wherein 0.001 represents a genotyping error rate, p is the allele frequency of the allele 1, and q is the allele frequency of allele 2.

74. The method of any one of embodiments 1-73, wherein the person of interest is biologically male and the method further comprises calculating a likelihood ratio for sharing a Y chromosome between the DNA profile and the one or more reference DNA profiles.

75. The method of embodiment 74, wherein the calculating a likelihood ratio for sharing a Y chromosome comprises comparing a set of SNPs that comprises one or more Y-SNPs between the DNA profile and the one or more reference DNA profiles.

76. The method of embodiment 75, wherein the one or more Y-SNPs are comprised within the plurality of SNPs.

77. The method of embodiment 75, wherein the one or more Y-SNPs comprises at least 20, 25, 30, 35, 40, 45, 50, 55, 60, 65, 70, 75, 80, 81, 82, 83, 84, or 85 Y-SNPs.

78. The method of any one of embodiments 75-77, wherein the one or more Y-SNPs comprises 85 Y-SNPs.

79. The method of any one of embodiments 75-78, wherein calculating the likelihood ratio for sharing a Y chromosome comprises dividing the probability of the DNA profile and a reference DNA profile from among the one or more reference DNA profiles sharing a Y chromosome by the probability of the DNA profile and the reference DNA profile not sharing a Y chromosome based on the genotypes of the one or more Y-SNPs.

80. The method of any one of embodiments 1-79, wherein at least 90%, 91%, 92%, 93%, 94%, 95%, 96%, 97%, 98%, or 99% of the DNA profiles in the reference set of DNA profiles is from a relative of a missing person or a victim of a disaster or a conflict.

81. The method of any one of embodiments 1-80, wherein each of the DNA profiles in the reference set of DNA profiles is from a relative of a missing person or a victim of a disaster or a conflict.

82. The method of any one of embodiments 1-81, wherein the reference set of DNA profiles comprises up to 5, 10, 15, 20, 25, 30, 35, 40, 45, 50, 55, 60, 65, 70, 75, 80, 85, 90, 95, 100, 125, 150, 175, 200, 300, 400, 500, 600, 700, 800, 900, or 1000 reference DNA profiles.

83. The method of any one of embodiments 1-82, wherein the reference set of DNA profiles comprises up to 100 reference DNA profiles.

84. The method of any one of embodiments 1-83, wherein at least 5%, 10%, 15%, 20%, 25%, 30%, 35%, 40%, 45%, 50%, 55%, 60%, 65%, 70%, 75%, 80%, 85%, 90%, or 95% of the reference DNA profiles in the reference set of DNA profiles is from a relative of the person of interest. 85. The method of any one of embodiments 1-84, wherein at least 50% of the reference DNA profiles in the reference set of DNA profiles is from a relative of the person of interest.

86. The method of embodiment 84 or embodiment 85, wherein each relative of the person of interest in the reference set of DNA profiles is individually a first degree, second degree, third degree, fourth degree, or fifth degree relative of the person of interest.

87. The method of any one of embodiments 1-86, wherein the identity of each relative of the person of interest in the reference set of DNA profiles is known.

88. The method of any one of embodiments 1-87, wherein the identity of each relative of the person of interest in the reference set of DNA profiles is known.

89. The method of any one of embodiments 1-88, wherein the identity of each of the one or more reference DNA profiles in the reference set of DNA profiles is known.

90. The method of any one of embodiments 1-89, wherein the reference set of DNA profiles is in a database.

91. The method of embodiment 90, wherein the database is not publicly accessible.

92. The method of embodiment 90 or embodiment 91, wherein the database is not accessible by a third party geneaological service.

93. A nucleic acid library constructed using the method of any one of embodiments 6-92.

94. A plurality of primers that specifically hybridize to a plurality of target sequences comprising at least between at or about 2,000 to 50,000 single nucleotide polymorphisms (SNPs) in a nucleic acid sample from a person of interest, wherein amplifying the nucleic acid sample using the plurality of primers in one or more multiplex PCR reactions results in amplification products.

95. A plurality of primers that specifically hybridize to a plurality of target sequences comprising at least between at or about 2,000 to 50,000 single nucleotide polymorphisms (SNPs) in a nucleic acid sample from a person of interest and a nucleic acid sample from one or more reference samples,
wherein the one or more reference samples comprises a sample from a relative of the person of interest, and
wherein amplifying the nucleic acid sample from the person of interest and the nucleic acid sample from one or more reference samples using the plurality of primers in one or more multiplex PCR reactions results in amplification products.

96. The plurality of primers of embodiment 94 or embodiment 95, wherein the nucleic acid sample from the person of interest comprises genomic DNA.

97. The plurality of primers of any one of embodiments 94-96, wherein the nucleic acid sample from the person of interest comprises one or more enzyme inhibitors.

98. The plurality of primers of embodiment 97, wherein the one or more enzyme inhibitors comprise one or more inhibitors selected from the group consisting of hematin, heme, humic acid, indigo, tannic acid, collagen, calcium, and hydroxyapatite.

99. The plurality of primers of any one of embodiments 94-98, wherein the nucleic acid sample from the person of interest comprises low-quality nucleic acid molecules and/or low quantity nucleic acid molecules.

100. The plurality of primers of embodiment 99, wherein the low quality nucleic acid molecules are degraded genomic DNA and/or fragmented genomic DNA.

101. The plurality of primers of embodiment 99 or embodiment 100, wherein the low quality nucleic acid molecules have a degradation index (DI) of at or at least 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200.

102. The plurality of primers of any one of embodiments 99-101, wherein the low quality nucleic acid molecules have a DI of at least 1 and up to or less than 158.3.

103. The plurality of primers of any one of embodiments 94-102, wherein the nucleic acid sample from the person of interest and/or the nucleic acid sample from one or more reference samples comprises high quality nucleic acid molecules.

104. The plurality of primers of embodiment 103, wherein the high quality nucleic acid molecules have a DI of less than 1.

105. The plurality of primers of any one of embodiments 94-104, wherein the person of interest is a missing person.

106. The plurality of primers of any one of embodiments 94-104, wherein the person of interest is a victim of a disaster or conflict.

107. The plurality of primers of any one of embodiments 94-106, wherein the nucleic acid sample from the person of interest is derived from a buccal swab, paper, fabric, or other substrate or object that is impregnated with saliva, blood, or other bodily fluid, or contains hair or skin cells. 108. The plurality of primers of any one of embodiments 94-107, wherein the nucleic acid sample from the person of interest comprises between or between about 3 pg and 100 ng of genomic DNA.

109. The plurality of primers of any one of embodiments 94-108, wherein the nucleic acid sample from the person of interest comprises between or between about 100 pg and 5 ng of genomic DNA, between or between about 50 pg and 5 ng of genomic DNA, or between or between about 3 pg and 5 ng of genomic DNA.

110. The plurality of primers of embodiment 108 or embodiment 109, wherein the nucleic acid sample from the person of interest comprises at or about 1 ng of genomic DNA.

111. The plurality of primers of any one of embodiments 94-110, wherein the plurality of SNPs comprises kinship SNPs (kiSNPs).

112. The method of any one of embodiments 94-111, wherein the plurality of SNPs comprises Y-chromosome SNPs (Y-SNPs).

113. The method of any one of embodiments 94-112, wherein the plurality of SNPs comprises kiSNPs and Y-SNPs.

114. The plurality of primers of any one of embodiments 94-113, wherein the plurality of SNPs comprises kiSNPs, biogeographical ancestry SNPs (aiSNPs), identity SNPs (iiSNPs), phenotype SNPs (piSNPs), X-chromosome SNPs (X-SNPs), and Y-chromosome SNPs (Y-SNPs).

115. The plurality of primers of any one of embodiments 94-111, wherein the plurality of SNPs comprises SNPs selected from one or more of the groups consisting of kiSNPs, aiSNPs, iiSNPs, piSNPs, X-SNPs, and Y-SNPs.

116. The plurality of primers of any one of embodiments 94-115, wherein at least or at least about 80%, 81%, 82%, 83%, 84%, 85%, 86%, 87%, 88%, 89%, 90%, 91%, 92%, 93%, 94%, 95%, 96%, 97%, 98%, or 99% of the plurality of SNPs are kinship SNPs.

117. The plurality of primers of any one of embodiments 94-116, wherein at least 90%, 91%, 92%, 93%, 94%, 95%, 96%, 97%, 98%, or 99% of the DNA profiles in the reference set of DNA profiles is from a relative of a missing person or a victim of a disaster or a conflict.

118. The plurality of primers of any one of embodiments 94-116, wherein each of the one or more references samples is from a relative of a missing person or a victim of a disaster or a conflict.

119. The plurality of primers of any one of embodiments 94-118, wherein the one or more reference samples comprises up to 5, 10, 15, 20, 25, 30, 35, 40, 45, 50, 55, 60, 65, 70, 75, 80, 85, 90, 95, 100, 125, 150, 175, 200, 300, 400, 500, 600, 700, 800, 900, or 1000 reference samples.

120. The plurality of primers of any one of embodiments 94-119, wherein at least 5%, 10%, 15%, 20%, 25%, 30%, 35%, 40%, 45%, 50%, 55%, 60%, 65%, 70%, 75%, 80%, 85%, 90%, or 95% of the one or more reference samples is from a relative of the person of interest.

121. The plurality of primers of any one of embodiments 94-120, wherein at least 5%, 10%, 15%, 20%, 25%, 30%, 35%, 40%, 45%, 50%, 55%, 60%, 65%, 70%, 75%, 80%, 85%, 90%, or 95% of the reference DNA profiles in the reference set of DNA profiles is from a relative of the person of interest, and wherein each of the at least 5%, 10%, 15%, 20%, 25%, 30%, 35%, 40%, 45%, 50%, 55%, 60%, 65%, 70%, 75%, 80%, 85%, 90%, or 95% of the reference DNA profiles in the reference set of DNA profiles is a first degree, second degree, third degree, fourth degree, or fifth degree relative.

122. The plurality of primers of any one of embodiments 94-121, wherein at least 50% of the one or more reference samples is from a relative of the person of interest.

123. The plurality of primers of any one of embodiments 120-122, wherein each relative of the person of interest in the one or more reference samples is individually a first degree, second degree, third degree, fourth degree, or fifth degree relative of the person of interest.

124. The plurality of primers of embodiment 123, wherein each relative of the person of interest in the one or more reference samples is individually a first degree, second degree, or third degree relative of the person of interest.

125. The plurality of primers of any one of embodiments 94-124, wherein the identity of each relative of the person of interest in the one or more reference samples is known.

126. The plurality of primers of any one of embodiments 94-125, wherein the identity of each of the one or more reference samples is known.

127. A method for constructing a DNA profile, comprising:
providing a nucleic acid sample from a person of interest,
amplifying the nucleic acid sample with a plurality of primers that specifically hybridize to a plurality of target sequences collectively comprising a plurality of at least between at or about 2,000 to 50,000 single nucleotide polymorphisms (SNPs), thereby generating amplification products, wherein the amplification is carried out in one or more multiplex PCR reactions,
sequencing the amplification products,
determining the genotypes of the plurality of SNPs, thereby generating a DNA profile.

128. A method for constructing a DNA profile, comprising:
providing a nucleic acid sample from a person of interest,
providing a nucleic acid sample from a relative of the person of interest,
amplifying the nucleic acid sample from the person of interest and the nucleic acid sample from the relative with a plurality of primers that specifically hybridize to a plurality of target sequences collectively comprising a plurality of at least between at or about 2,000 to 50,000 single nucleotide polymorphisms (SNPs), thereby generating amplification products, wherein the amplification is carried out in one or more multiplex PCR reactions,
sequencing the amplification products,
determining the genotypes of the plurality of SNPs, thereby generating a DNA profile for the person of interest and the relative of the person of interest.

129. The method of embodiment 127 or embodiment 128, wherein the sequencing does not comprise whole genome sequencing (WGS).

130. The method of embodiment 127 or embodiment 129, wherein the nucleic acid sample comprises genomic DNA.

131. The method of embodiment 128 or embodiment 129, wherein the nucleic acid sample of the person of interest and/or the nucleic acid sample of the relative of the person of interest comprises genomic DNA.

132. The method of any one of embodiments 127-131, wherein the nucleic acid sample, the nucleic acid sample of the person of interest, and/or the nucleic acid sample of the relative comprises one or more enzyme inhibitors.

133. The method of embodiment 132, wherein the one or more enzyme inhibitors comprise one or more inhibitors selected from the group consisting of hematin, heme, humic acid, indigo, tannic acid, collagen, calcium, and hydroxyapatite.

134. The method of any one of embodiments 127-133, wherein the nucleic acid sample, the nucleic acid sample of the person of interest, and/or the nucleic acid sample of the relative comprises low-quality nucleic acid molecules and/or low quantity nucleic acid molecules.

135. The method of embodiment 134, wherein the low quality nucleic acid molecules are degraded genomic DNA and/or fragmented genomic DNA.

136. The method of embodiment 134 or embodiment 135, wherein the low quality nucleic acid molecules have a degradation index (DI) of at or at least 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200.

137. The method of any one of embodiments 134-136, wherein the low quality nucleic acid molecules have a DI of at least 1 and up to or less than 158.3.

138. The method of any one of embodiments 127-133, wherein the nucleic acid sample, the nucleic acid sample of the person of interest, and/or the nucleic acid sample of the relative comprises high quality nucleic acid molecules.

139. The method of embodiment 138, wherein the high quality nucleic acid molecules have a DI of less than 1.

140. The method of any one of embodiments 127-139, wherein the person of interest is a missing person.

141. The method of any one of embodiments 127-139, wherein the person of interest is a victim of a disaster or conflict.

142. The method of any one of embodiments 128, 129, and 131-141, wherein the relative of the person of interest is a first-, second-, third-, fourth-, or fifth-degree relative.

143. The method of any one of embodiments 128, 129, and 131-141, wherein the relative of the person of interest is a first-, second-, or third-degree relative.

144. The method of any one of embodiments 127-143, wherein the nucleic acid sample, the nucleic acid sample of the person of interest, and/or the nucleic acid sample of the relative is derived from a buccal swab, paper, fabric, or other substrate or object that is impregnated with saliva, blood, or other bodily fluid, or contains hair or skin cells.

145. The method of any one of embodiments 127-144, wherein the nucleic acid sample, the nucleic acid sample of the person of interest, and/or the nucleic acid sample of the relative comprises between or between about 3 pg and 100 ng of genomic DNA.

146. The method of any one of embodiments 127-145, wherein the nucleic acid sample, the nucleic acid sample of the person of interest, and/or the nucleic acid sample of the relative comprises between or between about 100 pg and 5 ng of genomic DNA, between or between about 50 pg and 5 ng of genomic DNA, or between or between about 3 pg and 5 ng of genomic DNA.

147. The method of embodiment 145 or embodiment 146, wherein the nucleic acid sample, the nucleic acid sample of the person of interest, and/or the nucleic acid sample of the relative comprises at or about 1 ng of genomic DNA.

148. The method of any one of embodiments 127-147 wherein the plurality of SNPs comprises kinship SNPs.

149. The method of any one of embodiments 127-148, wherein the plurality of SNPs comprises Y-chromosome SNPs (Y-SNPs).

150. The method of any one of embodiments 127-149, wherein the plurality of SNPs comprises kiSNPs and Y-SNPs.

151. The method of any one of embodiments 127-150, wherein the plurality of SNPs comprises kiSNPs, biogeographical ancestry SNPs (aiSNPs), identity SNPs (iiSNPs), phenotype SNPs (piSNPs), X-chromosome SNPs (X-SNPs), and Y-chromosome SNPs (Y-SNPs).

152. The method of any one of embodiments 127-151, wherein the plurality of SNPs comprises SNPs selected from one or more of the groups consisting of kiSNPs, aiSNPs, iiSNPs, piSNPs, X-SNPs, and Y-SNPs.

153. The method of any one of embodiments 127-152, wherein at least or at least about 80%, 81%, 82%, 83%, 84%, 85%, 86%, 87%, 88%, 89%, 90%, 91%, 92%, 93%, 94%, 95%, 96%, 97%, 98%, or 99% of the plurality of SNPs are kinship SNPs.

154. The method of any one of embodiments 1-92 and 127-153, wherein the sequencing comprises a sequencing plexity of up to 40-plex.

155. The method of any one of embodiments 1-92 and 127-153, wherein the sequencing comprises a sequencing plexity of up to 32-plex.

156. The method of any one of embodiments 1-92 and 127-153, wherein the sequencing comprises a sequencing plexity of 12-plex to 32-plex.

157. The method of any one of embodiments 1-92 and 127-153, wherein the sequencing comprises a sequencing plexity of 24-plex to 32-plex.

158. The method of any one of embodiments 1-92 and 127-153, wherein: (a) the sequencing comprises a sequencing plexity of at or about 4-plex, 5-plex, 6-plex, 7-plex, 8-plex, 9-plex, 10-plex, 11-plex, 12-plex, 13-plex, 14-plex, 15-plex, 16-plex, 17-plex 18-plex, 19-plex, 20-plex, 21-plex, 22-plex, 23-plex, 24-plex, 25-plex, 26-plex, 27-plex, 28-plex, 29-plex, 30-plex, 31-plex, 32-plex, 33-plex, 34-plex, 35-plex, 36-plex, 37-plex, 38-plex, 39-plex, 40-plex, 41-plex, 42-plex, 43-plex, 44-plex, or 45-plex; or (b) the sequencing comprises a sequencing plexity of at or about 10-plex, 11-plex, 12-plex, 13-plex, 14-plex, 15-plex, 16-plex, 17-plex 18-plex, 19-plex, 20-plex, 21-plex, 22-plex, 23-plex, 24-plex, 25-plex, 26-plex, 27-plex, 28-plex, 29-plex, 30-plex, 31-plex, 32-plex, 33-plex, 34-plex, or 35-plex.

159. The method of any one of embodiments 1-92 and 127-158, wherein the sequencing comprises a sequencing plexity of at or about 8-to 16-plex for postmortem samples, and/or the sequencing comprises a sequencing plexity of at or about 24-to 40-plex for antemortem samples.

160. The method of any one of embodiments 1-92 and 127-158, wherein the sequencing comprises a sequencing plexity of at or about 12-plex for postmortem samples, and/or the sequencing comprises a sequencing plexity of at or about 32-plex for antemortem samples.

161. The method of any one of embodiments 1-92 and 127-153, wherein the sequencing comprises a sequencing plexity of at or about 30-plex, 31-plex, or 32-plex.

162. A method of identifying genetic relatives of a DNA profile, comprising:
calculating the degree of relationship of the DNA profile of any one of embodiments 127-161 to one or more reference DNA profiles, wherein the one or more reference DNA profiles are comprised within a reference set of DNA profiles comprising one or more reference DNA profiles from a relative of the person of interest; and
generating a family tree comprising the DNA profile in relation to the one or more reference DNA profiles.

163. The method of embodiment 162, wherein the one or more reference DNA profiles are part of a database.

164. The method of embodiment 162 or embodiment 163, wherein the reference set of DNA profiles comprises up to 5, 10, 15, 20, 25, 30, 35, 40, 45, 50, 55, 60, 65, 70, 75, 80, 85, 90, 95, 100, 125, 150, 175, 200, 300, 400, 500, 600, 700, 800, 900, or 1000 reference DNA profiles.

165. The method of any one of embodiments 162-164, wherein at least 5%, 10%, 15%, 20%, 25%, 30%, 35%, 40%, 45%, 50%, 55%, 60%, 65%, 70%, 75%, 80%, 85%, 90%, or 95% of the reference DNA profiles in the reference set of DNA profiles is from a relative of the person of interest.

166. The method of any one of embodiments 162-165, wherein at least 50% of the reference DNA profiles in the reference set of DNA profiles is from a relative of the person of interest.

167. The method of embodiment 165 or embodiment 166, wherein each relative of the person of interest is individually a first degree, second degree, third degree, fourth degree, or fifth degree relative of the person of interest.

168. The method of any one of embodiments 162-167, wherein the identity of each relative of the person of interest in the reference set of DNA profiles is known.

169. The method of any one of embodiments 162-168, wherein the identity of each of the one or more reference DNA profiles in the reference set of DNA profiles is known.

170. The method of any one of embodiments 162-169, wherein the reference set of DNA profiles is in a database.

171. The method of embodiment 170, wherein the database is not publicly accessible.

172. The method of embodiment 170 or embodiment 171, wherein the database is not accessible by a third party geneaological service.

173. A method of identifying the identity of a DNA profile, comprising:
calculating the degree of relationship of a DNA profile comprising genotypes of at least between at or about 2,000 to 50,000 SNPs to one or more reference DNA profiles, wherein the DNA profile is from a person of interest, and wherein the one or more reference DNA profiles are comprised within a reference set of DNA profiles comprising one or more reference DNA profiles from a relative of the person of interest; and
generating a family tree comprising the DNA profile in relation to the one or more reference DNA profiles.

174. The method of embodiment 173, wherein the DNA profile is generated by the method of any one of embodiments 127-161.

175. The method of embodiment 173 or embodiment 174, wherein the degree of relationship is calculated using a kinship model.

176. The method of any one of embodiments 173-175, wherein the degree of relationship is calculated using a kinship model that is trained using a PCA method.

177. The method of embodiment 176, wherein the PCA method for training the kinship model is PCA or involves PCA.

178. The method of embodiment 176 or embodiment 177, wherein the PCA method is PC-AiR.

179. The method of embodiment 178, wherein the PC-AiR comprises the steps of: (1) estimating kinship coefficients between all pairs of samples, optionally training DNA profiles, of a training database, wherein pairings with a kinship coefficient >0.025 are identified as related and pairings with a kinship coefficient <−0.025 are identified as ancestry-diverged; (2) initializing an unrelated sample set that includes all samples; and (3) iteratively: (i) identifying the set in the unrelated sample set that have the most related samples in the unrelated sample set, thereby designated as X, (ii) identifying the set of samples in X that have the least ancestry-diverged pairings compared to samples in the unrelated sample set, thereby designated as Y; and (iii) if Y has zero samples, then terminating the process, or, if Y has at least one sample, then randomly selecting one sample from Y to remove from U, and repeat beginning at step (3) (i).

180. The method of embodiment 176 or embodiment 177, wherein the PCA method is a modified PC-Air.

181. The method of embodiment 180, wherein the modified PC-AiR comprises steps of: (1) estimating kinship coefficients between all pairs of samples, optionally training DNA profiles, of a training database, wherein pairings with a kinship coefficient >0.01 are identified as related and pairings with a kinship coefficient <−0.025 are identified as ancestry-diverged; (2) removing all DNA profiles that have ≥5% missing data; and (3) ranking all DNA profiles by identifying each DNA profile with a ranking value. In some embodiments, the ranking value is determined based on the number of related DNA profiles in the full database that is ranked from least to most, and ties are broken by the number of ancestry-diverged DNA profiles in the full database as ranked from most to least. In some embodiments, step (3) involves going iteratively through the ranked DNA profiles, and for each DNA profile: (i) if the DNA profile is not yet in the related sample set, adding it to the unrelated sample set and adding all related DNA profiles to the related sample set, and (ii) if the DNA profile is already in the related sample set, then skipping to the next DNA profile, and repeating beginning at step (3) (i).

182. The method of any one of embodiments 173-181, wherein the calculating the degree of relationship comprises calculating a kinship coefficient using PC-Relate.

183. The method of embodiment 182, wherein the degree of relationship is calculated by providing the DNA profile of the person of interest as input to PC-Relate.

184. The method of embodiment 182 or embodiment 183, wherein the degree of relationship is calculated by providing the kinship model and the DNA profile of the person of interest as input to PC-Relate.

185. The method of any one of embodiments 182-184, wherein the one or more reference DNA profiles are further provided as input to PC-Relate.

186. The method of any one of embodiments 173-185, wherein the calculating the degree of relationship comprises calculating a kinship coefficient using a whole genome kinship alogirthm as follows:

$$\text{?} = \frac{{\sum }_{s ∈ S} ( {g}_{i s} - 2 {\hat{u}}_{i s} ) ( {g}_{j s} - 2 {\hat{u}}_{j s} )}{4 {{\sum }_{s ∈ S} [ {\hat{u}}_{i s} ( 1 - {\hat{u}}_{i s} ) \cdot {\hat{u}}_{j s} ( 1 - {\hat{u}}_{j s} ) ]}^{1 / 2}}$$

wherein the person of interest and a reference DNA profile of the one or more reference DNA profiles are i and j, [symbol] is the kinship coefficient, û is the estimated allele frequencies, s is a SNP in S SNPs that were typed in both individuals, gᵢₛ and gⱼₛ are the number of reference alleles in i and j at SNP s, respectively, and ûᵢₛ and ûⱼₛ are the expected allele frequencies calculated by PC-AiR for i and j at SNP s, respectively.

187. The method of any one of embodiments 173-186, wherein the calculating the degree of relationship comprises calculating a likelihood ratio.

188. The method of embodiment 187, wherein the calculating the likelihood ratio comprises comparing the plurality of SNPs between the DNA profile and the one or more reference DNA profiles.

189. The method of embodiment 187, wherein the calculating the likelihood ratio comprises comparing a set of SNPs comprising kinship SNPs from within the plurality of SNPs between the DNA profile and the one or more reference DNA profiles.

190. The method of any one of embodiments 187-189, wherein calculating the likelihood ratio comprises dividing the probability of the DNA profile and a reference DNA profile from among the one or more reference DNA profiles being related by the probability of the DNA profile and the reference DNA profile being unrelated based on the genotypes of the plurality of SNPs.

191. The method of any one of embodiments 187-190, wherein the likelihood ratio (LR) is calculated as follows:

$$L R = \frac{P ( D \vert {H}_{r} )}{P ( D \vert {H}_{u} )}$$

wherein D represents the genotypes, Hᵣ represents the hypothesis that the individuals are related, and Hᵤ represents the hypothesis that the individuals are unrelated.

192. The method of any one of embodiments 187-190, wherein the LR is calculated as as follows:

$$L R = \frac{0.001}{{p}^{2} {q}^{2}}$$

wherein 0.001 represents a genotyping error rate, p is the allele frequency of the allele 1, and q is the allele frequency of allele 2.

193. The method of any one of embodiments 173-192, wherein the person of interest is biologically male and the method further comprises calculating a likelihood ratio for sharing a Y chromosome between the DNA profile and the one or more reference DNA profiles.

194. The method of embodiment 193, wherein the calculating a likelihood ratio for sharing a Y chromosome comprises comparing a set of SNPs that comprises one or more Y-SNPs between the DNA profile and the one or more reference DNA profiles.

195. The method of embodiment 194, wherein the one or more Y-SNPs are comprised within the plurality of SNPs.

196. The method of embodiment 194 or embodiment 195, wherein the one or more Y-SNPs comprises at least 20, 25, 30, 35, 40, 45, 50, 55, 60, 65, 70, 75, 80, 81, 82, 83, 84, or 85 Y-SNPs.

197. The method of any one of embodiments 194-196, wherein the one or more Y-SNPs comprises 85 Y-SNPs.

198. The method of any one of embodiments 193-197, wherein calculating the likelihood ratio for sharing a Y chromosome comprises dividing the probability of the DNA profile and a reference DNA profile from among the one or more reference DNA profiles sharing a Y chromosome by the probability of the DNA profile and the reference DNA profile not sharing a Y chromosome based on the genotypes of the one or more Y-SNPs.

199. The method of any one of embodiments 173-198, wherein at least 90%, 91%, 92%, 93%, 94%, 95%, 96%, 97%, 98%, or 99% of the DNA profiles in the reference set of DNA profiles is from a relative of a missing person or a victim of a disaster or a conflict.

200. The method of any one of embodiments 173-199, wherein each of the one or more references samples is from a relative of a missing person or a victim of a disaster or a conflict.

201. The method of any one of embodiments 173-200, wherein the one or more reference samples comprises up to 5, 10, 15, 20, 25, 30, 35, 40, 45, 50, 55, 60, 65, 70, 75, 80, 85, 90, 95, 100, 125, 150, 175, 200, 300, 400, 500, 600, 700, 800, 900, or 1000 reference samples.

202. The method of any one of embodiments 173-201, wherein at least 5%, 10%, 15%, 20%, 25%, 30%, 35%, 40%, 45%, 50%, 55%, 60%, 65%, 70%, 75%, 80%, 85%, 90%, or 95% of the one or more reference samples is from a relative of the person of interest.

203. The method of any one of embodiments 173-202, wherein at least 5%, 10%, 15%, 20%, 25%, 30%, 35%, 40%, 45%, 50%, 55%, 60%, 65%, 70%, 75%, 80%, 85%, 90%, or 95% of the reference DNA profiles in the reference set of DNA profiles is from a relative of the person of interest, and wherein each of the at least 5%, 10%, 15%, 20%, 25%, 30%, 35%, 40%, 45%, 50%, 55%, 60%, 65%, 70%, 75%, 80%, 85%, 90%, or 95% of the reference DNA profiles in the reference set of DNA profiles is a first degree, second degree, third degree, fourth degree, or fifth degree relative.

204. The method of any one of embodiments 173-203, wherein at least 50% of the one or more reference samples is from a relative of the person of interest.

205. The method of any one of embodiments 199-204, wherein each relative of the person of interest in the one or more reference samples is individually a first degree, second degree, third degree, fourth degree, or fifth degree relative of the person of interest.

206. The method of embodiment 205, wherein each relative of the person of interest in the one or more reference samples is individually a first degree, second degree, or third degree relative of the person of interest.

207. The method of any one of embodiments 173-206, wherein the identity of each relative of the person of interest in the one or more reference samples is known.

208. The method of any one of embodiments 173-207, wherein the identity of each of the one or more reference samples is known.

209. A kit comprising at least one container means, wherein the at least one container means comprises a plurality of primers of any one of embodiments 94-126.

210. The method of any one of embodiments 1-92 and 127-208, wherein the plurality of SNPs comprises between or between about 2,000 to 11,000 SNPs, 3,000 to 11,000 SNPs, 4,000 to 11,000 SNPs, 5,000 to 11,000 SNPs, 5,500 to 11,000 SNPs, 6,000 to 11,000 SNPs, 7,000 to 15,000 SNPs, 7,000 to 14,000 SNPs, 7,000 to 13,000 SNPs, 7,000 to 12,000 SNPs, 7,000 to 11,000 SNPs, 8,000 to 15,000 SNPs, 8,000 to 14,000 SNPs, 8,000 to 13,000 SNPs, 8,000 to 12,000 SNPs, 8,000 to 11,000 SNPs, 9,000 to 15,000 SNPs, 9,000 to 14,000 SNPs, 9,000 to 13,000 SNPs, 9,000 to 12,000 SNPs, or 9,000 to 11,000 SNPs.

211. The method of any one of embodiments 1-92 and 127-208, wherein the plurality of SNPs comprises 10,230 SNPs.

212. The plurality of primers of any one of embodiments 94-126, wherein the plurality of SNPs comprises between or between about 2,000 to 11,000 SNPs, 3,000 to 11,000 SNPs, 4,000 to 11,000 SNPs, 5,000 to 11,000 SNPs, 5,500 to 11,000 SNPs, 6,000 to 11,000 SNPs, 7,000 to 15,000 SNPs, 7,000 to 14,000 SNPs, 7,000 to 13,000 SNPs, 7,000 to 12,000 SNPs, 7,000 to 11,000 SNPs, 8,000 to 15,000 SNPs, 8,000 to 14,000 SNPs, 8,000 to 13,000 SNPs, 8,000 to 12,000 SNPs, 8,000 to 11,000 SNPs, 9,000 to 15,000 SNPs, 9,000 to 14,000 SNPs, 9,000 to 13,000 SNPs, 9,000 to 12,000 SNPs, or 9,000 to 11,000 SNPs.

213. The plurality of primers of any one of embodiments 94-126, wherein the plurality of SNPs comprises 10,230 SNPs.

214. The method of any one of embodiments 1-5, 11-93, 127-161, and 210-213,, further comprising generating a family tree comprising the DNA profile in relation to one or more DNA profiles comprised in the reference set of DNA profiles.

215. The method of embodiment 214, wherein the family tree comprises the DNA profile in relation to one or more DNA profiles from a relative of the person of interest.

### EXAMPLES

The following examples are included for illustrative purposes only and are not intended to limit the scope of the invention.

### Example 1: Generation of Sequence Libraries and Determination of Sensitivity

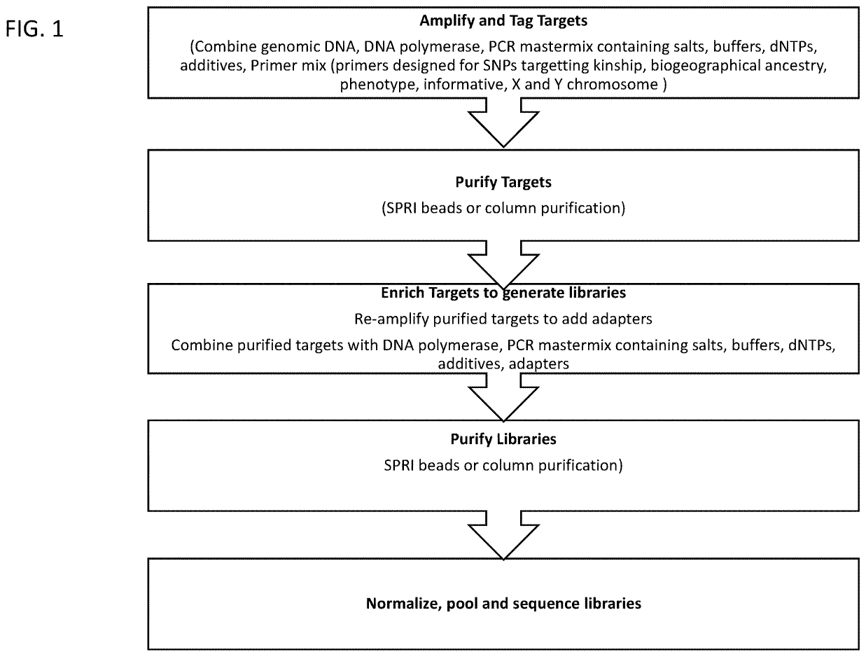

This Example describes a method of determining the sensitivity of the multiplex polymerase chain reaction described herein to generate libraries capable of being sequenced. FIG. 1 depicts an exemplary schematic of the method for generating a library capable of being sequenced described in this Example.

### A. PCR Amplification of Genomic DNA Target

A multiplex polymerase chain reaction was performed to amplify 10,230 individual amplicons in a genomic DNA sample. Each primer pair was designed to selectively hybridize to, and promote amplification of a specific single nucleotide polymorphism (SNP) of the genomic DNA sample. A range of input genomic DNA was tested from 50 ng to 50 pg, more specifically, 5 ng, 2.5 ng, 1 ng, 500 pg, 250 pg, 100 pg and 50 pg). Briefly, 18.5 ml of a PCR mastermix containing sufficient buffer, dNTPs, MgCl2, salts and PCR additives such as glycerol was added to a single well of a 96-well PCR plate. 5 microliters of Primer Pool, containing 10,530 primer pairs, 2-4 Units of a DNA polymerase such as Phusion hot start DNA polymerase (Thermo Fisher, cat #F549L or any other thermostable DNA polymerase, 50 ng to 50 pg genomic DNA were also added.

The PCR plate was sealed and loaded into a thermal cycler (Veriti 96-well thermal cycler, Thermo Fisher Scientific, 4413964) and run on the temperate profile described below to generate the amplicon library.
98 °C for 3 minutes
18 cycles of:
96 °C for 45 seconds
80 °C for 10 seconds
54 °C for 4 minutes with applicable ramp mode
66 °C for 90 seconds with applicable ramp mode
68 °C for 10 minutes
Hold at 4 °C.

After cycling, the amplicon library was held at 2-8 °C until proceeding to the purification step outlined below.

B. Purification of Amplicons from Input DNA and Primers

Two rounds of clean-up using MagBind Total Pure NGS beads (Omega Biotek, M1378-02) binding, wash, and elution at 1.6× and 0.6× volume ratios were found to remove genomic DNA and unbound or excess primers. The amplification and purification step outlined herein produces amplicons of about 150-350 bp in length. Purified amplicons are then used in a second round of PCR to add adapters for sequencing.

C. Enrichment of Purified Amplicons to Generate Libraries Capable of being Sequenced

A second round of PCR amplification is performed by combining 25 ml of purified amplicons from step above with 5 ml of adapters provided in Forenseq Kintelligence kit (Verogen PN: V16000120) and 20 ml of KPCR2 mastermix provided in Forenseq Kintelligence kit (Verogen PN: V16000120) in a 96 well PCR plate. The PCR plate was sealed and loaded into a thermal cycler (Veriti 96-well thermal cycler, Thermo Fisher Scientific, 4413964) and run on the temperate profile described below to generate the amplicon library.
98 °C for 30 seconds
15 cycles of:
98 °C for 20 seconds
66 °C for 30 seconds
72 °C for 30 seconds
72 °C for 1 minute
Hold at 4 °C.

The libraries were purified using MagBind Total Pure NGS beads (Omega Biotek, M1378-02) binding, wash, and elution at 1×. The purified libraries were quantitated, normalized, denatured and diluted as per instructions given in Forenseq Kintelligence kit User Guide (Verogen PN: V16000120, the contents of which are hereby incorporated by reference in their entirety).

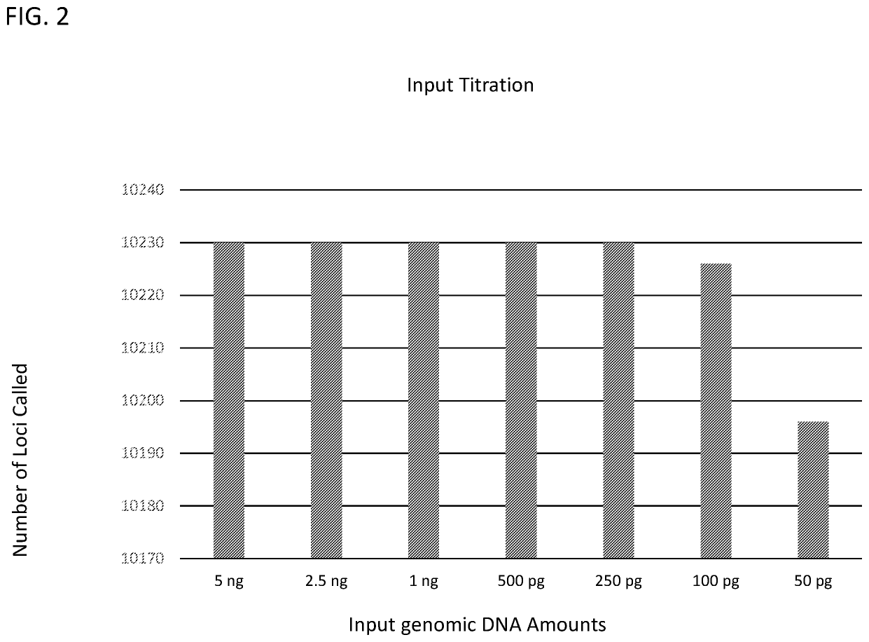

The denatured libraries were sequenced as per instructions on MiSeq FGx Sequencing System Reference Guide (document #VD2018006, the contents of which are hereby incorporated by reference in their entirety). As shown in FIG. 2, the number of loci detected were similar across a range of input genomic DNA titrations.

Results were analyzed using the Forenseq Universal Analysis Software 2.1 (Verogen, San Diego, CA) following the instructions outlined in Forenseq Universal Analysis Software 2.1, and provided in Reference Guide Document #VD2019002, the contents of which are hereby incorporated by reference in their entirety.

### Example 2: Generation of Sequence Libraries Using Degraded DNA

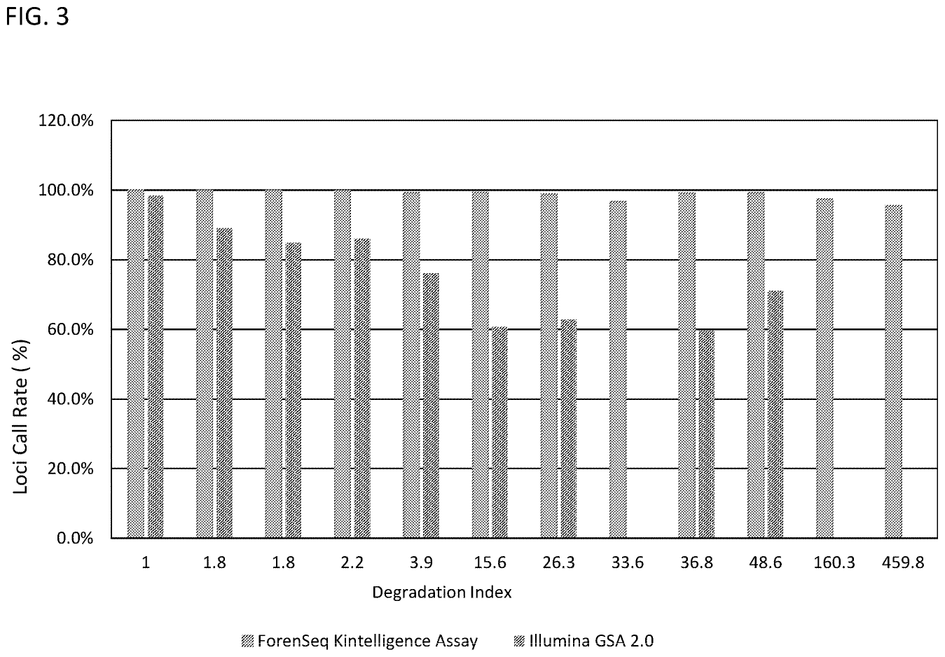

This Example describes the sequencing of DNA from low quantity and highly degraded samples. Degraded DNA A series of degraded blood DNA was obtained from Innogenomics (New Orleans, LA). The DNA samples were used to generate sequencing libraries as described in Example 1, with the exception that primer pairs for 10,327 loci were used in this example. The percentage of Loci detected (call rate) with degraded DNA using the assay described herein compared to Microarray (GSA) call rate is shown in FIG. 3. The degradation Index (DI) is shown on x-axis and the number of detected loci on Y-axis. These results show that even with highly degraded DNA with a DI of 158.3, the assay detected 9167 loci, which is sufficient to upload to the genealogy database to search for relatives. The alternative technologies such as Microarrays failed to detect any loci in samples with high degradation index.

### Example 3: Assessment of Activity of Inhibitors on Library Preparation

This Example describes assessment of the effect of PCR inhibitors on the preparation of libraries disclosed herein. DNA samples from crime scenes often contain co-purified impurities which inhibit PCR. PCR inhibition is the most common cause of PCR failure when adequate copies of DNA are present. Humic compounds, a series of substances produced during decay process have been considered as the materials contaminating DNA in soil, natural waters and recent sediments. Other common inhibitors include hematin (from blood), indigo (from blue jeans) and tannic acid.

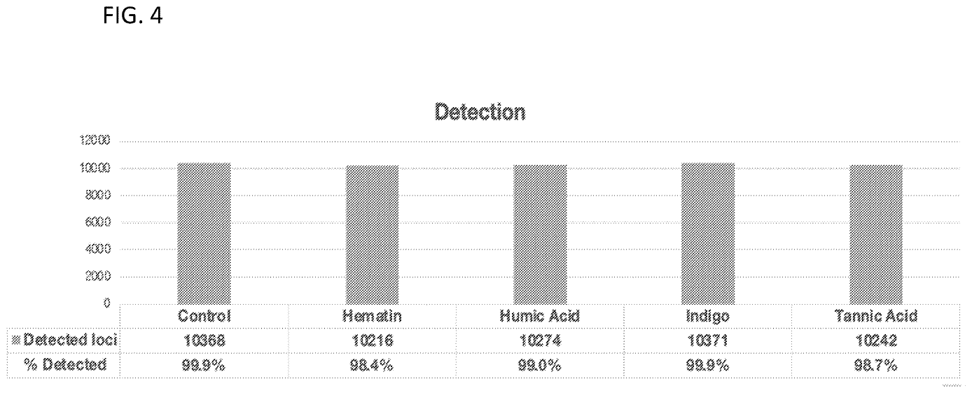

To assess the impact of inhibitors commonly found in forensic samples, library preparation was performed as described in Example 1, with the exceptions of 200 μM Hematin, 50 ng/μL Humic Acid, 133 μM Indigo, 16 μM Tannic Acid were spiked into the “Amplify and Tag targets” step above and primer pairs for 10380 loci were used. Results are shown in FIG. 4, with a PCR reaction without any inhibitors is labeled as Control.

### Example 4: Determination of Degree of Relationship

This Example describes exemplary results from samples prepared generally as described in Example 1 above.

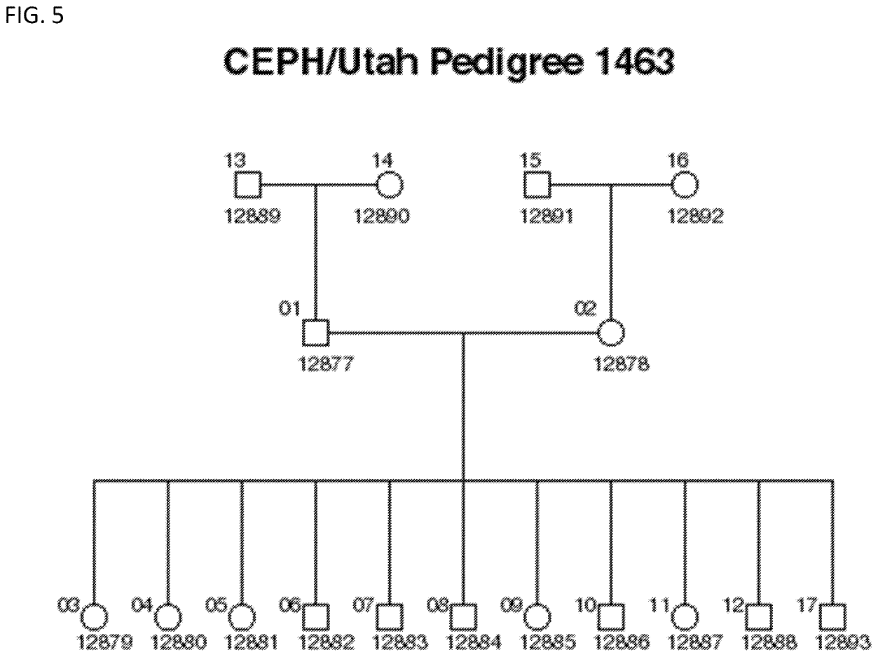

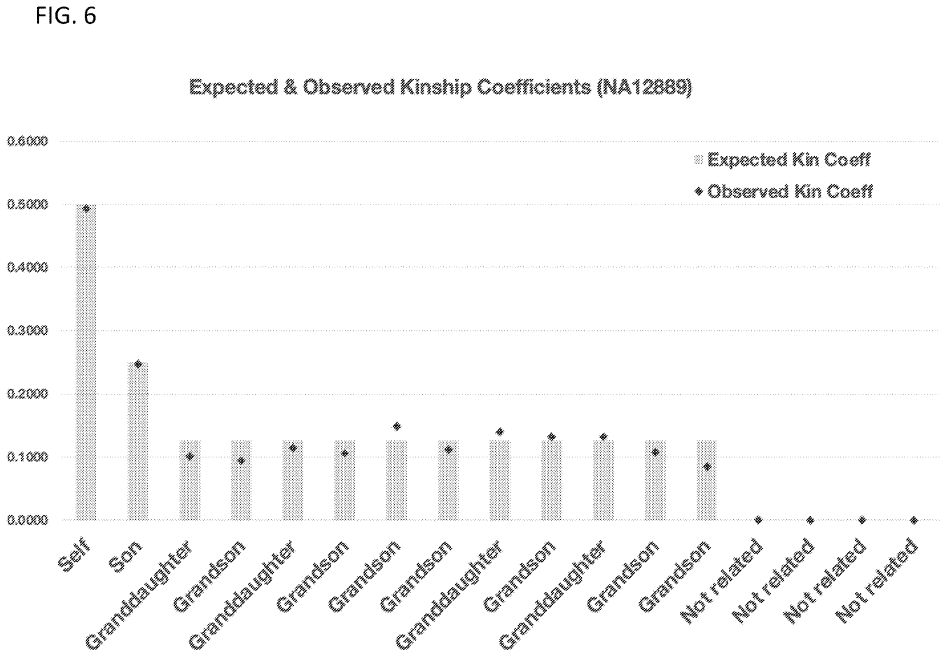

Illumina Global Screening Array (GSA) 2.0 were run with 200 ng each of 17 samples of Utah CEPH family 1463 DNA (Coriell Institute). The SNP calls were uploaded to the GEDmatch database (Verogen). An exemplary family tree is shown in FIG. 5. One of the samples, NA12889 (paternal grandfather) was run in the library preparation protocol as described in Example 1, run on ForenSeq UAS 2.1 module. The generated report was uploaded to the database and searched using the 1: many tool for searching relationships. The kinship coefficients from the algorithm in the database were compared to the expected kinship coefficients. The expected and observed kinship coefficients are shown in FIG. 6.

### Example 5: Kinship Coefficient Determination in Exemplary Case Study

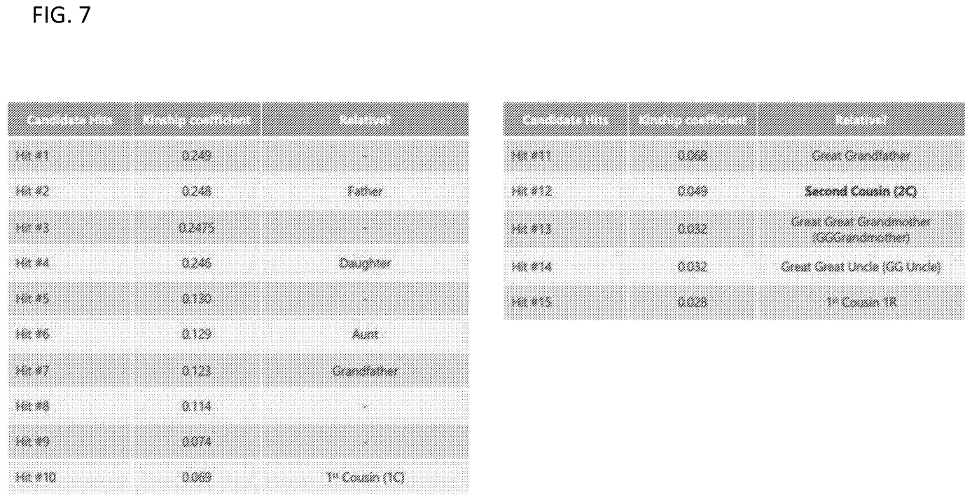

This Example describes the results of an exemplary case study using a sample SNP profile to determine kinship coefficient. The ability of the 1: many search algorithm to detect potential relatives was tested using 10 established pedigrees with 12-28 family members in the GEDmatch database. The sample SNP profile from the assay disclosed herein was considered to be of Mr. X=POI (Person of interest/unknown crime scene profile). Candidate hits, kinship coefficient and relative status are shown in FIG. 7.

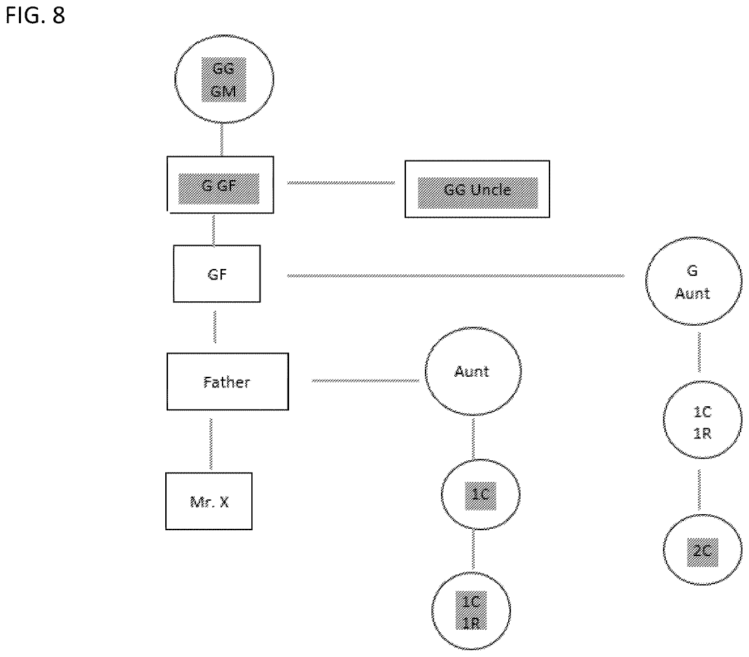

The results generated from the search algorithm were then used to generate the family tree for Mr. X as shown in FIG. 8. As shown in the family tree, Mr. X's first cousin (1C) and great grandfather (G GF) which are 3^{rd} degree relationships; were returned within the first 11 candidate hits. Mr. X's Great Great Grandmother (GG GM), Great Great uncle (GG uncle) and First cousin once removed (1C1R), which are 4^{th} degree relationships were returned within the first 15 candidate hits. Mr. X's second cousin (2C), a 5^{th} degree relationship was the 12^{th} hit.

### Example 6: Generation of Sequence Libraries and Determination of Sensitivity, Including Assessment by the Type of Loci

This Example involves a method of determining the sensitivity of the multiplex polymerase chain reaction described herein to generate libraries capable of being sequenced, and includes an assessment by the type of loci.

Sequence libraries (sequenced nucleic acid libraries), also referred to as DNA profiles, were generated in the same manner as described in Example 1, except that results were analyzed using the Forenseq Universal Analysis Software version 2.2.

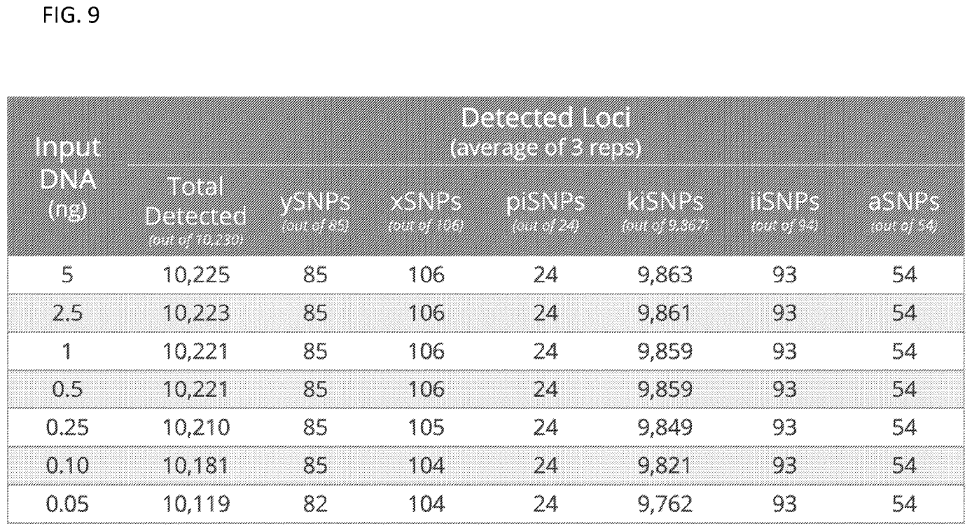

The results are shown in FIG. 9, which is a table summarizing the number of detected loci (as an average of three replicates) based on the amount of input DNA (ng) for each of the different types of loci, e.g., Y-chromosome SNPs (Y-SNPs), X-chromosome SNPs (X-SNPs), phenotype SNPs (piSNPs), kinship SNPs (kiSNPs), identity SNPs (iiSNPs), and biogeographical ancestry SNPs (aiSNPs), out of a total of 10,230 total loci being analyzed. Input titrations of genomic DNA tested included 5 ng, 2.5 ng, 1 ng, 0.5 ng (500 pg), 0.25 ng (250 pg), 0.10 ng (100 pg), and 0.05 ng (50 pg) of input genomic DNA. As shown in FIG. 9, the total detected SNPs, each of the amounts of input DNA ranging from 0.05 ng to 5 ng resulted in at least 98.9% (10,117) of the loci being detected, and the amounts of input DNA of 0.10 ng and greater resulted in at least 99.5% (10,179) of the loci being detected.

This data demonstrates that more than 10,000 loci can be detected at a high efficiency and a high sensitivity using different types of SNPs and using amounts of input DNA ranging from 0.05 ng (50 pg) to 5 ng.

### Example 7: Assessment of Activity of Inhibitors on Sequence Library Preparation, Including Assessment By the Type of Loci

This Example describes an assessment of the effect of certain inhibitors on the preparation of sequence libraries (sequenced nucleic acid libraries) also referred to as DNA profiles, disclosed herein, including by type of loci being detected and sequenced. DNA samples from crime scenes often contain co-purified impurities which inhibit amplification. Common inhibitors include Hematin, Humic Acid, and Indigo.

To assess the impact of inhibitors commonly found in forensic samples, library preparation was performed as described in Example 1, except that results were analyzed using the Forenseq Universal Analysis Software version 2.2, and an assessment of the impact of certain inhibitors on amplification was performed as described in Example 3, with the exception that the inhibitors tested were as follows: 200 μM Hematin, 100 μM Hematin, 50 ng/μL Humic Acid, 25 ng/μL Humic Acid, 16 μM Tannic Acid, 8 μM Tannic Acid, 133 μM Indigo, and 66.5 μM Indigo were included in the amplification step as described in Example 1, and primer pairs for 10230 loci were used. A positive control reaction without any inhibitor included was also performed. 1 ng of input DNA was used.

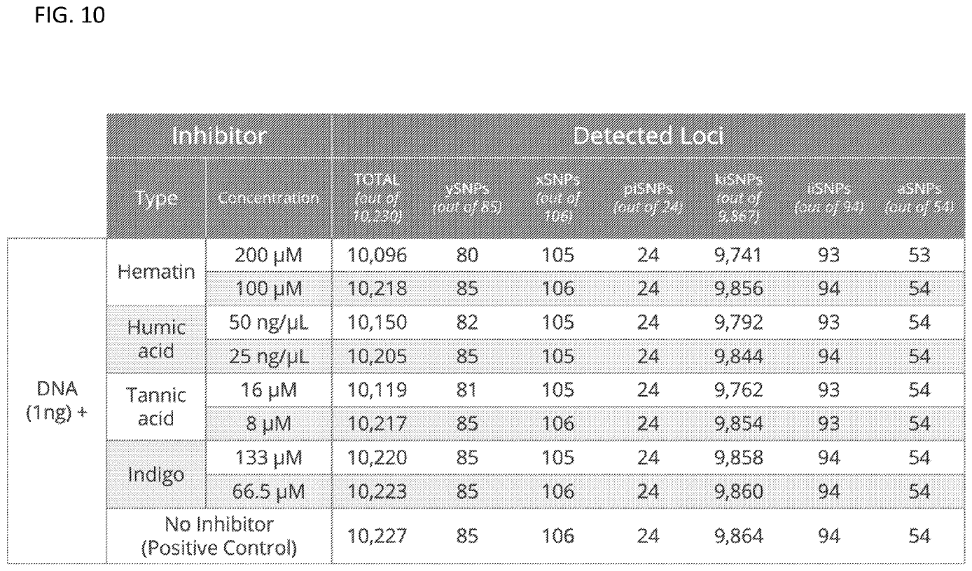

The results are shown in FIG. 10, which demonstrates that various SNPs including kiSNPs, Y-SNPs, X-SNPs, piSNPs, iiSNPs, and aiSNPs can be amplified and detected in combination with one another in accordance with the methods described herein with a high rate of efficiency and detection, as demonstrated by, e.g., all or nearly all of the SNPs of each type being detected even when in the presence of the inhibitor. For instance, the number of detected kiSNPs, Y-SNPs, X-SNPs, piSNPs, iiSNPs, and aiSNPs are each similar to the number detected in the positive control that lacked an inhibitor (FIG. 10). This data demonstrates that the presence of common inhibitors in samples does not have a detrimental impact on the ability to amplify more than 10,000 SNPs in PCR reactions using the methods described herein.

### Example 8: Assessment of Sequence Library Preparation Using DNA Samples Obtained Following a Mock Sexual Assault

This example describes the generation of sequence libraries (sequenced nucleic acid libraries) also referred to as DNA profiles, using DNA from mock sexual assault samples, in order to confirm whether sequence libraries, e.g., sequenced nucleic acid libraries, could be successfully generated using a low amount of input DNA, e.g., less than the recommended amount of 1 ng, such as 500 pg . . . .

Mock sexual assault DNA was obtained from samples collected at 9 hours and 22 hours after the occurrence of a mock sexual assault. DNA was isolated from the sperm fraction using a differential extraction method, with sperm fractions from both time points collected and saved for analysis. The amount of DNA from the sperm fraction that was available as input in the assay (for the generation of a sequence library) was only 500 pg, which is half of the recommended amount of 1 ng.

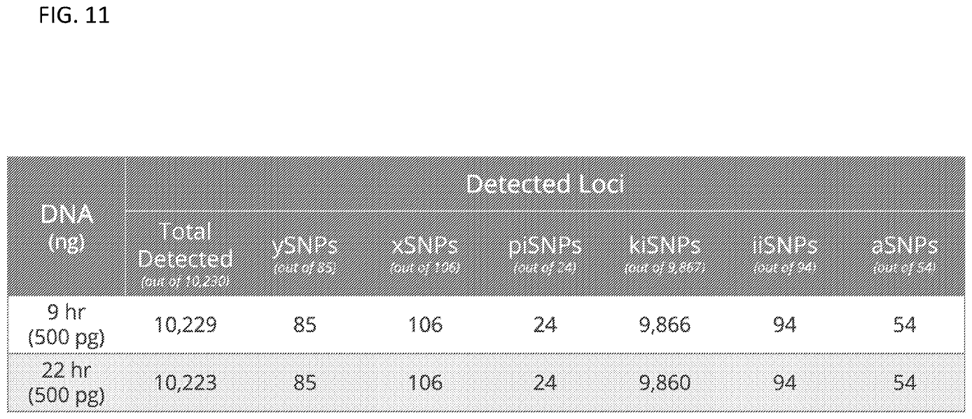

The DNA samples were used to generate sequence libraries (sequenced nucleic acid libraries) as described in Example 1, except that results were analyzed using the Forenseq Universal Analysis Software version 2.2. The percentage of loci detected (call rate) as well as the number of each type of SNP present in the assay are shown in FIG. 11. The results demonstrate that even with only 500 pg of input DNA, the majority of SNPs are detected, with 99.99% of all SNPs (10,229 out of 10,230 SNPs) being detected at the 9 hour time point, and 99.93% of all SNPs (10,223 out of 10,230 SNPs) being detected at the 22 hour time point. Specifically, all aiSNPs, iiSNPs, piSNPs, X-SNPs, and Y-SNPs were detected at both the 9 hour and 22 hour time points after the occurrence of the mock sexual assault. Only one kiSNP out of 9,867 was not detected at the 9 hour time point, and only seven kiSNPs out of 9,867 were not detected at the 22 hour time point. The number of loci detected is sufficient to upload to the genealogy database to search for relatives.

This data demonstrates that the methods described herein can be used to detect more than 10,000 SNPs, including various kiSNPs, Y-SNPs, X-SNPs, piSNPs, iiSNPs, and aiSNPs, and create sequence libraries, using only 500 pg of DNA at 9 hours and 22 hours after occurrence of a mock sexual assault, with more than 99.9% of all SNPs detected. Accordingly, the methods described herein are suitable for use in creating sequence libraries with less than recommended amounts of DNA, e.g., 500 pg, following criminal incidents, including sexual assaults, or from victims of disaster or conflicts, or from samples left behind by missing persons.

### Example 9: Assessment of PCIA Carry-Over on Generation of Sequence Libraries From Saliva Samples

This Example describes the sequencing of nucleic acid libraries (e.g., to generate DNA profiles) from DNA derived from saliva samples that was extracted using organic extraction with the phenol-chloroform-isoamyl alcohol (PCIA) extraction method.

Saliva DNA was obtained from saliva samples where increasing amounts of the extraction reagent PCIA (e.g., no PCIA, light PCIA, moderate PCIA, and heavy PCIA) were intentionally left with the extracted DNA as carry-over, which simulates less than perfect extraction. PCIA, including its ingredient phenol, is a known inhibitor of PCR amplification.

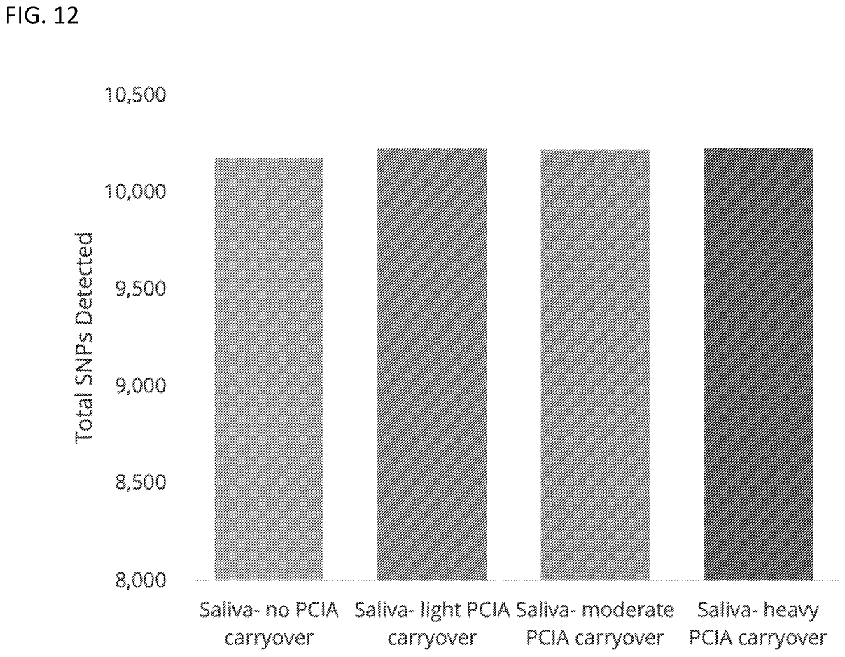

The DNA samples having no PCIA, light PCIA, moderate PCIA, or heavy PCIA were used to generate sequence libraries (sequenced nucleic acid libraries) as described in Example 1, except that results were analyzed using the Forenseq Universal Analysis Software version 2.2. The total number of SNPs detected for each sample was determined and is shown in FIG. 12. The results show that PCIA carry-over, even at high levels with heavy PCIA carry-over, does not affect the ability for the assay to detect SNPs since more than 10,170 SNPs were detected in each of the samples.

### Example 10: Assessment of Generation of Sequence Libraries from Blood Samples on Various Substrates and Impact of Heme

This example describes the sequencing of nucleic acid libraries (e.g., to generate DNA profiles) on DNA derived from blood samples deposited in different substrates typically found at crime scenes, including rust and denim, as well as a blood sample on a swab where only 420 pg of DNA was available, and blood samples extracted using CheleX™ where increasing levels of heme was carried over with the DNA. Heme is a known inhibitor of PCR amplification. Denim contains indigo dye, which is a known inhibitor of PCR amplification.

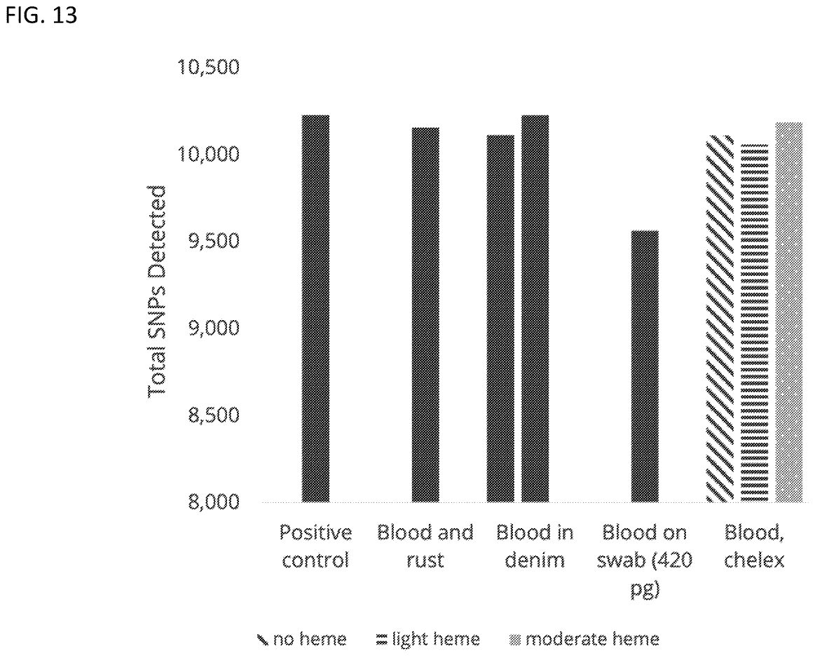

Each of the DNA samples was used to generate a sequence library (sequenced nucleic acid library) as described in Example 1, except that results were analyzed using the Forenseq Universal Analysis Software version 2.2, including a sample containing blood and rust, two blood samples in denim, a 420 pg blood sample on a swab, and blood samples with light or moderate amounts of heme carry-over or no heme as a control, as well as a positive control blood sample. The total number of SNPs detected for each sample and a reference control was determined and are shown in FIG. 13. The results show that the blood samples deposited in different substrates still allowed for the detection of 10,114 or more SNPs out of 10,230 total SNPs. The blood sample with only 420 pg yielded the detection of 9,563 SNPs, and the samples with heme yielded more than 10,000 SNPs detected, and the number of SNPs detected was not affected by the amount of heme present in the sample. This demonstrates that DNA extracted from blood samples deposited on various substrates commonly found at crime scenes can be used in accordance with the methods provided herein to detect more than 10,000 SNPs for forensic applications.

### Example 11: Kinship Analyses Using Related Samples, Related Antemortem Samples, Unrelated Postmortem Samples, and Related Mock Postmortem Samples

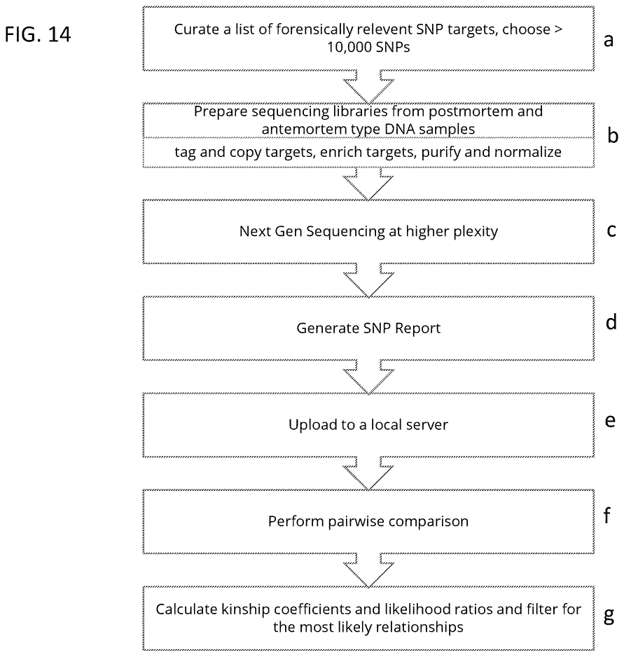

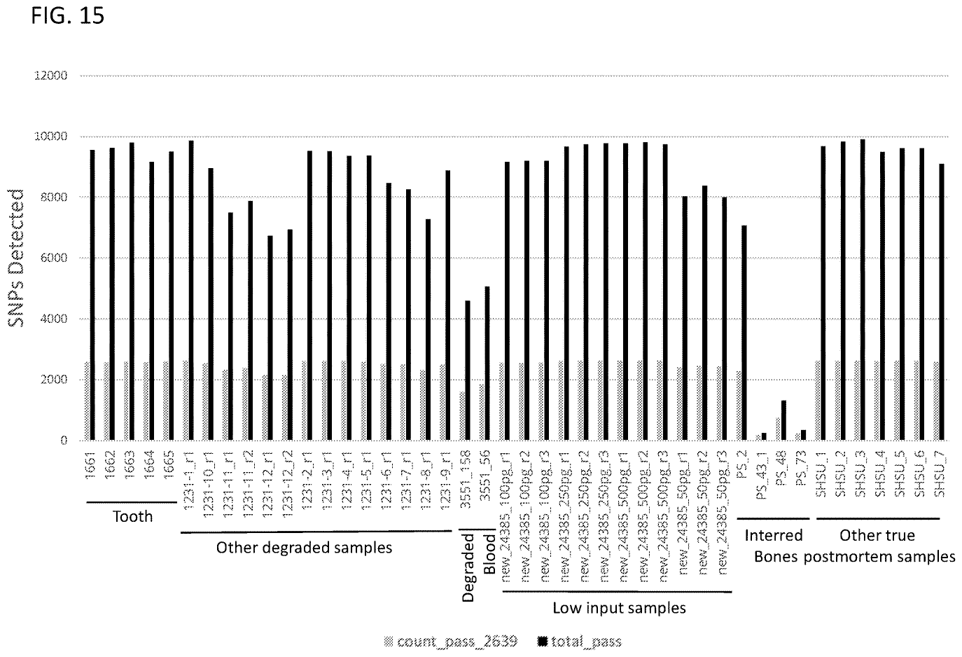

This example describes performing kinship analysis as described herein to identify up to third degree relationships in four different sets of samples, including non-degraded, highly degraded, and low input samples. Specifically, a goal of this example was to determine up to third degree familiar relationships from degraded samples that are sequenced in high plexity while still allowing for enough SNPs to accurately predict such family relationships, with potential matches being in a local, private database (rather than a publicly accessible database). A schematic overview of the methodology involved is depicted in FIG. 14, which includes the steps of (a) curating a list of forensically relevant SNP targets, and choosing >10,000 SNPs, such as 10,230 SNPs; (b) preparing sequencing libraries from postmortem and antemortem type DNA samples, by tagging and copying the targets, enriching the targets, purifying the targets, and normalizing the target amounts; (c) performing next generation sequencing at a higher plexity, e.g., 12plex or higher; (d) generating a SNP report (also referred to as a DNA profile); (e) uploading the SNP report to a local server; (f) performing pairwise comparison; and (g) calculating kinship coefficients and likelihood ratios, and filtering for the most likely familiar relationships. In some embodiments, the curating in step (a) was performed in a previous workflow and the same selected SNP targets, e.g., a specific set of 10,230 SNP targets, are utilized in the present workflow.

A set of 10,230 SNP targets was selected for detecting in each of the four sets of samples. Four different sets of samples were sequenced to generate a sequence library as described in Example 1. These four different sets of samples include: (1) a set of related antemortem samples from CEPH/Utah that include up to second degree relationships verified at Coriell (herein referred to as “related antemortem CEPH/Utah samples”); (2) a set of related antemortem samples from a private family that includes up to fifth degree relationships (herein referred to as “related antemortem private family samples”); (3) a set of unrelated postmortem samples that includes bones (cremated, embalmed, burned, and interred), dental remains/teeth, and degraded blood of varying degradation index (DI) levels (herein referred to as “true postmortem samples”); and (4) a set of related mock postmortem samples that include the same samples from set (2) but includes DNA that was either (a) artificially degraded by boiling the DNA for 24 hours (DI range of 2.1-20) or (b) sequenced at a low input of DNA (50 pg) (herein referred to as “related mock postmortem samples”).

The true postmortem samples were run at 12-plex using the MiSeq FGx Sequencing System, and the results are shown in FIG. 15, which shows the number of SNPs detected for individual samples within this set of true postmortem samples. This includes true postmortem samples labeled as “tooth,” “degraded blood,” “interred bones,” “low input samples,” “other degraded samples,” and “other true postmortem samples.” As shown in FIG. 15, for the true postmortem samples, 3 of the 4 interred bone samples had the lowest number of SNPs detected (or called), which ranged from 248 to 1319 SNPs, and the degraded blood samples with the highest DI (DI 158 and DI 56) had the next lowest number of SNPs detected at 4,603 and 5,069 SNPs detected, respectively. All of the remaining true postmortem samples had a minimum of 6,737 SNPs detected and a maximum of 9,903 SNPs detected (FIG. 15). The “total pass” counts reflect the total number of detected SNPs for each sample out of the full set of 10,230 SNPs, and the “count pass” counts reflect the total number of detected SNPs from among a subset of 2,639 of the SNPs that are consistently called across samples. As shown in FIG. 15, there is a core set of SNPs that are consistently called, i.e., detected, across samples, since there is less variation in the number of detected SNPs from among the subset of 2,639 SNPs (the “count pass” SNPs) than the total number of called SNPs overall.

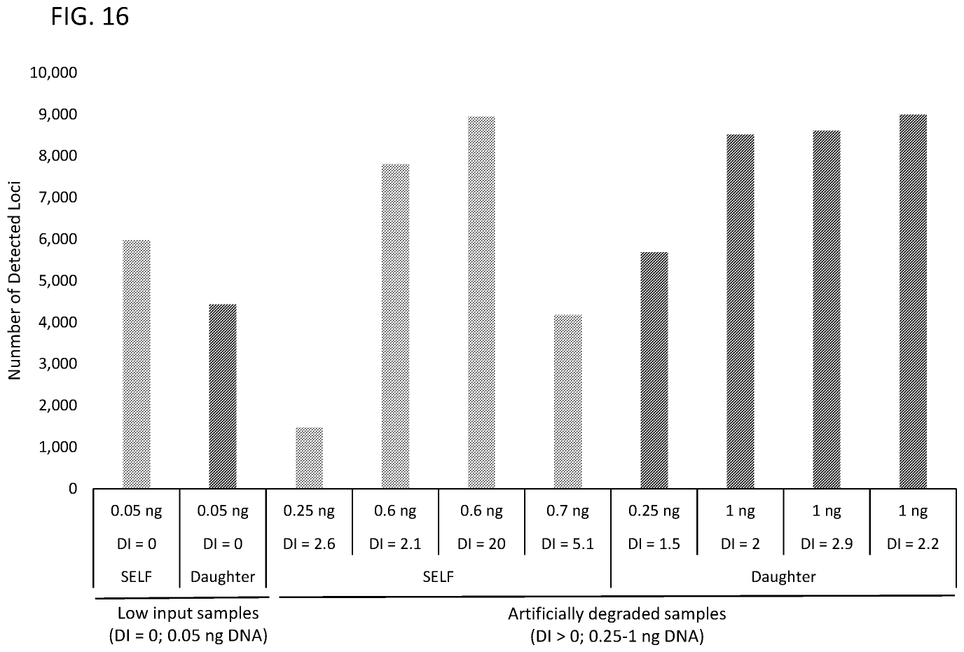

The mock postmortem samples were also run at 12-plex using the MiSeq FGx Sequencing System, and results are shown in FIG. 16. As shown in FIG. 16, the related mock postmortem samples that were artificially degraded by boiling, i.e., the samples having a DI above 0, had a range in the number of SNPs detected that was between 1,470 and 8,999, with an average of 6,462 SNPs being detected for samples from a related parent and daughter. The low input DNA samples had a DI of 0 and an input of 0.05 ng of DNA (FIG. 16).

The related antemortem CEPH/Utah samples were run at 12-plex, 16-plex, 24-plex, and 32plex using the MiSeq FGx Sequencing System, to determine the highest plexity that would yield a high enough number of detected SNPs (i.e., SNP call rate) for the kinship analysis. The 24-plex sequencing run resulted in detecting 9,691 SNPs on average, which ranged from 8,297 SNPs detected up to 9,982 SNPs detected, depending on the sample; and the 32-plex sequencing run resulted in detecting 9,048 SNPs on average, which ranged from 6,894 SNPs detected up to 9,827 SNPs detected (data not shown). This demonstrated that a 30-plex run would allow for sufficiently high throughput of SNPs detected without significantly compromising the number of SNPs detected and the confidence of the kinship analysis.

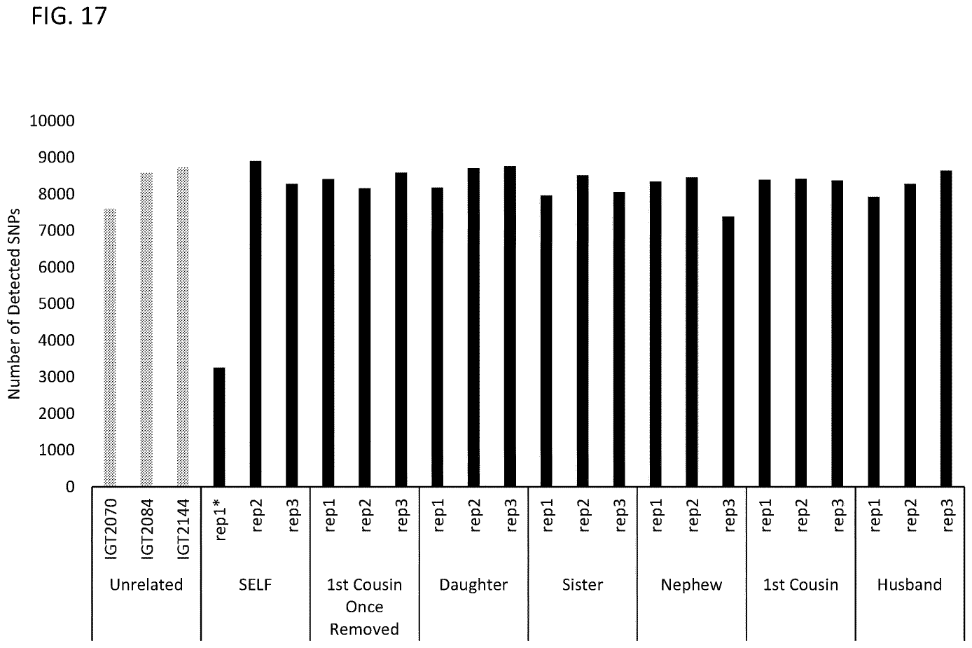

The related antemortem private family samples along with three unrelated samples were sequenced using a 30-plex sequencing run using the MiSeq FGx Sequencing System, and results are shown in FIG. 17. As shown in FIG. 17, over 7,000 SNPs were detected in each of the samples except for one replicate of the “self” sample (labeled as “rep1*”), which was likely due to an error in the library preparation for that particular sample. The samples of which over 7,000 SNPs were detected includes the “self” sample, and several relatives of the “self” individual, including samples from a first cousin once removed, a daughter, a sister, a nephew, a first cousin, and a husband.

A kinship analysis was then performed using the DNA profile that was generated following the sequencing runs. The related antemortem private family samples (sequenced at 30-plex) and the mock postmortem samples (sequenced at 12-plex), which were derived from the same original related samples but with the related mock postmortem samples having been artificially degraded or used at a low input, were compared. Using a minimum kinship coefficient value of 0.031, all expected relationships up to a third degree (e.g., first cousins) were matched, and no false matches (e.g., no false positives) were obtained, thereby resulting in 100% specificity and 100% sensitivity (data not shown). Some, but not all, of the expected fourth degree matches (e.g., first cousin once removed) were obtained (data not shown). These data confirm that this method can accurately identify up to third degree relationships while excluding all unrelated relationships, even with highly degraded and low input samples, such as those that may be available in situations of missing persons and disaster/conflict victims.

Next, to evaluate the most suitable kinship coefficient threshold, the related mock postmortem samples (sequenced at 12-plex) were compared to the GEDMatch database, with the assumption that the related individuals in represented by the related mock postmortem samples do not have any close relatives in the GEDMatch database. It was determined that to achieve 100% specificity (i.e., no false positives), the kinship coefficient would need to be 0.062. Applying this kinship coefficient value threshold (0.062) to the related mock postmortem samples (sequenced at 12-plex) as compared to the related antemortem private family samples (sequenced at 30-plex), it was determined that this would decrease sensitivity by excluding some of the known third degree relationships.

### Example 12: Assessing a Minimum Number of Snps to Accurately Determine Kinship

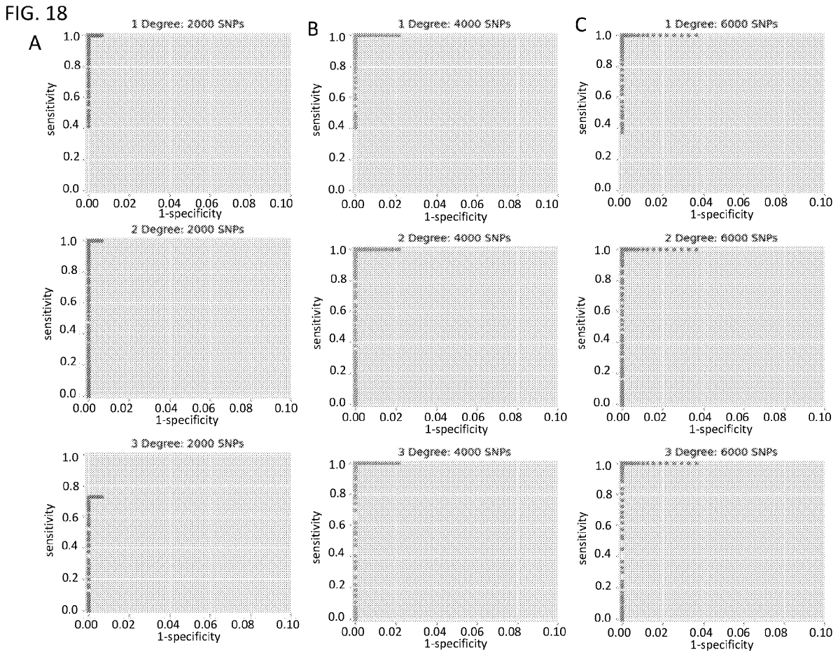

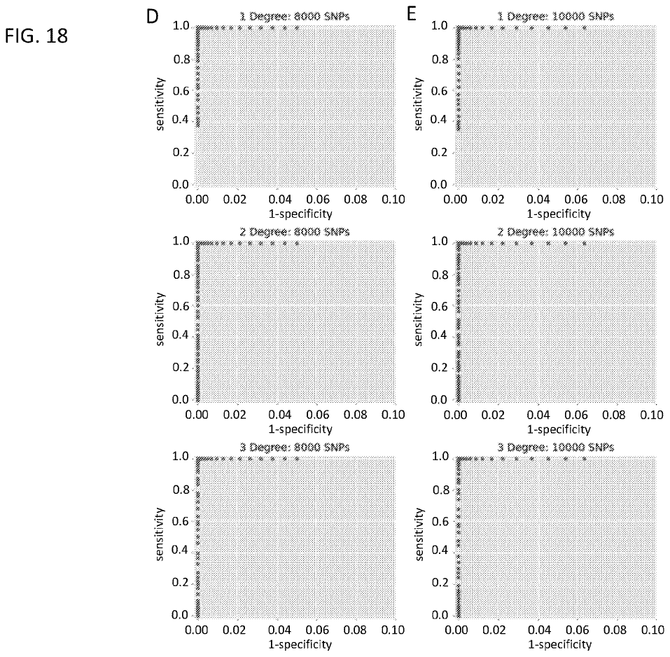

To identify the minimum number of detected SNPs (i.e., the SNP call rate) to accurately determine kinship, known relationships within the GEDMatch database that simulate a range of detected SNPs (i.e., called SNPs) were compared. This included testing call rates of 2,000 SNPs, 4,000 SNPs, 6,000 SNPs, 8,000 SNPs, and 10,000 SNPs for sensitivity and specificity at identifying first degree, second degree, and third degree familial relationships. The results are shown in FIG. 18A-E, which depicts receiver operating characteristic (ROC) curves of the results for 2,000 SNPs (FIG. 18A), 4,000 SNPs (FIG. 18B), 6,000 SNPs (FIG. 18C), 8,000 SNPs (FIG. 18D), and 10,000 SNPs (FIG. 18E). The lowest SNP call rate (n=2,000) resulted in reduced specificity at first, second, and third degree relationships (FIG. 18A), which suggested that a SNP call rate of 2,000 SNPs detected was an absolute floor in the SNP call rate for accurately identifying relationships.

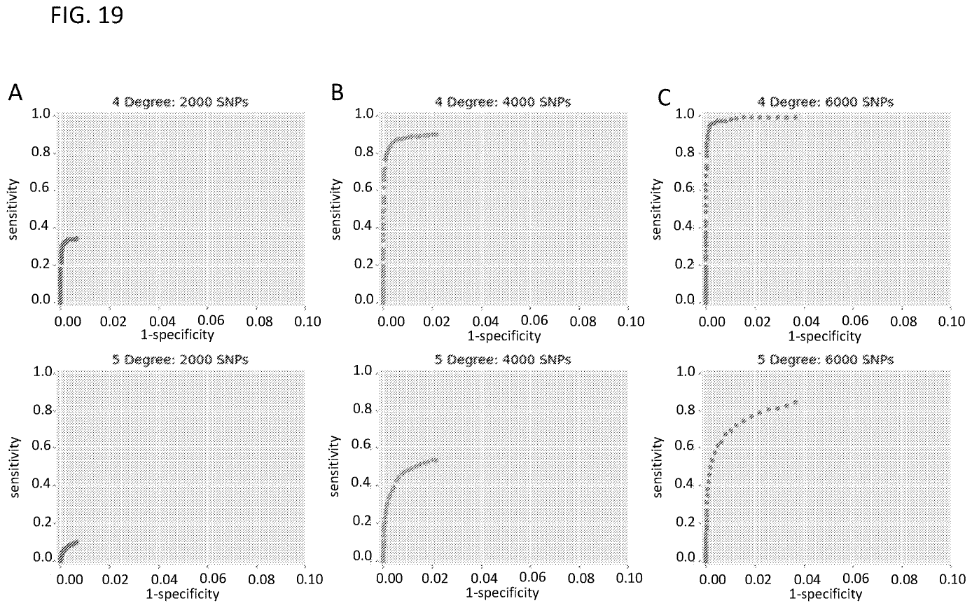

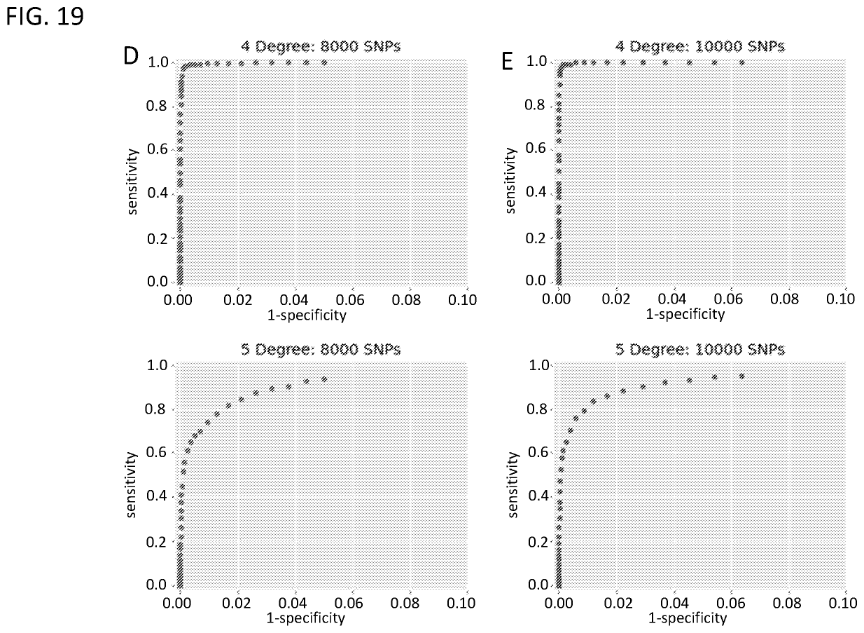

The ability to sequence non-degraded samples at 3-plex opens the possibility of identifying fourth and fifth degree relationships. A similar analysis using the GEDMatch database was performed, but for identifying fourth and fifth degree relationships. This included testing call rates of 2,000 SNPs, 4,000 SNPs, 6,000 SNPs, 8,000 SNPs, and 10,000 SNPs for sensitivity and specificity at identifying fourth and fifth degree familial relationships. The results are shown in FIG. 19A-E, which depicts ROC curves of the results for 2,000 SNPs (FIG. 19A), 4,000 SNPs (FIG. 19B), 6,000 SNPs (FIG. 19C), 8,000 SNPs (FIG. 19D), and 10,000 SNPs (FIG. 19E). As shown in FIG. 19A-E, a higher minimum number of called SNPs (~6,000) is required to accurately identify true fourth and fifth degree relationships.

### Example 13: High Plexity Snp Sequencing for Kinship Analyses Using Related Samples, Mock Antemortem Samples, and Mock Postmortem Samples

This example describes performing kinship analysis as described herein to identify the degree of relationship in different sets of samples, including non-degraded, highly degraded, and low input postmortem (PM) and antemortem (AM) samples. Specifically, a goal of this example was to determine up to third degree familiar relationships from degraded samples that are sequenced in high plexity while still allowing for enough SNPs to accurately predict such family relationships, with potential matches being in a local, private database (rather than a publicly accessible database). A schematic overview of the methodology involved is depicted in FIG. 14, which includes the steps of (a) curating a list of forensically relevant SNP targets, and choosing >10,000 SNPs, such as 10,230 SNPs; (b) preparing sequencing libraries from postmortem and antemortem type DNA samples, by tagging and copying the targets, enriching the targets, purifying the targets, and normalizing the target amounts; (c) performing next generation sequencing at a higher plexity, e.g., 12plex or higher; (d) generating a SNP report (also referred to as a DNA profile); (e) uploading the SNP report to a local server; (f) performing pairwise comparison; and (g) calculating kinship coefficients and likelihood ratios, and filtering for the most likely familiar relationships. In some embodiments, the curating in step (a) was performed in a previous workflow and the same selected SNP targets, e.g., a specific set of 10,230 SNP targets, are utilized in the present workflow. In some embodiments, a windowed kinship alogirthm is used.

### A. Methodologies

A set of 10,230 SNP targets was selected for detecting in each set of samples, including mock antemortem samples and mock postmortem samples.

Libraries are generated as described in Example 1 with 1 ng of NA24385 DNA as a positive control in these studies and the results automatically analyzed in the ForenSeq Universal Software version 2.3 (UAS) with expected quality control metrics.

Commercially obtained intact DNA, used as mock antemortem (AM) samples, were purchased for these studies, including eighty-one DNA samples from 1000 Genomes Project, CEPH collection, or Personal Genome Project DNA samples from Coriell Institute for Medical Research (Camden NJ, USA), and four DNA samples extracted from whole blood from Innogenomics Inc. (New Orleans, LA, USA). The low input DNA samples included the sample NA24385 at amounts of 0.05 ng, 0.1 ng, 0.25 ng, and 0.5 ng.

Mock postmortem (PM) sample DNA extracts consisted of five contemporary tooth (CT) samples designated CT1, CT2, CT3, CT4, and CT5, seven contemporary bone samples, and one DNA extract from an ancient bone of Eastern European origin. The DNA from the seven contemporary bone (CB) samples was extracted using either the PrepFiler™ forensic DNA extraction kit (Thermo Fisher, Waltham, MA, USA) for samples CB 1, CB 3, CB 4, CB 6, and CB 7, or demineralization protocol for bone samples CB 2 and CB 5. The degradation index and DNA concentration of the CB bone DNA samples was determined using Quantifiler™ Trio DNA Quantification Kit (Thermo Fisher, Waltham, MA, USA). The DIs of the CB samples were 13.6, 4.3, 5.6, 1.1, 1.8, 2.5, and 6.5 for CB1, CB2, CB3, CB4, CB5, CB6, and CB7, respectively.

Additionally, intentionally degraded DNA was used as either mock AM or mock PM samples. Two series of degraded DNA were purchased from Innogenomics Inc. (New Orleans, LA, USA): DNA was extracted from whole blood using organic methods from two different male donors and sheared by sonication at 50 °C for times ranging from 0 to 16 hours (Samples 1231 and 3551). The amount of human DNA and the degradation state was also determined. The 1231 samples had DIs of 26.3, 33.6, 48.6, 160.3, and 459.8 for 1231 samples designated as 7, 8, 10, 11, and 12, respectively. The 3551 samples had DIs of 56 and 158.3 for 3551 samples designated as 56 and 158, respectively.

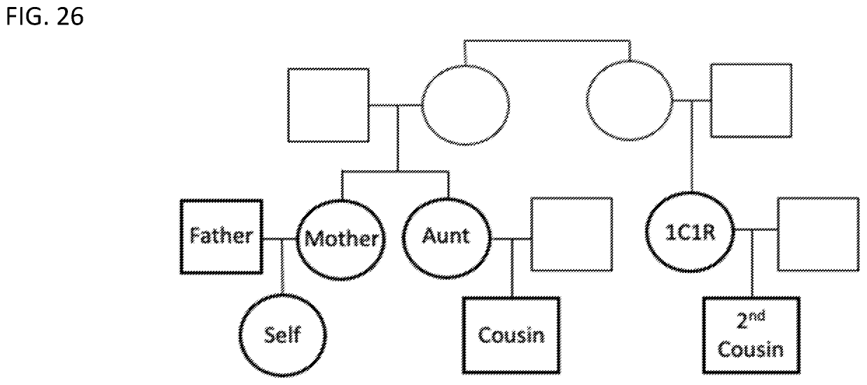

Buccal samples were collected from volunteers from a family with a known pedigree (RF004, RF016-021), herein referred to as the Related Family (RF), which has a pedigree as depicted in FIG. 26. DNA was extracted and purified from buccal swabs. Two of the DNA samples from the Related Family (RF004 and RF016) were artificially degraded using high temperature treatment as follows: five replicates of purified buccal DNA from each individual were subjected to 21 cycles of heating and chilling at 98 °C for 1 hour followed by 4 °C for 10 minutes. DNA grade water was added to the subsequently dried DNA to bring the DNA into solution. Degradation indices and DNA concentration was determined for all Related Family DNA samples. Degradation indexes varied for replicates with values of 1, 2.1, 2.6, 5.1 and 20 for sample RF004 and 1, 1.5, 2.0, 2.2, and 2.9 for sample RF016.

DNA sequence libraries were prepared using the ForenSeq Kintelligence Kit (Verogen, San Diego, CA, USA) following the manufacturer's instructions, and libraries were quantified using the QuantiFluor ONE dsDNA system (Promega, Madison, WI, USA). Unique dual indexed adapters (UDIs) were utilized when sequencing the libraries using higher plexity. Prior to library preparation, intact DNA samples were quantified for input into library preparation. Mock PM DNA samples were quantified utilizing qPCR methods. Unless otherwise noted, the DNA was diluted to 40 pg/pL for 1 ng total DNA added to the library preparation reaction. The Positive Control DNA NA24385 was serially diluted to 20, 10, 4, and 2 pg/uL for total DNA inputs of 500, 250, 100, and 50 pg to mimic low input PM samples. The purchased, artificially degraded samples had DNA concentrations sufficient to add 1 ng of DNA to the library preparation reactions. Not all of the degraded Related Family samples had sufficient DNA concentration for input of 1 ng DNA into the library preparation reactions. For sample RF004, degraded replicates with DI of 2.1, 20, 5.1, and 2.6, 600 pg, 600 pg, 700 pg and 250 pg was added to the library preparation reactions, respectively. For sample RF016, degraded replicates with DI of 2.0. 2.2 and 2.9 had sufficient DNA concentrations to add 1 ng to the library preparation reactions. To mimic low input samples, 50 pg of RF004 with DI of 1 and 50 and 250 pg of RF016 with DI of 1 and 1.5, respectively, was added to the library preparation reactions. The ancient bone DNA concentration was estimated to be 390 pg based on the mtDNA quantification of ~1400 mtDNA copies/μL. Each set of library preparations included one positive amplification control of 1 ng NA24385 DNA, and one negative template control (NTC).

After amplification of the targets and purification, libraries were normalized to 0.75 ng/μL. If library yields were lower than 0.75 ng/L, the library was pooled neat without dilution. Mock AM libraries generated from commercially obtained intact DNAs showed library yields >0.75 ng/μL with one exception at 0.67 ng/μL. Some libraries generated from mock PM, low DNA input, and commercially degraded DNA samples also had yields >0.75 ng/μL. Libraries were pooled at varying plexities by pipetting 8 μL of each normalized or neat library into a 1.7 ml microcentrifuge tube. Libraries generated from mock AM samples were pooled at sample plexities of 3, 12, 16, 24, 30, or 32 total libraries for denaturation and sequencing. Libraries generated from mock PM DNA samples were pooled at sample plexities of 3 or 12 total libraries for denaturation and sequencing. Pooled libraries were denatured with freshly diluted NaOH (HP3) by incubation at room temperature for 5 minutes and then followed by a dilution with HT1. Human sequencing control (HSC), a library consisting of 33 STRs that serves as a positive sequencing control, was also similarly denatured and diluted with HT1. The denatured library pools combined with denatured HSC were then sequenced on the MiSeq FGx instrument with the MiSeq FGx Reagent Kit following manufacturer's recommendations. Where possible, the sequencing runs were created in the ForenSeq Universal Analysis Software v2.2. Sequencing utilized 151 cycle paired-end reads for all libraries. The sequencing runs include two eight cycle indexing reads required to demultiplex the libraries utilizing the indices present in the UDI adapters.

To generate sequencing results for mock AM libraries with very high reads per sample for simulation studies, 30 libraries from mock AM samples were sequenced on the NextSeq 500 instrument using NextSeq 500/550 High Output Kit v2.5 (300 Cycles) kit (Illumina, San Diego, CA, USA) following manufacturer's recommendations (refs for NextSeq and Denaturation/pooling guide).

The sequencing data was then analyzed using secondary and tertiary data analysis as follows. Metrics are set with the UAS for the MiSeq FGx™ run quality. These metrics include cluster density, clusters passing filter, phasing, pre-phasing, and Q-score thresholds. Cluster density is the number of clusters (K) per square millimeter for the run and the metric is set to 400-1650 K/mm² for optimal sequencing results. The clusters passing filter metric measures quality of base calls via the percentage of clusters passing the Illumina chastity filter (ref) and the metric was set to ≥80%. When this metric fails, the number of usable reads is impacted but not the quality of those passing reads. The phasing metric represents the percentage of DNA strands in a cluster that fall behind the current cycle within a read and values of ≤0.25% are passing. Alternatively, pre-phasing represents molecules in a cluster that run ahead of the current cycle within a read and values of ≤0.15% are passing. If phasing or prephasing are out of specification, sequencing errors can be present at higher percentages. It is important to determine if the HSC passes its metrics before using the data from the run.

All library samples prepared with the six UDI adapters supplied in the ForenSeq Kintelligence Kit sequenced on the MiSeq FGx were analyzed with the ForenSeq Universal Analysis Software (UAS) v2.3 (Verogen, San Diego, CA) for allele and genotype calling as previously described (Jager et al., Forensic Sci. Int. Genet., 2017, 28:52-70, the content of which is hereby incorporated by reference in its entirety). For library samples prepared with additional UDI adapters for higher plexity, the sequencing runs were analyzed using the same bioinformatic pipeline that is utilized in the UAS but on a separate server through command line tools. This pipeline (run either within the UAS or through command line tools) has the same basic algorithm for SNP genotype calling present in the UAS v1.3 used for ForenSeq DNA Signature analysis (Jager ref) (Verogen, San Diego, CA, USA). First, samples are demultiplexed based on the supplied index sequences found on the UDI adapters by demultiplexing binary base call (BCL) files and generating FASTQ files. Reads 1 and 2 are aligned to the primer sequences using the Smith-Waterman-Gotoh algorithm (Gotoh, O., J. Mol. Biol., 1982, 162:705-708). Reads aligned to specific primer pairs are assigned to the loci corresponding to those pairs. Alignments were then written in the BAM format. At the position of each SNP, matches to the reference base call and matches to the alternate base call were counted, filtering on a minimum base quality of 30. Number of reads were then summed for each type of call (reference or alternate) at each locus, requiring a minimum coverage of greater than 10 reads. SNP genotypes were then determined by filtering on the analytical (AT) and interpretation (IT) thresholds, which were both set to 3%. AT and IT thresholds were determined by multiplying 3% by the sum of read counts at that locus. When low coverage occurred, a minimum of 650 reads was used to calculate the threshold values. The resulting AT and IT values were then compared to the total read counts for the reference and alternate alleles for each locus. Genotypes were determined for each locus if the call passed both AT and IT thresholds. Genotyped results were then written in the variant call format (VCF).

In some studies, GEDMatch data simulations were performed for whole genome kinship algorithm testing. For these studies, GEDMatch database profiles were downloaded and analyzed as described in Snedecor et al., Forensic Sci. Int. Genet., 2022, 61, 102769, doi: 10.1016/j.fsigen.2022.102769, the content of which is hereby incorporated by reference in its entirety. A set of 1000 anonymized samples, termed “query samples,” were randomly selected from GEDMatch. These samples were then queried for relatives in the GEDMatch database and any hits, termed “target samples,” were selected based on shared centiMorgans (CM) values calculated by the GEDMatch one-to-many tool. This search resulted in 2954 target samples along with the matched query sample. These results included query-target pairs that had zero shared cMs, representing true unrelated pairs. Therefore, the number of target samples is larger than the number of query samples due to inclusion of both related and unrelated target samples. Relationship degree was determined by comparing the resulting shared cM values generated from the one-to-many tool to the expected range of shared cM per degree provided by DNA Painter, accessible at: https://dnapainter.com/tools/sharedcmv4. The loci typed for the samples included in the query and target set were first filtered for the 10,230 SNPs in the panel, then randomly filtered to 80%, 60%, 40%, and 20% call rates, resulting in 8000, 6000, 4000, and 2000 loci, respectively, that were called in each query-target pair. The whole genome kinship coefficient was calculated for each query-target sample pair for each level of reduced locus call rate using the kinship algorithm. Pairs with a whole genome kinship coefficient greater than 0.031 were considered related; pairs with a whole genome kinship coefficient of less than or equal to 0.031 were considered unrelated. Sensitivity and specificity were calculated by comparing these results to the one-to-many tool query results. In other words, the one-to-many query results were considered the truth set and the results generated by the kinship algorithm were considered the test set.

Likeihood ratios (LRs) and kinship values were then calculated as follows. The LRs were calculated using the algorithms pedprobr (Brustad et al., Int. J. Legal Med., 2021, 135:117-129, the content of which is hereby incorporated by reference in its entirety) and dvir (Vigeland et al., Scientific Reports, 2021, 11:13661, the content of which is hereby incorporated by reference in its entirety). An average of the population frequencies from the Genome Aggregation Database (gnoMAD) (Karczewski et al., Nature, 2020, 581:434-443, the content of which is hereby incorporated by reference in its entirety) v3.0 was used to in the LR calculations. No mutation model was used, and theta was set to 0 as the SNPs chosen for the analysis have low linkage disequilibrium (Karczewski et al., supra). The LR was calculated as follows

$$L R = \frac{P ( D \vert {H}_{r} )}{P ( D \vert {H}_{u} )}$$

where D represents the genotypes, Hᵣ represents the hypothesis that the individuals are related, and Hᵤ represents the hypothesis that the individuals are unrelated. The related hypothesis was signified by a pedigree where the unidentified individual was tested as the relative. The unrelated hypothesis was signified by a Hardy-Weinberg equilibrium calculation. An LR value was calculated per locus and then multiplied across loci, which resulted in a final LR for the relationship. To improve computational efficiency, each locus LR was converted to logarithm and loci LRs were summed. However, due to the high plexity of this platform and the, often, high degradation level and/or low input of PM samples in mass fatality incident (MFI) cases, stochastic effects during PCR cause allele drop out and can introduce a situation where the allele combination between two individuals at a locus appear impossible. This typically occurs in parent-offspring relationships where each individual in the relationship is genotyped as homozygous for different alleles but one or both are actually heterozygous (e.g., parent has genotype AA and child as genotype BB when the parent or both parent and child are actually AB). Genotyping error and de novo mutation are also possible causes of such a case. This situation results in a LR of 0 and can result in an overall LR of 0 given that the final LR is the product of the loci LRs. To ensure locus LRs are not 0, a modification was made to pedprobr. The likelihood ratio for the locus in these cases was calculated as follows

$$L R = \frac{0.001}{{p}^{2} {q}^{2}}$$

where 0.001 represents a genotyping error rate, p is the allele frequency of the allele 1, and q is the allele frequency of allele 2 (Galván-Femenía et al., Heredity, 2021, 126:537-547, the content of which is hereby incorporated by reference in its entirety).

The whole genome kinship coefficient, shared cMs, and longest segment cMs were calculated using PC-Relate (Conomos et al., American Journal of Human Genetics, 2016, 98:127-148, the content of which is hereby incorporated by reference in its entirety) and PC-AiR (Conomos et al., Genetic Epidemiology, 2015, 39:276-293, the content of which is hereby incorporated by reference in its entirety) as described previously in Snedecor et al., Forensic Sci. Int. Genet., 2022, 61:102769, the content of which is hereby incorporated by reference in its entirety. This method is useful when the relationship between two individuals is unknown, thereby not requiring a pedigree.

The PC-AiR method first takes a set of genotyped individuals and separates them into two nonoverlapping subsets: one set containing unrelated individuals that represent ancestries of all individuals (unrelated subset), the other set containing individuals that have at least one relative within the first subset (related subset). To build the unrelated subset, a modification was made to the original PC-AiR method to improve computational efficiency in building the model. Samples with none or the fewest relatives are added to the unrelated subset, while those with more relatives are excluded from the unrelated subset. This is performed by calculating a kinship value as in Conomos et al., Genetic Epidemiology, 2015, 39:276-293, the content of which is hereby incorporated by reference in its entirety, per pair and categorizing each individual as having a relative or not based on stringent thresholds: a relative is considered if the kinship value is greater than 0.01 and not related if the kinship value is less than-0.025. Samples with less than 5% missing SNP data were excluded. Next, principal component analysis (PCA) was performed on the unrelated subset, then values were predicted along components of variations for all individuals in the related subset based on genetic similarities with individuals in the unrelated subset. The resulting components represented a model that can be used in place of static population frequencies to identify matches in a set of unknown individuals. The model used in this study was built using the GEDMatch database.

PC-Relate uses the principal components from PC-AiR and separates genetic correlations into two components: one for the sharing of alleles that are identical by descent from recent common ancestors and another for allele sharing due to more distant common ancestors. The components from PC-AiR were used to estimate allele frequencies based on the individual's ancestral background using linear regression instead of static population frequencies, such as those from gnoMAD. For two individuals, i and j, a kinship coefficient, [symbol] , was then calculated using the estimated allele frequencies, û, from the PC-AiR model as follows

$$\text{?} = \frac{{\sum }_{s ∈ S} ( {g}_{i s} - 2 {\hat{u}}_{i s} ) ( {g}_{j s} - 2 {\hat{u}}_{j s} )}{4 {{\sum }_{s ∈ S} [ {\hat{u}}_{i s} ( 1 - {\hat{u}}_{i s} ) \cdot {\hat{u}}_{j s} ( 1 - {\hat{u}}_{j s} ) ]}^{1 / 2}}$$

where s is a SNP in S SNPs that were typed in both individuals, gᵢₛ and gⱼₛ are the number of reference alleles in i and j at SNP s, respectively, and ûᵢₛ and ûⱼₛ are the expected allele frequencies calculated by PC-AiR for i and j at SNP s, respectively. This algorithm is termed “whole genome kinship,” as it considers the entire genome as one segment of relatedness, signified by a whole genome kinship coefficient. This whole genome kinship coefficient is used to identify relationships when referring to the whole genome kinship algorithm. When the number of SNPs shared between two individuals is less than 6000, a whole genome kinship coefficient of more than 0.031 between two individuals was required to be considered relatives in this study.

Snedecor et al., supra, introduced an additional step, termed “windowed kinship,” to identify distant relationships more accurately. Windowed kinship consists of calculating windows of kinship across the genome to find shared kinship segments. This Is performed by enumerating all possible windows within each chromosome and calculating a kinship coefficient for all windows. These windows are then filtered by a minimum kinship coefficient threshold and included in the shared cMs calculation. The filtered segments are then iterated and stretches of SNPs sharing at least one allele and two alleles are categorized separately. Total shared cMs is then calculated across all segments. Total shared cM and the longest segment of cM are used to identify relationships when referring to the windowed kinship algorithm. When the number of SNPs shared between two individuals is between 6000 and 8000, the shared cM value must be above 180 and the longest segment of cM must be above 30 to be considered a relationship. When the number of SNPs shared between two individuals is between 8000 and 9000, the shared cM value must be above 150 and the longest segment of cM must be above 30 to be considered a relationship. When the number of SNPs shared between two individuals is 9000 or more, the shared cM value must be above 140 and the longest segment of cM must be above 30 to be considered a relationship. The whole genome kinship coefficient can be used to filter at any number of SNPs shared. However, Snedecor et al., supra, observed a higher specificity when filtering on shared cM and longest segment cM (e.g., using windowed kinship) when the SNP overlap was greater than 6000, particularly for higher degrees of relationships.

More simply, the number of SNPs typed between two individuals (SNP overlap) can be used to decide when to use the whole genome kinship algorithm (<6000 SNPs overlap) and when to use the windowed kinship algorithm (>6000 SNPs overlap). And, when one algorithm is decided upon based on that SNP overlap, a value or a set of values are used to filter the data to identify relationships, depending on which algorithm was chosen. The cutoffs for both whole genome kinship and windowed kinship were chosen to ensure a high sensitivity but more importantly, a high specificity as demonstrated in Snedecor et al., supra. Lowering these thresholds may capture more relationships (i.e., increase sensitivity) but is expected to introduce more false positive hits, particularly for more distant relationships (e.g., fourth-and fifth-degree).

Using the above-described methods, a study was carried out to sequence libraries at higher plexity, thereby reducing total sample reads, in an effort to generate a stable but smaller set of typed SNPs for a cost-effective method for kinship analysis.

### B. Results

To show feasibility of high plexity using the sequence libraries, high plexity (>3 samples on a sequencing run) was simulated on a set of 30 libraries generated from high quality DNA samples from among the mock AM samples. Thirty Mock AM samples were prepared and sequenced together, as described above, to generate sample data with high numbers of reads. The BCL files were demultiplexed using a custom, local build of the ForenSeq UAS secondary analysis pipeline, which differs only in the generation of the FASTQ files due to differences in the raw data generated by the NextSeq. To determine the number of reads to simulate 16 sequence libraries, 25,000,000 (the total estimated number of reads) was divided by 16 (the number of samples) and multiplied by the average percent aligned (96%), resulting in 1,500,000 total reads per sample at this plexity. The same calculation was then performed to simulate 30 sequence libraries, but instead of dividing by 16, it was divided by 30, which resulted in 800,000 reads. Simulating a sequencing run with fewer reads is termed ‘downsampling’. Bioinformatic tools such as seqtk (https://github.com/lh3/seqtk) can be used to simulate a run with fewer reads or downsample the sequencing run. Seqtk was used to downsample each sample to either 1.5 million reads to simulate a plexity of 16 samples/run or 800,000 reads to simulate a plexity of 30 samples/run. seqtk randomly selects reads from the FASTQ files and outputs new FASTQs with the desired number of reads. The subsequent downsampled FASTQs were processed through the locally built ForenSeq UAS pipeline, which analyzed the FASTQs as described above.

The range of total reads per sample produced was 8,086,090 to 32,707,490 with an average of 23,186,251 reads. To simulate high plexity, reads from FASTQ files for each sample were randomly selected until a desired number of reads was met, which is termed downsampling. Downsampling is defined as the random selection of reads from a FASTQ file until the desired number of reads is achieved. The randomly chosen reads were subsequently output to a new FASTQ file. The resulting FASTQ files were analyzed with the bioinformatic algorithm as described above. Sequencing plexities of 16 and 30 were simulated by downsampling the data to 1.5 M reads and 800,000 reads for each sample, respectively. Decreasing the number of reads per sample resulted in the expected decrease in the number of typed SNPs.

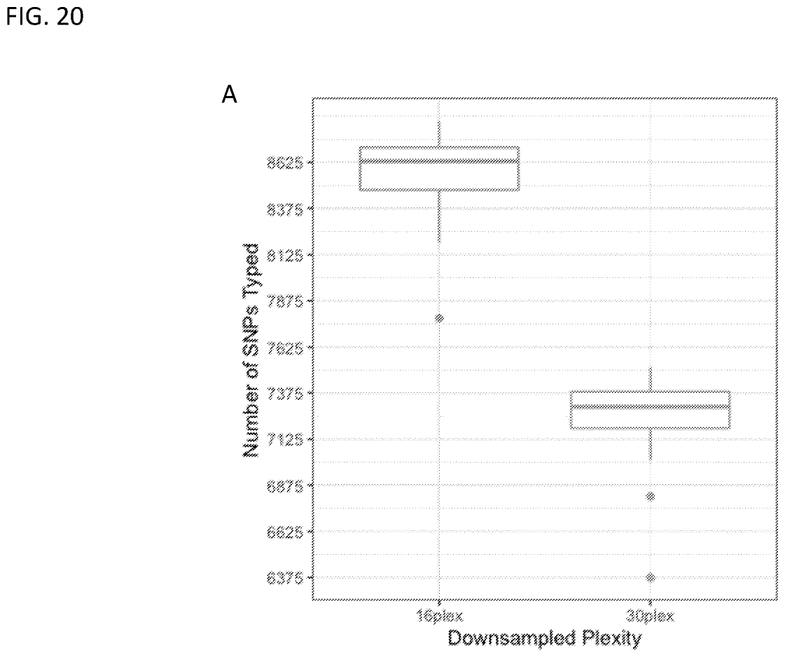

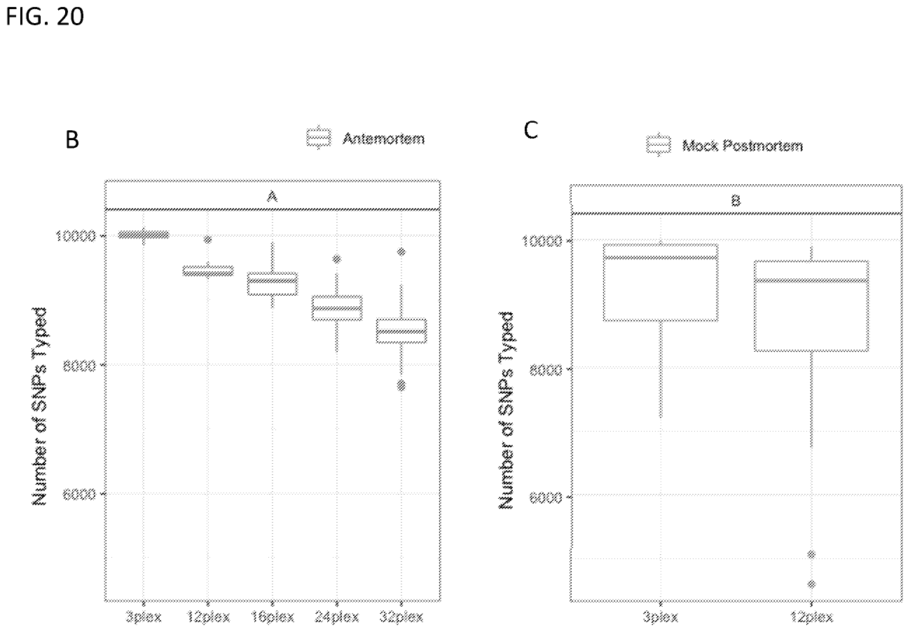

To determine the SNP call rate across samples, the number of typed SNPs was determined for each sample at each simulated sequencing plexity (16 and 30). 16 sample run (16plex) data was generated by downsampling the raw reads in the FASTQ files to 1.5 million reads per sample, while the 30-sample run (30plex) data was generated by downsampling the raw reads to 800,000 reads per sample. Subsequent genotyping was performed, and the number of SNPs typed per sample was summed. For 16-sample run, the minimum was 7781, the first quartile was 8472, the median was 8630, the third quartile was 8708, and the maximum was 8848. For the 30plex, the minimum was 6375, the first quartile was 7179, the median was 7299, the third quartile was 7382, and the maximum was 7516. The distribution of typed SNPs for the two simulated plexities for the 30 libraries is presented in FIG. 20A. The average recovery rate for the plexity of 16 was 8586 SNPs (ranging from 7781-8848 SNPs) with a median of 8630 SNPs, a first quartile of 8472 SNPs, and a third quartile of 8708 SNPs, and the average recover rate for the plexity of 30 was 7234 SNPs (ranging from 6375-7516 SNPs) with a median of 7299, a first quartile of 7179, and a third quartile of 7382 SNPs (FIG. 20A).

Determining relationships using the kinship algorithm described above and likelihood ratios rely significantly on the number of typed SNPs in each sample in the one-to-one comparison, also called the SNP overlap.

The higher the number of SNPs typed in both samples, the more confident the kinship and likelihood ratio values will be. Therefore, the average SNP overlap between the samples in both of the simulated 16 and 30 plexity sequencing runs was calculated to see if enough typed SNP loci would be shared between samples to identify true relationships with kinship and likelihood ratio values. The average common typed SNP loci across all combinations of samples in both of the simulated 16 and 30 plexity sequencing runs was 6998 loci, with a minimum overlap of 6058 and a maximum overlap of 7322. The simulations demonstrate that sequencing the libraries in such a way that fewer total reads are obtained for each sample will allow a smaller stable set of SNPs be typed for each sample with sufficient overlap for kinship determination. The number of common typed SNPs is less than 8000, which may not be sufficient to identify higher order relationships (e.g., fourth- and fifth-degree), but may be enough for identifying relationships to third-degree.

It was expected that sequencing libraries at an increased plexity would result in a lower number of typed SNPs, which could lead to elevated allele dropout and subsequent decreased heterozygosity, particularly for postmortem (PM) samples where the DNA is degraded and/or of low quantity. To evaluate the levels of locus and allele dropout and heterozygosity at higher plexities, libraries generated from mock antemortem samples (AM) and mock postmortem (PM) as described above were sequenced at the recommended plexity of 3 samples per sequencing run and then also at four higher plexities of 12, 16, 24, and 32 samples per sequencing run. The resulting numbers of typed SNPs are shown in FIG. 20B. As shown in FIG. 20B, the minimum, first quartile, median, third quartile, and maximum were as follows: 9853, 9976, 10009, 10059, and 10135 for the 3plex; 9332, 9394, 9419, 9520, and 9945 for the 12plex; 8881, 9091, 9303, 9419, and 9901 for the 16plex; and, 7653, 8348, 8515, 8706, and 9753 for the 32plex. As shown in FIG. 20C, the minimum, first quartile, median, third quartile, and maximum were as follows: 7215, 8677, 9724, 9923, and 9991 for the 3plex; and, 4603, 8261, 9360, 9664, and 9903 for the 12plex. As shown in FIG. 20B, the number of loci with reads below the accepted threshold (AT) increased as plexity increased for reference samples. Additionally, the distribution of loci dropping below the AT widened as the sequencing plexity increased with an average of 10,111 (minimum of 9,853, maximum of 10,135) SNPs typed for sample sequenced with 3 samples per run, and an average of 8,528 (minimum of 7,653, maximum of 9,753) SNPs typed for samples sequenced with 32 samples per run. The minimum number of SNPs typed remained above 7,000 at the highest plexity of 32 samples per sequencing run that was tested, which indicated that sequencing these libraries at high plexity results in higher numbers of typed SNP loci as compared to the simulated results discussed above and shown in FIG. 20A.

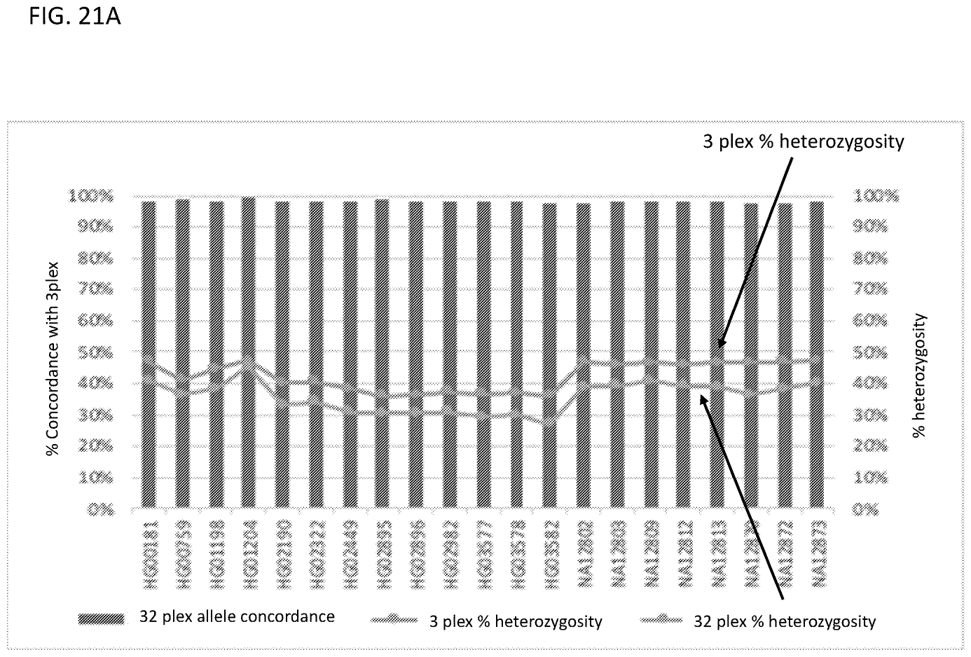

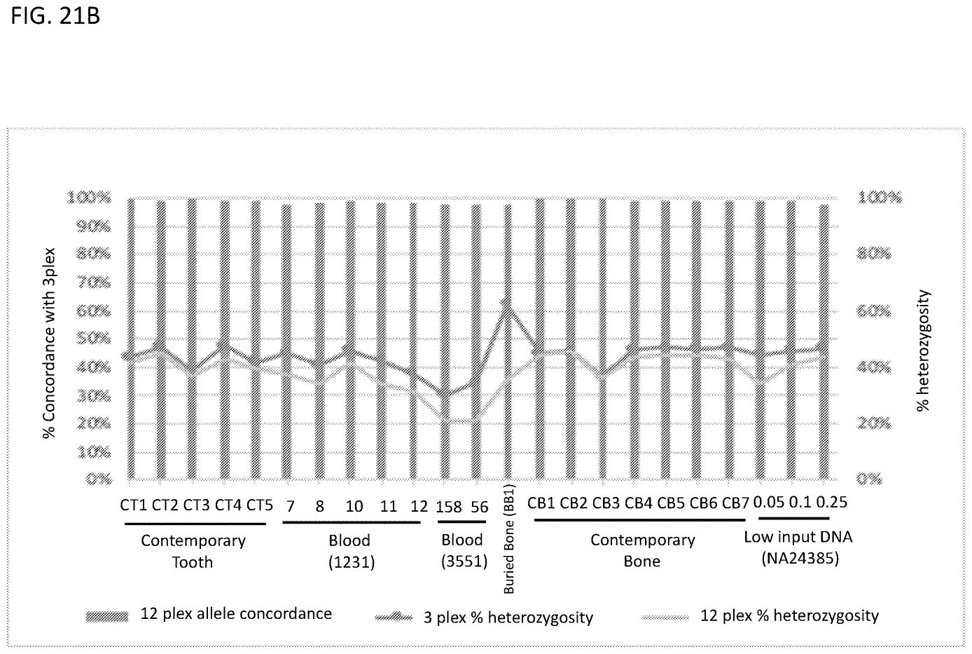

Next, the impact of higher plexity sequencing on loss of sister alleles for heterozygous loci was evaluated by comparing SNP genotypes determined for sample libraries sequenced at the recommended plexity of 3 samples per run to the SNP genotypes determined for the same sample libraries sequenced with 32 samples on the run. For each sample and at each locus that both the recommended plexity sequencing (3plex) and the higher plexity sequencing (32plex), genotypes were considered concordant if both runs typed the same alleles, and discordant otherwise. The average overlap between the 3plex and 32plex sequencing was 8,610 SNPs, with a minimum of 7,808 SNPs and maximum of 9,667 SNPs. For autosomal loci, both alleles must match to be considered concordant. Allele concordance for each sample was calculated by dividing the number of concordant alleles by the total number of alleles in the loci that were typed in both sequencing runs. For mock antemortem samples, allele discordance (alleles dropping below the AT) increased by an average of 1.9% between libraries sequenced at a plexity of 3 compared to 32, with a minimum of 0.50% and a maximum of 2.8% (FIG. 21A, left y-axis). Heterozygosity was determined by summing the number of heterozygous loci per sample and dividing that value by the total number of loci called. Samples sequenced with 32 samples per run (32plex) demonstrated the most difference in heterozygosity as compared to the standard plexity of 3 samples per run (3plex), with a 6.8% average difference, a 2.0% minimum difference, and a 10.2% maximum difference, with 3plex sequencing demonstrating greater heterozygosity by those values. (FIG. 21A, right y-axis).

A characteristic of samples from victims of MFIs is that they are often degraded and can contain low levels of genomic DNA. However, it is also advantageous in the context of cost effectiveness and throughout to increase the number of samples per sequencing run, although this can result in lower numbers of typed SNPs and decreased heterozygosity, thereby compounding these issues. To evaluate how increasing sequencing plexity compared to the standard protocol with 3 samples per sequencing run for degraded and low input DNA has an impact on mock postmortem samples, libraries were generated from 30 mock postmortem (PM) samples with varying levels of degradation, low input DNA samples, dental remains, and cremated, embalmed, burnt, and buried bones. These libraries were sequenced at the standard 3plex and at 12plex. The number of SNPs typed above AT per sample and per run were not significantly different between the two sequencing runs, although some samples sequenced at the higher plexity had a significantly lower number of SNPs typed. For instance, as shown in FIG. 20C, sequencing at 3plex resulted in an average of 9,313 SNPs, a minimum of 7,215 SNPs, and a maximum of 9,991 SNPs for the mock PM samples, whereas sequencing at 12plex resulted in an average of 8,796 SNPs, a minimum of 4,603 SNPs, and a maximum of 9,903 SNPs for the mock PM samples. These results indicate that increasing the number of samples per run for degraded and/or low DNA input samples, e.g., postmortem samples, will increase the number of loci below AT, but the biggest impact on numbers of typed loci is the degradation state and/or quantity of DNA.

Concordance of genotype calls for mock PM samples for libraries sequenced at 3plex or 12plex were run as described for the mock AM samples above. This included contemporary bone samples and contemporary tooth samples and, additionally, two series of degraded DNA (Samples 1231 and 3551) were purchased from Innogenomics Inc. (New Orleans, LA, USA). Percent concordance was calculated by comparing the 32plex or 12plex to the respective 3plex run and classifying the same allele calls at each locus as concordant, and different allele calls as discordant (below AT). Both alleles must match to be considered concordant for autosomal loci. A percentage was calculated by dividing the number of matching loci by the total number of loci that both runs called for each sample. The range of loci called for the mock AM samples in FIG. 21A was 7808 to 9667 with an average of 8610; and for the mock PM samples in FIG. 21B was 4580 to 9795 with an average of 8627. Percent heterozygosity was determined by summing the number of heterozygous loci and dividing that value by the total number of called loci. The mock PM samples were DNA extracted from dental remains (Tooth), blood of varying degradation levels (sample names starting with 1231 and 3551), a buried bone (BB1), a cremated bone (CB1), embalmed bones (CB2 and CB3), burnt bones (CB5, CB6, CB7), and a series of low DNA input of Coriell sample NA24385 at 0.05, 0.1, and 0.25 ng (50 pg, 100 pg, and 250 pg, respectively).

The number of loci that were typed in both runs ranged from 4580 to 9795 with an average of 8627 SNP loci. Allele discordance (alleles dropping below the AT) increased on average by 1.2% with a minimum of 0.1% and a maximum of 2.6% (FIG. 21B, right axis). The dental remains and the cremated and burnt bones demonstrated the lowest allele discordance, with 0.6% average discordance, 0.1% minimum discordance, and 1.3% maximum discordance.

Heterozygosity was calculated as above for the mock AM samples. Heterozygosity levels varied depending on the degradation level and amount of the input DNA (FIG. 21B, right axis). The two degraded DNA samples with degradation indices of 158 and 56 (3551_158 and 3551_56, respectively), and the interred bone sample (BB1) and the 0.05 ng (50 pg) NA24385 sample demonstrated the highest difference in heterozygosity between the sequencing plexities (9.0%, 13.5%, 26.9%, and 10.8%, respectively). The 1231 libraries 7-8, and 10-12 (FIG. 21B) were generated from the same DNA sample with increasing amounts of degradation (DIs of 26.3, 33.6, 48.6, 160.3, and 459.8 for 1231 samples designated as 7, 8, 10, 11, and 12, respectively). The heterozygosity decreased with increased degradation of the DNA, with 37% heterozygosity for sample 1231_7 with the DI of 26.3 and 31% heterozygosity for sample 1231_12 with the DI of 459.8 for these libraries sequenced at a plexity of 12 on the MiSeq FGx. BB1 had the highest level of heterozygosity when sequenced at 3plex. Investigation into the intra-locus balance (ILB) of this sample (BB1) demonstrated 53% and 28% ILB for the sample sequenced at a plexity of 3 samples and sequenced at a plexity of 12 samples per run, respectively. These results indicate that the cause of the increased heterozygosity observed for the BB1 sample sequenced at the plexity of 3 samples per run is not due to sequencing errors as the expectation would be that the ILB would be very low.

The bone samples with CB designations (burnt, embalmed, or cremated bone DNA extracts) demonstrated the smallest difference in heterozygosity (1.7% average difference; 0.1% minimum; 3.9% maximum among the seven samples). Additionally, high throughput using this approach performed well with the dental remains, which demonstrated a 2.5% average difference in heterozygosity with a minimum difference of 1.8% and a maximum difference of 4.5%.

To evaluate kinship of victims of MFIs, the AM sample was compared to the PM sample to test for relatedness, becoming a 1-to-1 comparison. The number of SNPs typed in both individuals (SNP overlap) significantly impacts the determination of relationships, particularly distant relationships, e.g., fourth and fifth degrees. Therefore, SNP overlap (SNP loci in common) was calculated among all combinations of samples between the mock PM samples sequenced at 12plex and the mock AM samples sequenced at 32plex. This was done by pairing each mock PM sample with each mock AM sample, identifying the number of SNP loci that were genotyped in both samples, and summing that value, resulting in a SNP overlap for that each pair. The average overlap between combinations of mock PM and mock AM samples was 8,020, with a minimum of 4,071 and a maximum of 9,727. These data indicate that, with an average around 8,000 SNPs, sequencing these libraries at higher plexities (12 for PM and 32 for AM samples per run) using this approach will be able to identify first, second, third, most fourth-degree relationships, and some fifth-degree relationships.

The ability of the whole genome kinship algorithm to identify relationships when the SNP call rate was <9000 loci in common (2000-8000 SNPs typed) was next investigated. Data presented by Snedecor et al., supra, demonstrated high accuracy of identifying all degrees of relationships when the SNP call rate was around 9,000 loci using the windowed kinship algorithm. Windowed kinship functions by calculating windows of kinship across the genome to find shared segments between two individuals. See Snedecor et al., supra. The whole genome kinship algorithm considers the entire genome as one segment of kinship instead of windows between two individuals. Snedecor et al. demonstrated that to identify higher order relationships (e.g., fourth- and fifth-degree), windowed kinship was more accurate when the number of SNPs typed in two corresponding samples (SNP overlap) was at least 9000 SNPs.

As shown in FIGS. 20B and 20C, mock AM and mock PM samples sequenced at high plexity were less likely to exhibit the required >/=9000 SNP call rate needed for the windowed kinship algorithm. As described in Snedecor et al., supra, when the SNP overlap is in the range of 6000-8000 loci in common, windowed kinship performed well for first-, second-, third-, and most fourth-degree relationships. However, when the range of SNP overlap is 2000-4000 loci in common, the performance of windowed kinship decreased.

Using the methods described above for simulating related profiles, the ability of the whole genome kinship algorithm to identify relationships when the SNP call rate was less than 9,000 loci in common (2,000 to 8,000 SNPs typed) was next investigated.

The windowed kinship algorithm reports relatedness with the metrics of shared centiMorgans (cMs) and longest segment cMs, whereas the whole genome kinship approach reports relatedness with the whole genome kinship coefficient, with the kinship coefficient threshold of >0.031 to be considered related.

1000 anonymized query profiles were chosen from the GEDMatch database at random and were searched across the database to select 2954 ‘related’ target profiles for relationships spanning first-to fifth-degree as well as unrelated samples with zero shared cMs. Briefly, a set of 1000 anonymized samples, termed “query samples”, were randomly selected from GEDMatch. These samples were then queried for relatives in the GEDMatch database and any hits, termed “target samples”, were selected based on shared centiMorgans (CM) values calculated by the GEDMatch one-to-many tool. This search resulted in 2954 target samples and, together with the matched query sample. These results included query-target pairs that had zero shared cMs, representing true unrelated pairs. Therefore, the number of target samples is larger than the number of query samples due to inclusion of both related and unrelated target samples. Relationship degree was determined by comparing the resulting shared cM values generated from the one-to-many tool to the expected range of shared cM per degree provided by DNA Painter. The profiles were first filtered for the 10,230 SNPs in the Kintelligence multiplex. Subsequently, the profiles were randomly filtered to 80%, 60%, 40%, and 20% call rates, resulting in 8000, 6000, 4000, and 2000 loci, respectively, in each profile for each query-target pair. The whole genome kinship algorithm was then calculated to test one-to-one relationships for every query-target pair at all SNP numbers for a total of 446,182 comparisons.

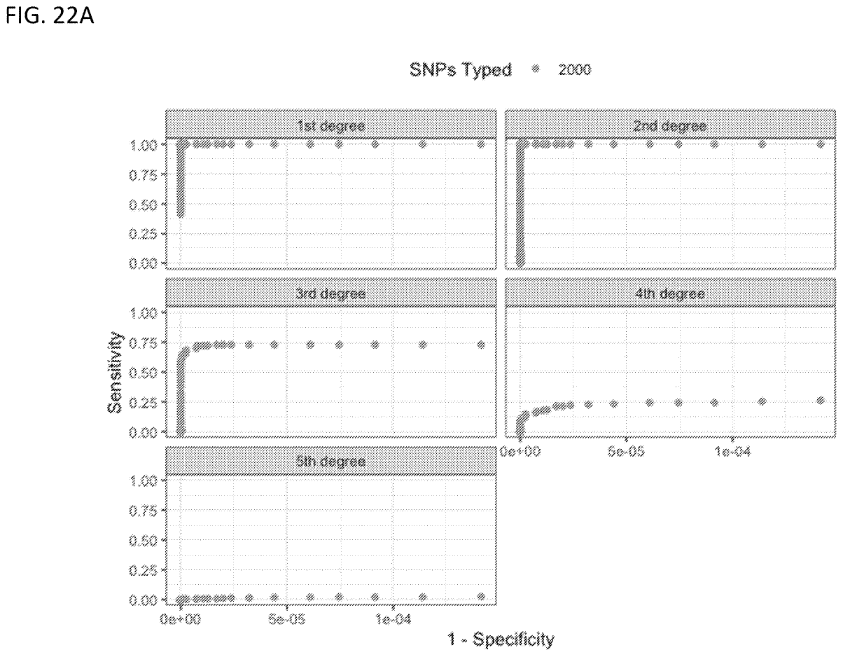

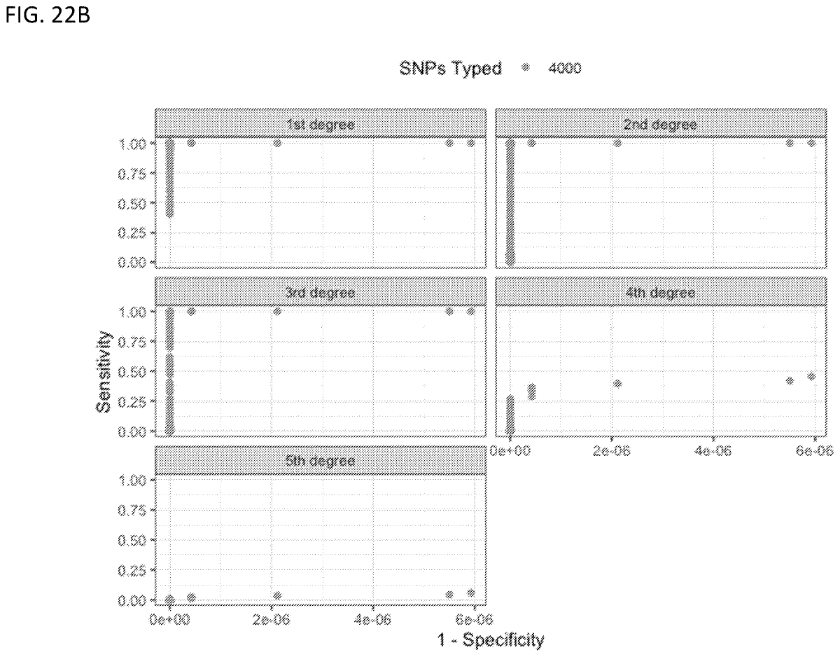

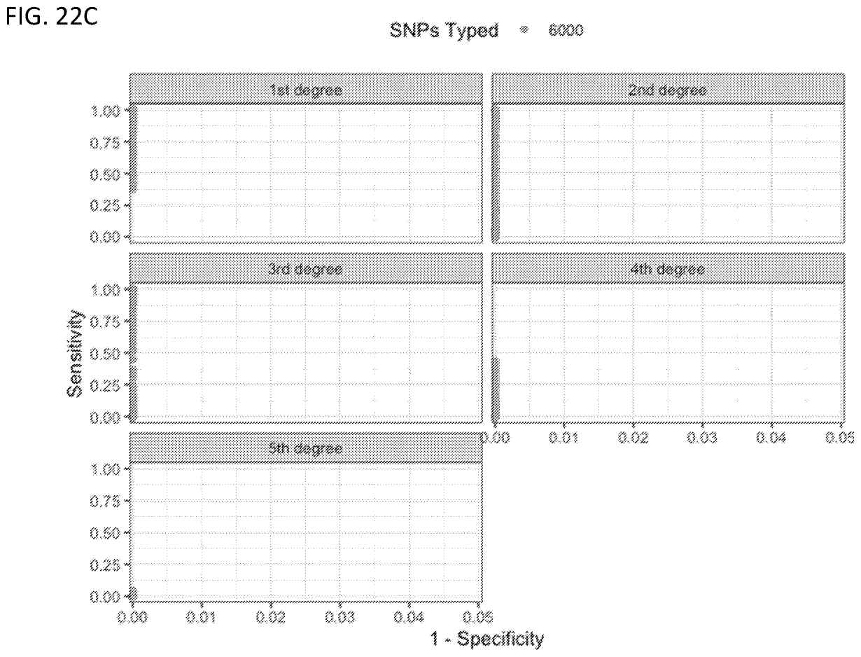

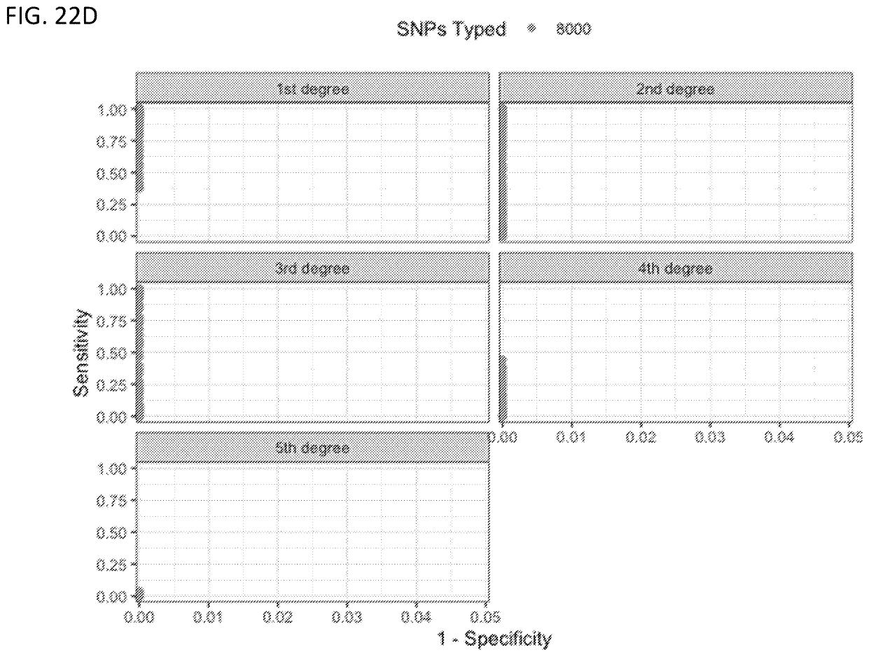

First and second-degree relationships exhibited a high specificity and sensitivity (FIGS. 22A-D), even for profiles with 2000 typed SNPs (average sensitivity of 70.6% and average specificity of 100%), as shown in FIG. 22A. Sensitivity began to decrease for third-degree relationships, but the whole genome kinship algorithm performed better for relationship detection (average sensitivity of 25.1%, average specificity of 99.2%) than windowed kinship for profiles with 4,000 to 8,000 SNPs typed (FIGS. 22B, C, and D). With fewer SNPs typed, fourth- and fifth-degree matches demonstrated low sensitivity and specificity (FIGS. 22A-D). The windowed kinship algorithm identified these higher order relationships with higher sensitivity and higher specificity with these library profiles with the high numbers of typed SNPs (Snedecor et al., Forensic Sci Int Genet 2022, 61, 102769, doi: 10.1016/j.fsigen.2022.102769, the content of which is hereby incorporated by reference in its entirety). Importantly, the specificity for all degrees remained above 99.98%, with 6,000 and 8,000 SNP ranges remaining above 99.9975% (FIG. 22C and FIG. 22D). These results indicate that whole genome kinship has a very low false positive rate. Overall, these data suggest that sequencing libraries as prepared herein at higher plexity will yield enough typed SNP data to confidently identify first-, second-, and third-degree relationships. These simultated results also suggests that coupling this approach for generating sequence libraries with the whole genome algorithm as described herein should work for disaster victim identification (DVI) and can be implemented on a local server (as opposed to a server accessible to the public and/or law enforcement agencies) to allow kinship determinations across small databases of antemortem samples.

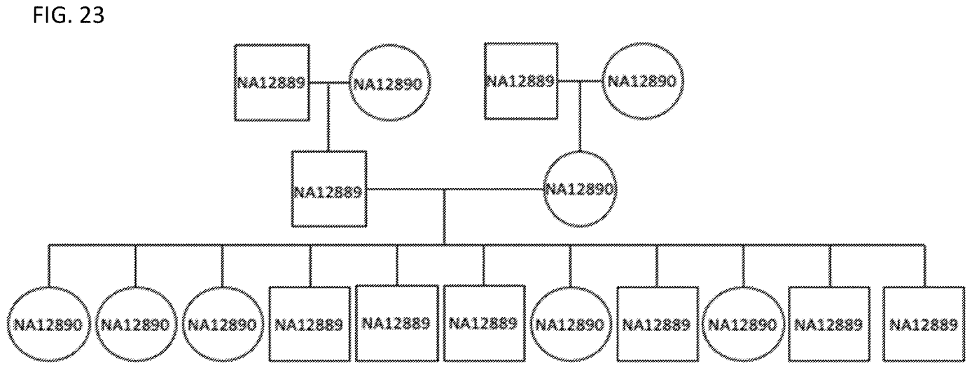

Next, the ability of the whole genome algorithm as described herein to identify true relationships on libraries as prepared by the methods described above was tested using the related antemortem CEPH/Utah samples as described above and shown in FIG. 23A. These samples were chosen because they include two sets of grandparents, one set of parents, and eleven siblings, thereby representing first and second degree relationships (FIG. 23A). These libraries were sequenced at plexities of 12, 16, 24, or 32 samples per sequencing run to determine the highest plexity that can maintain accuracy in identifying true relationships while excluding known unrelated pairs.

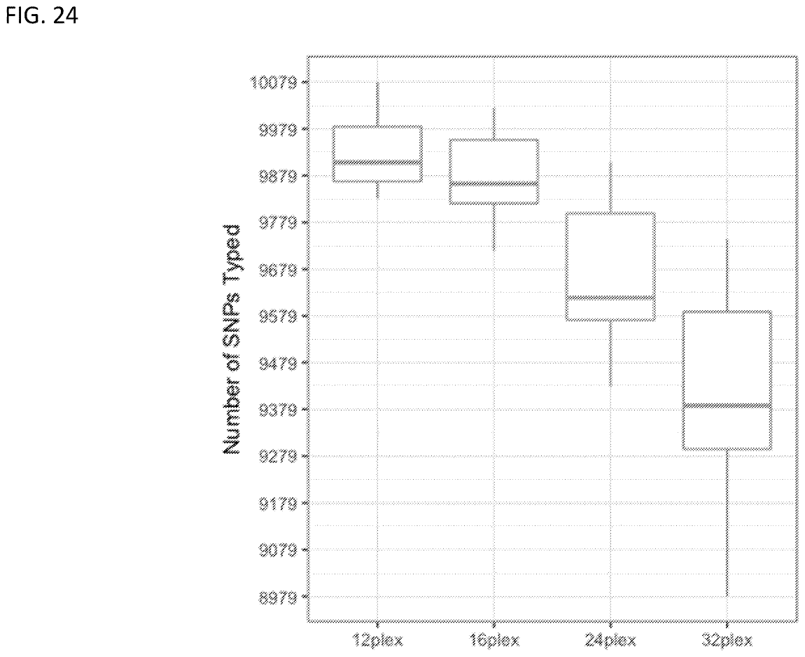

The number of SNPs typed in each sample per run was determined to ensure enough SNPs were typed to perform kinship and likelihood ratio calculations (FIG. 24). The SNP call rate decreased as the number of samples per run increased (FIG. 24), and the results were comparable to the initial high throughput testing in FIGS. 20B and 20C, thereby indicating the reproducibility of high plexity sequencing of these libraries. As shown in FIG. 24, the minimum, first quartile, median, third quartile, and maximum were as follows: 9831, 9859, 9907, 9987, and 10079 for the 12plex; 9718, 9816, 9862, 9957, and 10024 for the 16plex; 9428, 9570, 9618, 9798, and 9907 for the 24plex; 8979, 9294, 9387, 9588, and 9743 for the 32plex.

Kinship metrics were next calculated to confirm the sensitivity and specificity of the algorithms described herein using these high plexity profiles. The kinship metrics that were calculated include whole genome kinship coefficients, shared cM, longest peak cM, and likelihood ratios for all combinations of members in the pedigree. Most members in this family were related, with only five pairs of individuals being unrelated. To increase the number of unrelated controls and to best simulate databases used for missing persons cases (which are mostly composed of defined mock antemortem samples from family members of the victims), 100 randomly selected 1000 Genomes Project samples were included. Genotypes were downloaded from the International Genome Sample Resource database (Fairley et al., Nucleic Acids Res 2020, 48, D941-D947, doi: 10.1093/nar/gkz836, the content of which is hereby incorporated by reference in its entirety) and filtered to include only the loci in the 10,230 SNP panel of the ForenSeq Kintelligence Kit (Verogen, Inc.).

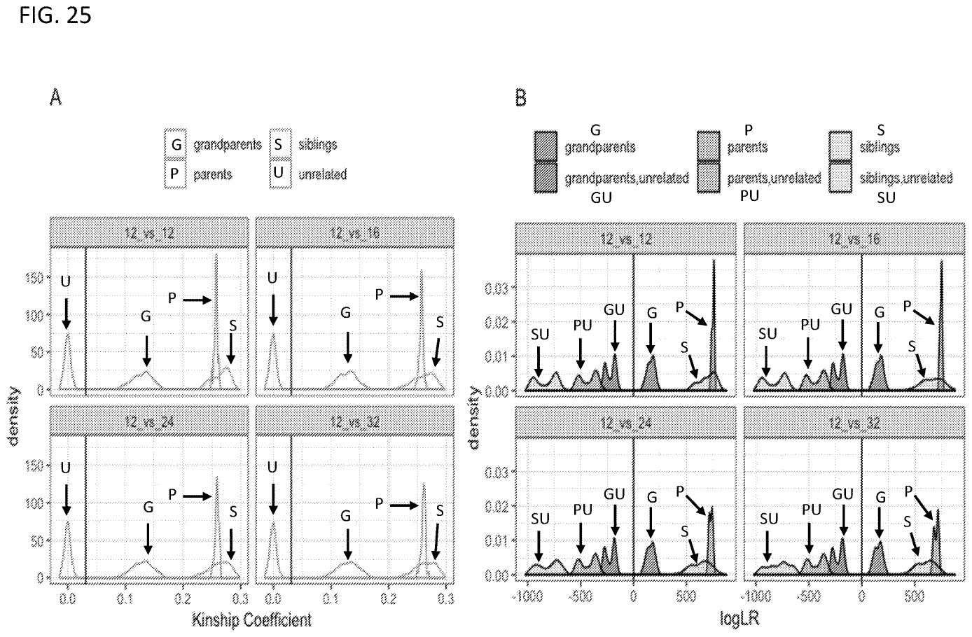

As demonstrated above, it was determined that a plexity of 12 samples per run was sufficient to call enough SNPs from mock postmortem (PM) samples that were degraded and/or have low quantities of DNA. To simulate a PM versus antemortem (AM) comparison as is performed for DVI, the 12-sample run samples were considered to be the PM samples. Each PM sample profile was then paired to each of the other sample profiles from all of the sequencing runs (12, 16, 24, and 32 samples per run) as well as the 100 unrelated profiles from the 1000 Genomes Project to calculate the whole genome kinship coefficient, shared cM, longest segment cM, and likelihood ratio for each pair to identify related pairs. The Utah/CEPH 1463 family includes grandparents, parents, and siblings. Each unique graph represents a comparison of the 12-sample run versus itself (12_vs_12), the 16-sample (12_vs_16), the 24-sample (12_vs_24), and 32-sample (12_vs_32) runs to simulate a postmortem vs antemortem comparison and to determine the highest plexity able to maintain accurate identification of close relationship for antemortem samples. Samples were paired and the kinship coefficient and log LR were calculated for each pair. For the kinship analysis of the Utah/CEPH 1463 family, 100 randomly selected samples starting with the HG prefix were included from the 1000 Genomes project (Fairley et al., Nucleic Acids Res 2020, 48, D941-D947, doi: 10.1093/nar/gkz836, the content of which is hereby incorporated by reference in its entirety) to serve as unrelated controls. Genotypes were downloaded from the International Genome Sample Resource database and filtered to include only the loci in the 10,230 Kintelligence SNP panel. A pair was considered related if the kinship coefficient was greater than 0.031, unrelated otherwise, which is represented by the black vertical line in FIG. 25A. A pair was considered related if the log LR was greater than 0, unrelated otherwise, which is represented by the black vertical line in FIG. 25B. The samples include samples from grandparents (G), parents (P), siblings(S), unrelated controls (U), unrelated grandparents (GU), unrelated parents (PU), and unrelated siblings (SU). As shown in FIGS. 25A and 25B, the kinship coefficient and log LR thresholds were sufficient to distinguish related from unrelated pairs.

Since the minimum SNP overlap was >8,900 among samples that were sequenced at all plexities, shared cM and longest segment cM were determined using the windowed kinship algorithm as described above. Using the whole genome algorithm as described above to determine relationships, kinship coefficients were well above 0.031 for all expected related pairs, and below 0.031 for all expected unrelated pairs. Moreover, since this family has a maximum of second-degree relationships, both windowed kinship and whole genome kinship performed equally well.

Whole genome kinship coefficients clearly discriminate each of the expected relationships with no false positive relationships detected (FIG. 25A). As shown in FIG. 25A, all unrelated pairs were below the threshold of 0.031 for the kinship coefficient, whereas all related pairs were well above the threshold of 0.031 for the kinship coefficient, thereby clearly distinguishing between related and unrelated pairs. All measures of kinship were able to accurately identify all relationships from the Utah/CEPH 1463 family, even when comparing profiles generated on the run with 12 samples to profiles generated on the run with 32 samples, given that all comparisons were performed within a small database such as those used in DVI cases, which contain few unrelated individuals. The likelihood ratios demonstrated a similar trend, with all expected related pairs correctly identified as related by having positive values above the threshold of 0, and all unrelated pairs being classified as unrelated by having negative values below the threshold of 0 (FIG. 25B). These data demonstrate that at a maximum plexity of 32 samples per run for mock AM libraries, profiles contain enough SNP data to accurately identify relationships up to second-degree and exclude unrelated pairs, including when the analysis is performed on a small database with few unrelated individuals, such as those used in missing persons and DVI cases.

Next, the related antemortem private family (“Related Family” or RF) samples, as described above and depicted in FIG. 26, were used to test higher order relationships, including first-, second-, third-, fourth-, and fifth-degree relationships, as shown in FIG. 26. This related private family included a self sample and individuals having particular relationships to the self sample, including parents (Father and Mother), an aunt, a first cousin (Cousin), a first cousin once removed (1C1R), and a second cousin (2^{nd} Cousin) (FIG. 26). These individuals labeled within FIG. 26with a relationship were sequenced and included in the following kinship analysis.

Three replicate libraries were generated for each of the 8 family members with several positive and negative amplification controls, and were sequenced with a total of 30 samples on the run.

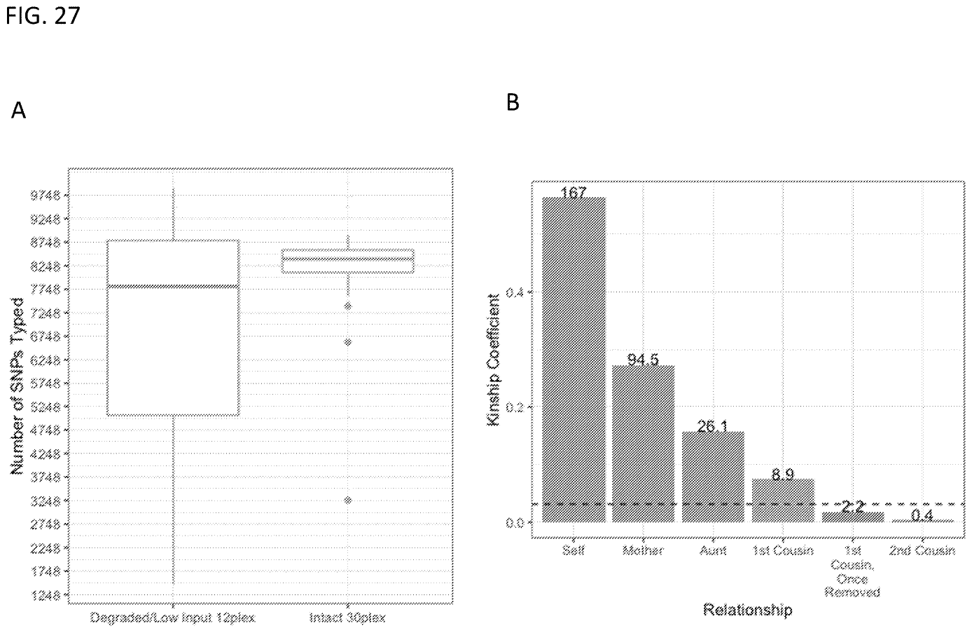

Replicates from Self (RF016) and Mother (RF004) were subjected to artificial degradation by temperature to mimic PM samples, specifically the samples were subjected to 21 cycles of heating and chilling at 98 °C for 1 hour followed by 4 °C for 10 minutes. The degradation index was measured and resulted in DIs of 2.1, 2.6, 5.1, and 20 for four replicates of Mother (RF004) and 1.5, 2.0, 2.2, and 2.9 for four replicates of Self (RF016). Libraries were generated from these degraded samples, as well as the intact Self and Mother DNA samples (50 pg). Degraded and low input samples were sequenced with 12 samples per run (12plex), and intact samples were sequenced at a plexity of 30) and show a higher number of typed SNPs with a tight distribution compared to the mock PM samples sequenced at a plexity of 12 where the number of typed SNPs were significantly lower and distributed across a wide range (FIG. 27A). The minimum, first quartile, median, third quartile, and maximum for these samples were as follows: 3256, 8106, 8391, 8580, and 8896 for the degraded/low input 12plex; and were 1470, 5057, 7807, 8782, and 9898 for the intact 30plex (FIG. 27A).

Furthermore, the mock AM sample profiles, libraries generated from intact DNA, had fewer typed SNPs compared to the mock AM sample profiles generated from commercially available DNA samples sequenced at a similar plexity (FIG. 27A compared to FIGS. 20B and 24). The average percent difference in heterozygosity between the intact mock AM Mother (RF004) samples sequenced at a plexity of 30 and corresponding degraded/low DNA input mock PM Mother (RF004) samples sequenced at a plexity of 12 was 14.6% with a minimum difference of 1.6% and a maximum of 25.5%.

One to one comparisons were performed by pairing each degraded/low input mock PM sample with each intact mock AM sample to calculate whole genome kinship coefficients, shared cM, longest segment cM, and likelihood ratios. All relationships up to and including third-degree were determined with 100% sensitivity and 100% specificity when using both the kinship algorithms described herein. Eight of the possible thirty fourth-degree relationships were identified with the windowed kinship algorithm described herein but only four of the possible thirty fourth-degree with the whole genome algorithm described herein, but no fifth-degree relationships passed the kinship thresholds with either of the algorithms. Thirty-nine of the total possible pairs of samples are unrelated within this data set and no false positive relationships were detected. 19 pairs of samples had less than 2,000 SNPs overlapping, 18 of which were mock PM sample Mother (RF004) with a DI of 2.6 and 7.2% heterozygosity and one of which was with mock PM sample Self (RF0016) with 50 pg DNA as input and 15.4% heterozygosity. Of these 19 pairs, seven were of first-degree, three were of second-degree, and three were of third-degree relationships that all had a kinship coefficient of over 0.031. Three pairs were self to self with high kinship coefficients. These results confirm that first-to third-degree relationships can be determined for mock PM and mock AM sample libraries sequenced at high plexity with high specificity using the methods described herein. An example of the whole genome kinship coefficient and the corresponding log likelihood ratios from one degraded mock PM sample (Self, RF016) that had a DI of 2.9, paired with an intact Self sample, and mock AM samples including the known Mother (first-degree), the known Aunt (second-degree), the known first Cousin (third-degree), the known first cousin once removed (fourth-degree), and the known second cousin (fifth-degree) is presented in FIG. 27B. Kinship of Self (RF016) (degradation index of 2.9) mock postmortem sample to the intact mock antemortem (AM) samples was determined using the whole genome algorithm. The horizontal dotted line depicted in FIG. 27B represents the kinship coefficient threshold of >0.031. The SNP overlap averaged at 7400 across the 6 pairs with a minimum of 7061 and a maximum of 7755 (FIG. 27B). The log likelihood ratios of the Self, Mother, Aunt, 1^{st} cousin, 1^{st} cousin once removed, and 2^{nd} cousin, were 167, 94.5, 26.1, 8.9, 2.2, and 0.4, respectively. All relationships except for first cousin once removed and second cousin (fourth- and fifth-degree) passed the required 0.031 thresholds for the windowed kinship and the whole genome algorithms using the methodologies as described herein, thereby confirming the effectiveness of using these algorithms for identifying first, second, and third degree relatives using the methods described herein.

### C. Discussion

Disaster victim identification (DVI) with DNA typically consists of interrogating highly polymorphic regions of the genome, specifically autosomal short tandem repeats (STRs), STRs on the Y chromosome, and mitochondrial DNA (mtDNA) (Alonso et al., Croat Med J 2005, 46, 540-548; Ambers et al., Int J Legal Med 2018, 132, 1545-1553; Alvarez-Cubero et al., Pathobiology 2012, 79, 228-238; Watherston et al., Forensic Sci Int Genet 2018, 37, 270-282; Prinz et al., Forensic Sci Int Genet 2007, 1, 3-12, the contents of which are hereby incorporated by reference in their entirety). STR markers have been used to identify the remains in several mass fatality incidences (MFIs) but can only provide enough information to identify first- and second-degree relationships and can result in a high false positive rate (Alonso et al., Croat Med J 2005, 46, 540-548; Alvarez-Cubero et al., Pathobiology 2012, 79, 228-238; Birus et al., Croat Med J 2003, 44, 322-326; Brenner et al., Theor Popul Biol 2003, 63, 173-178; Graham et al., Forensic Sci Med Pathol 2006, 2, 203-207, the contents of which are hereby incorporated by reference in their entirety). The development of next generation sequencing (NGS) assays for STR analysis have overcome some of the obstacles of utilizing STRs for DVI (Alvarez-Cubero et al., Ann Hum Biol 2017, 44, 581-592; Zavala et al., Impact of DNA degradation on massively parallel sequencing-based autosomal STR, iiSNP, and mitochondrial DNA typing systems, 2019; Senst et al., Forensic Science International: Genetics 2023, 62; Senst et al., J Forensic Sci 2022, 67, 1382-1398; Ambers et al., BMC Genomics 2016, 17, 750, the contents of which are hereby incorporated by reference in their entirety). The mtDNA control region, or hyper variable regions, or whole mitochondrial genome (mtGenome) have also been used to identify remains (Holland et al., Mitochondrial DNA Sequence Analysis—Validation and Use for Forensic Casework. Forensic science review 1999; Holland et al., Croat Med J 2003, 44, 264-272, the contents of which are hereby incorporated by reference in their entirety). The mtGenome has a high copy number and is a circular genome; therefore, there is a higher chance of recovery of mtDNA compared to nuclear DNA in fragmented, aged remains (Amorim et al., PeerJ 2019, 7, e7314, doi: 10.7717/peerj.7314, the content of which is hereby incorporated by reference in its entirety). However, mtDNA types the maternal lineage, which can be an issue if multiple family members are affected by an MFI or only paternally related relatives are available for kinship analysis (Zavala et al., Impact of DNA degradation on massively parallel sequencing-based autosomal STR, iiSNP, and mitochondrial DNA typing systems, 2019; Holland et al., Mitochondrial DNA Sequence Analysis-Validation and Use for Forensic Casework. Forensic science review 1999; Holland et al., Croat Med J 2003, 44, 264-272; Amorim et al., PeerJ 2019, 7, e7314, doi: 10.7717/peerj.7314. Conversely, single nucleotide polymorphisms (SNPs) can be used for kinship determination for either paternal or maternal lineage relationships or both (Watherston et al., Forensic Sci Int Genet 2018, 37, 270-282; Amorim et al., Forensic Sci Int 2005, 150, 17-21; Gorden et al., Forensic Sci Int Genet 2022, 57, 102636). Furthermore, for PCR-based assays, SNP amplicons tend to be shorter than those for STRs and are more likely to be amplified in compromised samples (Watherston et al., Forensic Sci Int Genet 2018, 37, 270-282; Zavala et al., Impact of DNA degradation on massively parallel sequencing-based autosomal STR, iiSNP, and mitochondrial DNA typing systems. 2019; Senst et al., J Forensic Sci 2022, 67, 1382-1398; Ambers et al., BMC Genomics 2016, 17, 750, the contents of which are hereby incorporated by reference in their entirety). And, with a high number of SNPs being interrogated, kinship and especially likelihood ratio values will be more discriminatory than for STRs (Turner et al., Front Genet 2022, 13, 882268; Cho et al., Transfus Med Hemother 2016, 43, 429-432; Tillmar et al., Genes 2021, 12, 1968). Although STRs are more polymorphic than SNPs (Devesse et al., Forensic Sci Int Genet 2018, 34, 57-61; Phillips et al., ELECTROPHORESIS 2018, 39, 2708-2724; Gettings et al., Forensic Science International: Genetics 2016, 21, 15-21; Novroski et al., Forensic Sci Int Genet 2016, 25, 214-226; Wendt et al., Forensic Sci Int Genet 2017, 28, 146-154; Delest et al., Forensic Sci Int Genet 2020, 47, 102304, the contents of which are hereby incorporated by reference in their entirety), SNP assays interrogate hundreds to thousands of data points compared to tens of data points with STRs. The high density of SNP data can reduce the false positive rate (Snedecor et al., Forensic Sci Int Genet 2022, 61, 102769, the content of which is hereby incorporated by reference in its entirety). Finally, SNPs have been used previously in situations of degraded DNA and proven to provide enough discriminatory power to identify remains (Snedecor et al., Forensic Sci Int Genet 2022, 61, 102769; Gorden et al., Forensic Sci Int Genet 2022, 57, 102636; Marshall et al., Genes (Basel) 2020, 11, doi: 10.3390/genes11080938, the contents of which are hereby incorporated by reference in their entirety).

The ForenSeq Kintelligence Library Prep Kit® includes a set of 10,230 forensically relevant SNPs that can be used to solve violent crimes and missing persons cases utilizing forensic genetic genealogy (Kling et al., Forensic Sci Int Genet 2021, 52, 102474; Snecedor et al., Forensic Science International: Genetics 2022, 61, 102769; Peck et al., Internal Validation of the ForenSeq Kintelligence Kit for Application to Forensic Genetic Genealogy. bioRxiv 2022, 2022.2010.2028.514056, doi: 10.1101/2022.10.28.514056; Verogen. ForenSeq Kintelligence Kit Datasheet: Document #VD2020054. Rev. A. 2021. Available online: https://verogen.com/wp-content/uploads/2021/03/forenseq-kintelligence-datasheet-vd2020054-a.pdf; Verogen. High-Quality Outcomes from Low-Quality Samples with ForenSeq Kintellingence Application Note: Document #VD2021002 Rev. B. 2021. 2021, the contents of which are hereby incorporated by reference in their entirety). ForenSeq Kintelligence has been used to solve an unidentified remains case in Oregon state (International, D.L. DNA Labs International is the first accredited lab to use the ForenSeq Kintelligence System to aid in the identification of unidentified remains. 2022). Kintelligence results can be uploaded to a database, such as GEDMatch PRO or FamilyTreeDNA (Verogen. Verogen and Gene by Gene Form Groundbreaking Partnership to Accelerate Adoption for Forensic Investigative Genetic Genealogy. Available online: https://www.businesswire.com/news/home/20220815005116/en/Verogen-and-Gene-by-Gene-Form-Groundbreaking-Partnership-to-Accelerate-Adoption-of-Forensic-Investigative-Genetic-Genealogy) to search for unknown relatives available in the database. The algorithm utilized in GEDMatch PRO was specifically designed to work with the 10,230 SNPs in the Kintelligence kit but requires upload to the public database to search for relatives and a minimum of 6000 SNPs typed in the sample is required for upload. Additionally, the current configuration of the Kintelligence kit allows for a maximum of 3 samples to be sequenced at a time on the Miseq FGx, ensuring enough SNPs are typed for GEDMatch PRO upload but also reducing the cost effectiveness for MFI cases where distant relationships are not desired. Furthermore, if a relative is known but does not exist in one of the two databases, matching postmortem (PM) samples would require known relatives to upload their profiles to the databases.

To assist with utilization of library prep kits and similar methodologies for nucleic library generation, such as the ForenSeq Kintelligence Library Prep Kit®, for DVI in a higher throughput and cost-effective manner, libraries were sequenced at plexities exceeding the recommended plexity of 3 libraries per sequencing run. The goal was to maximize plexity for both antemortem (AM) and postmortem (PM) samples while maintaining accuracy in identifying relationships up to third-degree.

The optimal plexity of sequencing for up to third-degree relationship determinations was 12 mock PM libraries or 32 mock AM libraries per sequencing run based on simulations (FIG. 20A) and sequencing mock AM and PM samples on the MiSeq FGx (FIGS. 20B, 20C, 24, and 27A). Although SNP alleles and loci fell below the AT for libraries sequencing at the higher plexities compared to the recommended plexity of 3 samples per sequencing run (FIGS. 21A and 21B), enough loci were typed in common between the mock AM and PM samples to allow up to third-degree relationships to be determined even for highly degraded and low DNA input samples (FIGS. 22, 25A, 25B, and 27B).

The analysis performed with the Related Family (RF) confirmed identifying relationships to third-degree, in addition to identifying some fourth-degree relationships (FIG. 27B). The SNP overlaps between samples with fourth-degree relationships ranged from 1356-7872 SNPs. Of the 30 possible pairs, eight pairs with an overlap of greater than 7000 SNPs did not pass the thresholds for kinship determination. If the quality of the PM samples is good enough to maintain a high SNP call rate, fourth-degree relationships may be determined but may not pass the thresholds (FIGS. 22A-D).

With lowered SNP call rates and increased allele dropout observed in the mock PM analysis as described in the study pertaining to FIGS. 21A and 21B, as well as the analysis of the degraded Related Family samples as described in the study pertaining to FIG. 27A with the pedigree as shown in FIG. 26, the overlap of loci called between PM samples and AM samples will decrease as the quality of the PM DNA decreases. This overlap significantly affects the performance of the kinship algorithms reducing sensitivity at the higher degrees of relationship (FIGS. 22A-D). Therefore, the number of SNPs typed in AM samples must be maximized to prevent further loss of loci. If more distant relationships are expected in a search of a MFI and PM samples exhibit a low SNP call rate, these libraries can be resequence at a lower plexity to ensure enough SNPs are called.

The high throughput simulations performed in the context of FIG. 20A and the real high throughput analysis performed in the context of FIGS. 20B and 20C demonstrated that increasing plexity will decrease the number of SNPs typed. Higher levels of degradation and lower amounts of DNA for PM samples will further affect the number of SNPs typed. As demonstrated the study where anonymized relatives from the GEDMatch database were sampled and kinship was determined by randomly reducing the number of SNPs to 8000, 6000, 4000, and 2000, whole genome kinship (using the kinship coefficient to identify relationships instead of shared cM) was able to identify up to third-degree with a high level of specificity and sensitivity (FIGS. 22A-D). Lowering the kinship coefficient threshold would improve the sensitivity (number of possible matches); however, the specificity would suffer, and more false positives would arise (the number of false matches that would be called true matches). The thresholds used in the current study resulted in a higher specificity (nearly 100%), favoring loss of true positives to avoid an increase in false positives. Additionally, at these lower SNP call rates, identifying more distant relationships such as those at fourth- and fifth-degree, becomes difficult. Both whole genome kinship and windowed kinship algorithms are sensitive to the number of SNPs typed between two individuals being tested. In the Related Family analysis, which included both degraded and low input samples, we were able to identify all relationships up to and including third-degree relationships. However, because the average number of SNPs typed was too low to refer to windowed kinship (less than 6000) for fourth-and fifth-degree relationships, we were unable to utilize the windowed kinship algorithm which is more specific for these more distant relationships. The whole genome kinship algorithm is not as specific or sensitive as the windowed kinship algorithm at identifying fourth- and fifth-degree relationships.

Heterozygosity is another measure of data dropout, which can affect the accuracy of likelihood ratios and kinship values if too low. Loss of heterozygosity occurs due to the high degradation in samples or low quantity of DNA. Even with a degraded sample exhibiting 7.2% heterozygosity observed in the Related Family samples, all first-, second- and third-degree relationships were captured. Fourth-degree relationships were determined with a mock PM sample exhibiting 22.6% heterozygosity. Together with the level of locus dropout observed in degraded and low input samples, it is expected that increasing the number of samples that can be sequenced concurrently to 12 for degraded and/or low DNA input samples and 32 for AM samples would be able to capture all relationships up to and including third-degree relationships.

If PM samples are expected to be fourth- or fifth-degree relatives, maximizing the SNP overlap by decreasing the number of samples sequenced concurrently is recommended, especially if the input is low and/or the sample is highly degraded. Alternatively, if PM samples are expected to be first-, second- or third-degree relatives, a higher number of samples sequenced concurrently (up to 12) even for degraded and/or low input samples should not affect the ability of kinship to identify first-to third-degree relationships. Alternatively, for AM samples, as stated above, the number of SNPs typed must be maximized; however, it was observed that up to 32 samples sequenced concurrently can identify up to and including all third-degree relationships, with both the described kinship algorithm and likelihood ratios.

In conclusion, sequencing libraries at high multiplexy in accordance with the methodologies described herein improves the cost effectiveness of relationship identification by increasing the multiplexity from 3 to 12 for PM samples and up to 32 for AM samples. Highly tuned thresholds set in the algorithms, and a large panel of SNPs reduce the false positive rate in identifying relationships to zero while identifying, with perfect sensitivity and specificity, all relationships up to third-degree (e.g., first cousin or great grandparent). These kinship algorithms, installed on a private server with the additional method for likelihood ratio calculations, served as a private database with the ability to find relatives and correctly determine relationships for MFI coupled with the cost-effectiveness of higher multiplexed sequencing is a solution for DVI.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

What is claimed is:

1. A method for performing DNA-based kinship analysis, comprising:
providing a nucleic acid sample from a person of interest,
amplifying the nucleic acid sample with a plurality of primers that specifically hybridize to a plurality of target sequences collectively comprising a plurality of at least between at or about 2,000 to 50,000 single nucleotide polymorphisms (SNPs), thereby generating amplification products, wherein the amplification is carried out in one or more multiplex PCR reactions,
generating a nucleic acid library from the amplification products,
sequencing the nucleic acid library generated from the amplification products,
analyzing the sequences of the amplification products,
determining the genotypes of the plurality of SNPs, thereby generating a DNA profile, and
calculating the degree of relationship of the DNA profile to one or more reference DNA profiles, wherein the one or more reference DNA profiles are comprised within a reference set of DNA profiles comprising one or more reference DNA profiles from a relative of the person of interest.

2. A method for performing DNA-based kinship analysis, comprising:
providing a nucleic acid sample from a person of interest,
amplifying the nucleic acid sample with a plurality of primers that specifically hybridize to a plurality of target sequences collectively comprising a plurality of at least between at or about 2,000 to 50,000 single nucleotide polymorphisms (SNPs), thereby generating amplification products, wherein the amplification is carried out in one or more multiplex PCR reactions,
generating a nucleic acid library from the amplification products,
sequencing the nucleic acid library generated from the amplification products,
determining the genotypes of the plurality of SNPs, thereby generating a DNA profile, and
calculating the degree of relationship of the DNA profile to one or more reference DNA profiles, wherein the one or more reference DNA profiles are comprised within a reference set of DNA profiles comprising one or more reference DNA profiles from a relative of the person of interest.

3. The method of claim 1 or claim 2, wherein the sequencing is conducted using massively parallel sequencing (MPS).

4. The method of any one of claims 1-3, wherein the sequencing does not comprise whole genome sequencing (WGS).

5. The method of any one of claims 1-4, further comprising generating a family tree comprising the DNA profile in relation to one or more DNA profiles.

6. A method of constructing a nucleic acid library for a person of interest, comprising:
providing a nucleic acid sample from a person of interest,
amplifying the nucleic acid sample with a plurality of primers that specifically hybridize to a plurality of target sequences collectively comprising a plurality of at least between at or about 2,000 to 50,000 single nucleotide polymorphisms (SNPs), thereby generating a nucleic acid library comprising amplification products, wherein the amplification is carried out in one or more multiplex PCR reactions.

7. The method of claim 6, further comprising a step of sequencing the amplification products to produce a DNA profile for the person of interest.

8. A method of constructing a nucleic acid library for a reference DNA sample, comprising:
providing a nucleic acid sample from a relative of a person of interest,
amplifying the nucleic acid sample with a plurality of primers that specifically hybridize to a plurality of target sequences collectively comprising a plurality of at least between at or about 2,000 to 50,000 single nucleotide polymorphisms (SNPs), thereby generating a nucleic acid library comprising amplification products, wherein the amplification is carried out in one or more multiplex PCR reactions.

9. The method of claim 8, wherein the relative is a first-, second-, third-, fourth-, or fifth-degree relative of the person of interest.

10. The method of claim 8 or claim 9, wherein the relative is a first-, second-, or third-degree relative of the person of interest.

11. The method of any one of claims 1-10, wherein the nucleic acid sample comprises genomic DNA.

12. The method of any one of claims 1-11, wherein the nucleic acid sample comprises one or more enzyme inhibitors.

13. The method of claim 12, wherein the one or more enzyme inhibitors comprise one or more inhibitors selected from the group consisting of hematin, heme, humic acid, indigo, tannic acid, collagen, calcium, and hydroxyapatite.

14. The method of any one of claims 1-13, wherein the nucleic acid sample comprises low-quality nucleic acid molecules and/or low quantity nucleic acid molecules.

15. The method of claim 14, wherein the low quality nucleic acid molecules are degraded genomic DNA and/or fragmented genomic DNA.

16. The method of claim 14 or claim 15, wherein the low quality nucleic acid molecules have a degradation index (DI) of at or at least 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200.

17. The method of claim 14 or claim 15, wherein the low quality nucleic acid molecules have a DI of at least 1 and up to or less than 158.3.

18. The method of any one of claims 1-13, wherein the nucleic acid sample comprises high quality nucleic acid molecules.

19. The method of claim 18, wherein the high quality nucleic acid molecules have a DI of less than 1.

20. The method of any one of claims 1-19, wherein the person of interest is a missing person.

21. The method of any one of claims 1-19, wherein the person of interest a victim of a disaster or conflict.

22. The method of any one of claims 1-21, wherein the nucleic acid sample is derived from saliva, blood, semen, hair, teeth, bone, or skin.

23. The method of claim 22, wherein the nucleic acid sample is derived from saliva, blood, or semen.

24. The method of claim 22, wherein the nucleic acid sample is derived from bone or hair.

25. The method of any one of claims 1-21, wherein the nucleic acid sample is derived from a buccal swab, paper, fabric, or other substrate or object that is impregnated with saliva, blood, semen, or other bodily fluid, or contains hair or skin cells.

26. The method of any one of claims 1-25, wherein the nucleic acid sample comprises between or between about 3 pg and 100 ng of genomic DNA.

27. The method of any one of claims 1-26, wherein the nucleic acid sample comprises between or between about 100 pg and 5 ng of genomic DNA, between or between about 50 pg and 5 ng of genomic DNA, or between or between about 3 pg and 5 ng of genomic DNA.

28. The method of claim 26 or claim 27, wherein the nucleic acid sample comprises at or about 1 ng of genomic DNA.

29. The method of any one of claims 1-28, wherein the plurality of SNPs comprises kinship SNPs (kiSNPs).

30. The method of any one of claims 1-29, wherein the plurality of SNPs comprises Y-chromosome SNPs (Y-SNPs).

31. The method of any one of claims 1-30, wherein the plurality of SNPs comprises kiSNPs and Y-SNPs.

32. The method of any one of claims 1-31, wherein the plurality of SNPs comprises kiSNPs, biogeographical ancestry SNPs (aiSNPs), identity SNPs (iiSNPs), phenotype SNPs (piSNPs), X-chromosome SNPs (X-SNPs), and Y-chromosome SNPs (Y-SNPs).

33. The method of any one of claims 1-28, wherein the plurality of SNPs comprises SNPs selected from one or more of the groups consisting of kiSNPs, aiSNPs, iiSNPs, piSNPs, X-SNPs, and Y-SNPs.

34. The method of any one of claims 1-33, wherein at least or at least about 80%, 81%, 82%, 83%, 84%, 85%, 86%, 87%, 88%, 89%, 90%, 91%, 92%, 93%, 94%, 95%, 96%, 97%, 98%, or 99% of the plurality of SNPs are kinship SNPs.

35. The method of any one of claims 1-34, wherein the reference set of DNA profiles comprises up to 5, 10, 15, 20, 25, 30, 35, 40, 45, 50, 55, 60, 65, 70, 75, 80, 85, 90, 95, 100, 125, 150, 175, 200, 300, 400, 500, 600, 700, 800, 900, or 1000 reference DNA profiles.

36. The method of any one of claims 1-35, wherein at least 5%, 10%, 15%, 20%, 25%, 30%, 35%, 40%, 45%, 50%, 55%, 60%, 65%, 70%, 75%, 80%, 85%, 90%, or 95% of the reference DNA profiles in the reference set of DNA profiles is from a relative of the person of interest.

37. The method of any one of claims 1-36, wherein at least 5%, 10%, 15%, 20%, 25%, 30%, 35%, 40%, 45%, 50%, 55%, 60%, 65%, 70%, 75%, 80%, 85%, 90%, or 95% of the reference DNA profiles in the reference set of DNA profiles is from a relative of the person of interest, and wherein each of the at least 5%, 10%, 15%, 20%, 25%, 30%, 35%, 40%, 45%, 50%, 55%, 60%, 65%, 70%, 75%, 80%, 85%, 90%, or 95% of the reference DNA profiles in the reference set of DNA profiles is a first degree, second degree, third degree, fourth degree, or fifth degree relative.

38. The method of any one of claims 1-37, wherein at least 50% of the reference DNA profiles in the reference set of DNA profiles is from a relative of the person of interest.

39. The method of any one of claims 36-38, wherein each relative of the person of interest in the reference set of DNA profiles is individually a first degree, second degree, third degree, fourth degree, or fifth degree relative of the person of interest.

40. The method of claim 39, wherein each relative of the person of interest in the reference set of DNA profiles is individually a first degree, second degree, or third degree relative of the person of interest.

41. The method of any one of claims 1-40, wherein the identity of each relative of the person of interest in the reference set of DNA profiles is known.

42. The method of any one of claims 1-41, wherein the identity of each of the one or more reference DNA profiles in the reference set of DNA profiles is known.

43. The method of any one of claims 1-42, wherein the reference set of DNA profiles is in a database.

44. The method of claim 43, wherein the database is not publicly accessible.

45. The method of any one of claims 1-44, wherein the sequencing comprises a sequencing plexity of up to 40-plex.

46. The method of any one of claims 1-44, wherein the sequencing comprises a sequencing plexity of up to 32-plex.

47. The method of any one of claims 1-44, wherein the sequencing comprises a sequencing plexity of 12-plex to 32-plex.

48. The method of any one of claims 1-44, wherein the sequencing comprises a sequencing plexity of 24-plex to 32-plex.

49. The method of any one of claims 1-44, wherein the sequencing comprises a sequencing plexity of at or about 10-plex, 11-plex, 12-plex, 13-plex, 14-plex, 15-plex, 16-plex, 17-plex 18-plex, 19-plex, 20-plex, 21-plex, 22-plex, 23-plex, 24-plex, 25-plex, 26-plex, 27-plex, 28-plex, 29-plex, 30-plex, 31-plex, 32-plex, 33-plex, 34-plex, or 35-plex.

50. The method of any one of claims 1-49, wherein the sequencing comprises a sequencing plexity of at or about 8-to 16-plex for postmortem samples, and/or the sequencing comprises a sequencing plexity of at or about 24-to 40-plex for antemortem samples.

51. The method of any one of claims 1-50, wherein the sequencing comprises a sequencing plexity of at or about 12-plex for postmortem samples, and/or the sequencing comprises a sequencing plexity of at or about 32-plex for antemortem samples.

52. The method of any one of claims 1-51, wherein the sequencing comprises a sequencing plexity of at or about 30-plex, 31-plex, or 32-plex.

53. The method of any one of claims 1-52, further comprising identifying the person of interest.

54. A method for calculating degree of relatedness, comprising:
obtaining a DNA profile comprising genotypes of at least between at or about 2,000 to 50,000 SNPs, wherein the DNA profile is from a person of interest; and
calculating the degree of relationship of the DNA profile to one or more reference DNA profiles, wherein the one or more reference DNA profiles are comprised within a reference set of DNA profiles comprising one or more reference DNA profiles from a relative of the person of interest.

55. A method for calculating degree of relatedness, comprising:
generating a DNA profile comprising genotypes of at least between at or about 2,000 to 50,000 SNPs, wherein the DNA profile is from a person of interest; and
calculating the degree of relationship of the DNA profile to one or more reference DNA profiles, wherein the one or more reference DNA profiles are comprised within a reference set of DNA profiles comprising one or more reference DNA profiles from a relative of the person of interest.

56. The method of any one of claims 1-55, wherein the degree of relationship is calculated using a kinship model.

57. The method of any one of claims 1-56, wherein the degree of relationship is calculated using a kinship model that is trained using a PCA method.

58. The method of claim 57, wherein the PCA method for training the kinship model is PCA or involves PCA.

59. The method of claim 57 or claim 58, wherein the PCA method is PC-AiR.

60. The method of claim 59, wherein the PC-AiR comprises the steps of: (1) estimating kinship coefficients between all pairs of samples, optionally training DNA profiles, of a training database, wherein pairings with a kinship coefficient >0.025 are identified as related and pairings with a kinship coefficient <−0.025 are identified as ancestry-diverged; (2) initializing an unrelated sample set that includes all samples; and (3) iteratively: (i) identifying the set in the unrelated sample set that have the most related samples in the unrelated sample set, thereby designated as X, (ii) identifying the set of samples in X that have the least ancestry-diverged pairings compared to samples in the unrelated sample set, thereby designated as Y; and (iii) if Y has zero samples, then terminating the process, or, if Y has at least one sample, then randomly selecting one sample from Y to remove from U, and repeat beginning at step (3) (i).

61. The method of claim 57 or claim 58, wherein the PCA method is a modified PC-Air.

62. The method of claim 61, wherein the modified PC-AiR comprises steps of: (1) estimating kinship coefficients between all pairs of samples, optionally training DNA profiles, of a training database, wherein pairings with a kinship coefficient >0.01 are identified as related and pairings with a kinship coefficient <−0.025 are identified as ancestry-diverged; (2) removing all DNA profiles that have ≥5% missing data; and (3) ranking all DNA profiles by identifying each DNA profile with a ranking value. In some embodiments, the ranking value is determined based on the number of related DNA profiles in the full database that is ranked from least to most, and ties are broken by the number of ancestry-diverged DNA profiles in the full database as ranked from most to least. In some embodiments, step (3) involves going iteratively through the ranked DNA profiles, and for each DNA profile: (i) if the DNA profile is not yet in the related sample set, adding it to the unrelated sample set and adding all related DNA profiles to the related sample set, and (ii) if the DNA profile is already in the related sample set, then skipping to the next DNA profile, and repeating beginning at step (3) (i).

63. The method of any one of claims 1-62, wherein the calculating the degree of relationship comprises calculating a kinship coefficient using PC-Relate.

64. The method of claim 63, wherein the degree of relationship is calculated by providing the DNA profile of the person of interest as input to PC-Relate.

65. The method of any one of claims 57-64, wherein the degree of relationship is calculated by providing the kinship model and the DNA profile of the person of interest as input to PC-Relate.

66. The method of any one of claims 63-65, wherein the one or more reference DNA profiles are further provided as input to PC-Relate.

67. The method of any one of claims 1-66, wherein the calculating the degree of relationship comprises calculating a kinship coefficient using a whole genome kinship alogirthm as follows:
$$\text{?} = \frac{{\sum }_{s ∈ S} ( {g}_{i s} - 2 {\hat{u}}_{i s} ) ( {g}_{j s} - 2 {\hat{u}}_{j s} )}{4 {{\sum }_{s ∈ S} [ {\hat{u}}_{i s} ( 1 - {\hat{u}}_{i s} ) \cdot {\hat{u}}_{j s} ( 1 - {\hat{u}}_{j s} ) ]}^{1 / 2}}$$
wherein the person of interest and a reference DNA profile of the one or more reference DNA profiles are i and j, [symbol] is the kinship coefficient, û is the estimated allele frequencies, s is a SNP in S SNPs that were typed in both individuals, gᵢₛ and gⱼₛ are the number of reference alleles in i and j at SNP s, respectively, and ûᵢₛ and ûⱼₛ are the expected allele frequencies calculated by PC-AiR for i and j at SNP s, respectively.

68. The method of any one of claims 1-67, wherein the calculating the degree of relationship comprises calculating a likelihood ratio.

69. The method of claim 68, wherein the calculating the likelihood ratio comprises comparing the plurality of SNPs between the DNA profile and the one or more reference DNA profiles.

70. The method of claim 68, wherein the calculating the likelihood ratio comprises comparing a set of SNPs comprising kinship SNPs from within the plurality of SNPs between the DNA profile and the one or more reference DNA profiles.

71. The method of any one of claims 68-70, wherein calculating the likelihood ratio comprises dividing the probability of the DNA profile and a reference DNA profile from among the one or more reference DNA profiles being related by the probability of the DNA profile and the reference DNA profile being unrelated based on the genotypes of the plurality of SNPs.

72. The method of any one of claims 68-71, wherein the likelihood ratio (LR) is calculated as follows:
$$L R = \frac{P ( D \vert {H}_{r} )}{P ( D \vert {H}_{u} )}$$
wherein D represents the genotypes, Hᵣ represents the hypothesis that the individuals are related, and Hᵤ represents the hypothesis that the individuals are unrelated.

73. The method of any one of claims 68-71, wherein the LR is calculated as as follows:
$$L R = \frac{0.001}{{p}^{2} {q}^{2}}$$
wherein 0.001 represents a genotyping error rate, p is the allele frequency of the allele 1, and q is the allele frequency of allele 2.

74. The method of any one of claims 1-73, wherein the person of interest is biologically male and the method further comprises calculating a likelihood ratio for sharing a Y chromosome between the DNA profile and the one or more reference DNA profiles.

75. The method of claim 74, wherein the calculating a likelihood ratio for sharing a Y chromosome comprises comparing a set of SNPs that comprises one or more Y-SNPs between the DNA profile and the one or more reference DNA profiles.

76. The method of claim 75, wherein the one or more Y-SNPs are comprised within the plurality of SNPs.

77. The method of claim 75, wherein the one or more Y-SNPs comprises at least 20, 25, 30, 35, 40, 45, 50, 55, 60, 65, 70, 75, 80, 81, 82, 83, 84, or 85 Y-SNPs.

78. The method of any one of claims 75-77, wherein the one or more Y-SNPs comprises 85 Y-SNPs.

79. The method of any one of claims 75-78, wherein calculating the likelihood ratio for sharing a Y chromosome comprises dividing the probability of the DNA profile and a reference DNA profile from among the one or more reference DNA profiles sharing a Y chromosome by the probability of the DNA profile and the reference DNA profile not sharing a Y chromosome based on the genotypes of the one or more Y-SNPs.

80. The method of any one of claims 1-79, wherein at least 90%, 91%, 92%, 93%, 94%, 95%, 96%, 97%, 98%, or 99% of the DNA profiles in the reference set of DNA profiles is from a relative of a missing person or a victim of a disaster or a conflict.

81. The method of any one of claims 1-80, wherein each of the DNA profiles in the reference set of DNA profiles is from a relative of a missing person or a victim of a disaster or a conflict.

82. The method of any one of claims 1-81, wherein the reference set of DNA profiles comprises up to 5, 10, 15, 20, 25, 30, 35, 40, 45, 50, 55, 60, 65, 70, 75, 80, 85, 90, 95, 100, 125, 150, 175, 200, 300, 400, 500, 600, 700, 800, 900, or 1000 reference DNA profiles.

83. The method of any one of claims 1-82, wherein the reference set of DNA profiles comprises up to 100 reference DNA profiles.

84. The method of any one of claims 1-83, wherein at least 5%, 10%, 15%, 20%, 25%, 30%, 35%, 40%, 45%, 50%, 55%, 60%, 65%, 70%, 75%, 80%, 85%, 90%, or 95% of the reference DNA profiles in the reference set of DNA profiles is from a relative of the person of interest.

85. The method of any one of claims 1-84, wherein at least 50% of the reference DNA profiles in the reference set of DNA profiles is from a relative of the person of interest.

86. The method of claim 84 or claim 85, wherein each relative of the person of interest in the reference set of DNA profiles is individually a first degree, second degree, third degree, fourth degree, or fifth degree relative of the person of interest.

87. The method of any one of claims 1-86, wherein the identity of each relative of the person of interest in the reference set of DNA profiles is known.

88. The method of any one of claims 1-87, wherein the identity of each relative of the person of interest in the reference set of DNA profiles is known.

89. The method of any one of claims 1-88, wherein the identity of each of the one or more reference DNA profiles in the reference set of DNA profiles is known.

90. The method of any one of claims 1-89, wherein the reference set of DNA profiles is in a database.

91. The method of claim 90, wherein the database is not publicly accessible.

92. The method of claim 90 or claim 91, wherein the database is not accessible by a third party geneaological service.

93. A nucleic acid library constructed using the method of any one of claims 6-92.

94. A plurality of primers that specifically hybridize to a plurality of target sequences comprising at least between at or about 2,000 to 50,000 single nucleotide polymorphisms (SNPs) in a nucleic acid sample from a person of interest, wherein amplifying the nucleic acid sample using the plurality of primers in one or more multiplex PCR reactions results in amplification products.

95. A plurality of primers that specifically hybridize to a plurality of target sequences comprising at least between at or about 2,000 to 50,000 single nucleotide polymorphisms (SNPs) in a nucleic acid sample from a person of interest and a nucleic acid sample from one or more reference samples,
wherein the one or more reference samples comprises a sample from a relative of the person of interest, and
wherein amplifying the nucleic acid sample from the person of interest and the nucleic acid sample from one or more reference samples using the plurality of primers in one or more multiplex PCR reactions results in amplification products.

96. The plurality of primers of claim 94 or claim 95, wherein the nucleic acid sample from the person of interest comprises genomic DNA.

97. The plurality of primers of any one of claims 94-96, wherein the nucleic acid sample from the person of interest comprises one or more enzyme inhibitors.

98. The plurality of primers of claim 97, wherein the one or more enzyme inhibitors comprise one or more inhibitors selected from the group consisting of hematin, heme, humic acid, indigo, tannic acid, collagen, calcium, and hydroxyapatite.

99. The plurality of primers of any one of claims 94-98, wherein the nucleic acid sample from the person of interest comprises low-quality nucleic acid molecules and/or low quantity nucleic acid molecules.

100. The plurality of primers of claim 99, wherein the low quality nucleic acid molecules are degraded genomic DNA and/or fragmented genomic DNA.

101. The plurality of primers of claim 99 or claim 100, wherein the low quality nucleic acid molecules have a degradation index (DI) of at or at least 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200.

102. The plurality of primers of any one of claims 99-101, wherein the low quality nucleic acid molecules have a DI of at least 1 and up to or less than 158.3.

103. The plurality of primers of any one of claims 94-102, wherein the nucleic acid sample from the person of interest and/or the nucleic acid sample from one or more reference samples comprises high quality nucleic acid molecules.

104. The plurality of primers of claim 103, wherein the high quality nucleic acid molecules have a DI of less than 1.

105. The plurality of primers of any one of claims 94-104, wherein the person of interest is a missing person.

106. The plurality of primers of any one of claims 94-104, wherein the person of interest is a victim of a disaster or conflict.

107. The plurality of primers of any one of claims 94-106, wherein the nucleic acid sample from the person of interest is derived from a buccal swab, paper, fabric, or other substrate or object that is impregnated with saliva, blood, or other bodily fluid, or contains hair or skin cells.

108. The plurality of primers of any one of claims 94-107, wherein the nucleic acid sample from the person of interest comprises between or between about 3 pg and 100 ng of genomic DNA.

109. The plurality of primers of any one of claims 94-108, wherein the nucleic acid sample from the person of interest comprises between or between about 100 pg and 5 ng of genomic DNA, between or between about 50 pg and 5 ng of genomic DNA, or between or between about 3 pg and 5 ng of genomic DNA.

110. The plurality of primers of claim 108 or claim 109, wherein the nucleic acid sample from the person of interest comprises at or about 1 ng of genomic DNA.

111. The plurality of primers of any one of claims 94-110, wherein the plurality of SNPs comprises kinship SNPs (kiSNPs).

112. The method of any one of claims 94-111, wherein the plurality of SNPs comprises Y-chromosome SNPs (Y-SNPs).

113. The method of any one of claims 94-112, wherein the plurality of SNPs comprises kiSNPs and Y-SNPs.

114. The plurality of primers of any one of claims 94-113, wherein the plurality of SNPs comprises kiSNPs, biogeographical ancestry SNPs (aiSNPs), identity SNPs (iiSNPs), phenotype SNPs (piSNPs), X-chromosome SNPs (X-SNPs), and Y-chromosome SNPs (Y-SNPs).

115. The plurality of primers of any one of claims 94-111, wherein the plurality of SNPs comprises SNPs selected from one or more of the groups consisting of kiSNPs, aiSNPs, iiSNPs, piSNPs, X-SNPs, and Y-SNPs.

116. The plurality of primers of any one of claims 94-115, wherein at least or at least about 80%, 81%, 82%, 83%, 84%, 85%, 86%, 87%, 88%, 89%, 90%, 91%, 92%, 93%, 94%, 95%, 96%, 97%, 98%, or 99% of the plurality of SNPs are kinship SNPs.

117. The plurality of primers of any one of claims 94-116, wherein at least 90%, 91%, 92%, 93%, 94%, 95%, 96%, 97%, 98%, or 99% of the DNA profiles in the reference set of DNA profiles is from a relative of a missing person or a victim of a disaster or a conflict.

118. The plurality of primers of any one of claims 94-116, wherein each of the one or more references samples is from a relative of a missing person or a victim of a disaster or a conflict.

119. The plurality of primers of any one of claims 94-118, wherein the one or more reference samples comprises up to 5, 10, 15, 20, 25, 30, 35, 40, 45, 50, 55, 60, 65, 70, 75, 80, 85, 90, 95, 100, 125, 150, 175, 200, 300, 400, 500, 600, 700, 800, 900, or 1000 reference samples.

120. The plurality of primers of any one of claims 94-119, wherein at least 5%, 10%, 15%, 20%, 25%, 30%, 35%, 40%, 45%, 50%, 55%, 60%, 65%, 70%, 75%, 80%, 85%, 90%, or 95% of the one or more reference samples is from a relative of the person of interest.

121. The plurality of primers of any one of claims 94-120, wherein at least 5%, 10%, 15%, 20%, 25%, 30%, 35%, 40%, 45%, 50%, 55%, 60%, 65%, 70%, 75%, 80%, 85%, 90%, or 95% of the reference DNA profiles in the reference set of DNA profiles is from a relative of the person of interest, and wherein each of the at least 5%, 10%, 15%, 20%, 25%, 30%, 35%, 40%, 45%, 50%, 55%, 60%, 65%, 70%, 75%, 80%, 85%, 90%, or 95% of the reference DNA profiles in the reference set of DNA profiles is a first degree, second degree, third degree, fourth degree, or fifth degree relative.

122. The plurality of primers of any one of claims 94-121, wherein at least 50% of the one or more reference samples is from a relative of the person of interest.

123. The plurality of primers of any one of claims 120-122, wherein each relative of the person of interest in the one or more reference samples is individually a first degree, second degree, third degree, fourth degree, or fifth degree relative of the person of interest.

124. The plurality of primers of claim 123, wherein each relative of the person of interest in the one or more reference samples is individually a first degree, second degree, or third degree relative of the person of interest.

125. The plurality of primers of any one of claims 94-124, wherein the identity of each relative of the person of interest in the one or more reference samples is known.

126. The plurality of primers of any one of claims 94-125, wherein the identity of each of the one or more reference samples is known.

127. A method for constructing a DNA profile, comprising:
providing a nucleic acid sample from a person of interest,
amplifying the nucleic acid sample with a plurality of primers that specifically hybridize to a plurality of target sequences collectively comprising a plurality of at least between at or about 2,000 to 50,000 single nucleotide polymorphisms (SNPs), thereby generating amplification products, wherein the amplification is carried out in one or more multiplex PCR reactions,
sequencing the amplification products,
determining the genotypes of the plurality of SNPs, thereby generating a DNA profile.

128. A method for constructing a DNA profile, comprising:
providing a nucleic acid sample from a person of interest,
providing a nucleic acid sample from a relative of the person of interest,
amplifying the nucleic acid sample from the person of interest and the nucleic acid sample from the relative with a plurality of primers that specifically hybridize to a plurality of target sequences collectively comprising a plurality of at least between at or about 2,000 to 50,000 single nucleotide polymorphisms (SNPs), thereby generating amplification products, wherein the amplification is carried out in one or more multiplex PCR reactions,
sequencing the amplification products,
determining the genotypes of the plurality of SNPs, thereby generating a DNA profile for the person of interest and the relative of the person of interest.

129. The method of claim 127 or claim 128, wherein the sequencing does not comprise whole genome sequencing (WGS).

130. The method of claim 127 or claim 129, wherein the nucleic acid sample comprises genomic DNA.

131. The method of claim 128 or claim 129, wherein the nucleic acid sample of the person of interest and/or the nucleic acid sample of the relative of the person of interest comprises genomic DNA.

132. The method of any one of claims 127-131, wherein the nucleic acid sample, the nucleic acid sample of the person of interest, and/or the nucleic acid sample of the relative comprises one or more enzyme inhibitors.

133. The method of claim 132, wherein the one or more enzyme inhibitors comprise one or more inhibitors selected from the group consisting of hematin, heme, humic acid, indigo, tannic acid, collagen, calcium, and hydroxyapatite.

134. The method of any one of claims 127-133, wherein the nucleic acid sample, the nucleic acid sample of the person of interest, and/or the nucleic acid sample of the relative comprises low-quality nucleic acid molecules and/or low quantity nucleic acid molecules.

135. The method of claim 134, wherein the low quality nucleic acid molecules are degraded genomic DNA and/or fragmented genomic DNA.

136. The method of claim 134 or claim 135, wherein the low quality nucleic acid molecules have a degradation index (DI) of at or at least 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200.

137. The method of any one of claims 134-136, wherein the low quality nucleic acid molecules have a DI of at least 1 and up to or less than 158.3.

138. The method of any one of claims 127-133, wherein the nucleic acid sample, the nucleic acid sample of the person of interest, and/or the nucleic acid sample of the relative comprises high quality nucleic acid molecules.

139. The method of claim 138, wherein the high quality nucleic acid molecules have a DI of less than 1.

140. The method of any one of claims 127-139, wherein the person of interest is a missing person.

141. The method of any one of claims 127-139, wherein the person of interest is a victim of a disaster or conflict.

142. The method of any one of claims 128, 129, and 131-141, wherein the relative of the person of interest is a first-, second-, third-, fourth-, or fifth-degree relative.

143. The method of any one of claims 128, 129, and 131-141, wherein the relative of the person of interest is a first-, second-, or third-degree relative.

144. The method of any one of claims 127-143, wherein the nucleic acid sample, the nucleic acid sample of the person of interest, and/or the nucleic acid sample of the relative is derived from a buccal swab, paper, fabric, or other substrate or object that is impregnated with saliva, blood, or other bodily fluid, or contains hair or skin cells.

145. The method of any one of claims 127-144, wherein the nucleic acid sample, the nucleic acid sample of the person of interest, and/or the nucleic acid sample of the relative comprises between or between about 3 pg and 100 ng of genomic DNA.

146. The method of any one of claims 127-145, wherein the nucleic acid sample, the nucleic acid sample of the person of interest, and/or the nucleic acid sample of the relative comprises between or between about 100 pg and 5 ng of genomic DNA, between or between about 50 pg and 5 ng of genomic DNA, or between or between about 3 pg and 5 ng of genomic DNA.

147. The method of claim 145 or claim 146, wherein the nucleic acid sample, the nucleic acid sample of the person of interest, and/or the nucleic acid sample of the relative comprises at or about 1 ng of genomic DNA.

148. The method of any one of claims 127-147 wherein the plurality of SNPs comprises kinship SNPs.

149. The method of any one of claims 127-148, wherein the plurality of SNPs comprises Y-chromosome SNPs (Y-SNPs).

150. The method of any one of claims 127-149, wherein the plurality of SNPs comprises kiSNPs and Y-SNPs.

151. The method of any one of claims 127-150, wherein the plurality of SNPs comprises kiSNPs, biogeographical ancestry SNPs (aiSNPs), identity SNPs (iiSNPs), phenotype SNPs (piSNPs), X-chromosome SNPs (X-SNPs), and Y-chromosome SNPs (Y-SNPs).

152. The method of any one of claims 127-151, wherein the plurality of SNPs comprises SNPs selected from one or more of the groups consisting of kiSNPs, aiSNPs, iiSNPs, piSNPs, X-SNPs, and Y-SNPs.

153. The method of any one of claims 127-152, wherein at least or at least about 80%, 81%, 82%, 83%, 84%, 85%, 86%, 87%, 88%, 89%, 90%, 91%, 92%, 93%, 94%, 95%, 96%, 97%, 98%, or 99% of the plurality of SNPs are kinship SNPs.

154. The method of any one of claims 1-92 and 127-153, wherein the sequencing comprises a sequencing plexity of up to 40-plex.

155. The method of any one of claims 1-92 and 127-153, wherein the sequencing comprises a sequencing plexity of up to 32-plex.

156. The method of any one of claims 1-92 and 127-153, wherein the sequencing comprises a sequencing plexity of 12-plex to 32-plex.

157. The method of any one of claims 1-92 and 127-153, wherein the sequencing comprises a sequencing plexity of 24-plex to 32-plex.

158. The method of any one of claims 1-92 and 127-153, wherein:
the sequencing comprises a sequencing plexity of at or about 4-plex, 5-plex, 6-plex, 7-plex, 8-plex, 9-plex, 10-plex, 11-plex, 12-plex, 13-plex, 14-plex, 15-plex, 16-plex, 17-plex 18-plex, 19-plex, 20-plex, 21-plex, 22-plex, 23-plex, 24-plex, 25-plex, 26-plex, 27-plex, 28-plex, 29-plex, 30-plex, 31-plex, 32-plex, 33-plex, 34-plex, 35-plex, 36-plex, 37-plex, 38-plex, 39-plex, 40-plex, 41-plex, 42-plex, 43-plex, 44-plex, or 45-plex; or
the sequencing comprises a sequencing plexity of at or about 10-plex, 11-plex, 12-plex, 13-plex, 14-plex, 15-plex, 16-plex, 17-plex 18-plex, 19-plex, 20-plex, 21-plex, 22-plex, 23-plex, 24-plex, 25-plex, 26-plex, 27-plex, 28-plex, 29-plex, 30-plex, 31-plex, 32-plex, 33-plex, 34-plex, or 35-plex.

159. The method of any one of claims 1-92 and 127-158, wherein the sequencing comprises a sequencing plexity of at or about 8-to 16-plex for postmortem samples, and/or the sequencing comprises a sequencing plexity of at or about 24-to 40-plex for antemortem samples.

160. The method of any one of claims 1-92 and 127-158, wherein the sequencing comprises a sequencing plexity of at or about 12-plex for postmortem samples, and/or the sequencing comprises a sequencing plexity of at or about 32-plex for antemortem samples.

161. The method of any one of claims 1-92 and 127-153, wherein the sequencing comprises a sequencing plexity of at or about 30-plex, 31-plex, or 32-plex.

162. A method of identifying genetic relatives of a DNA profile, comprising:
calculating the degree of relationship of the DNA profile of any one of claims 127-161 to one or more reference DNA profiles, wherein the one or more reference DNA profiles are comprised within a reference set of DNA profiles comprising one or more reference DNA profiles from a relative of the person of interest; and
generating a family tree comprising the DNA profile in relation to the one or more reference DNA profiles.

163. The method of claim 162, wherein the one or more reference DNA profiles are part of a database.

164. The method of claim 162 or claim 163, wherein the reference set of DNA profiles comprises up to 5, 10, 15, 20, 25, 30, 35, 40, 45, 50, 55, 60, 65, 70, 75, 80, 85, 90, 95, 100, 125, 150, 175, 200, 300, 400, 500, 600, 700, 800, 900, or 1000 reference DNA profiles.

165. The method of any one of claims 162-164, wherein at least 5%, 10%, 15%, 20%, 25%, 30%, 35%, 40%, 45%, 50%, 55%, 60%, 65%, 70%, 75%, 80%, 85%, 90%, or 95% of the reference DNA profiles in the reference set of DNA profiles is from a relative of the person of interest.

166. The method of any one of claims 162-165, wherein at least 50% of the reference DNA profiles in the reference set of DNA profiles is from a relative of the person of interest.

167. The method of claim 165 or claim 166, wherein each relative of the person of interest is individually a first degree, second degree, third degree, fourth degree, or fifth degree relative of the person of interest.

168. The method of any one of claims 162-167, wherein the identity of each relative of the person of interest in the reference set of DNA profiles is known.

169. The method of any one of claims 162-168, wherein the identity of each of the one or more reference DNA profiles in the reference set of DNA profiles is known.

170. The method of any one of claims 162-169, wherein the reference set of DNA profiles is in a database.

171. The method of claim 170, wherein the database is not publicly accessible.

172. The method of claim 170 or claim 171, wherein the database is not accessible by a third party geneaological service.

173. A method of identifying the identity of a DNA profile, comprising:
calculating the degree of relationship of a DNA profile comprising genotypes of at least between at or about 2,000 to 50,000 SNPs to one or more reference DNA profiles, wherein the DNA profile is from a person of interest, and wherein the one or more reference DNA profiles are comprised within a reference set of DNA profiles comprising one or more reference DNA profiles from a relative of the person of interest; and
generating a family tree comprising the DNA profile in relation to the one or more reference DNA profiles.

174. The method of claim 173, wherein the DNA profile is generated by the method of any one of claims 127-161.

175. The method of claim 173 or claim 174, wherein the degree of relationship is calculated using a kinship model.

176. The method of any one of claims 173-175, wherein the degree of relationship is calculated using a kinship model that is trained using a PCA method.

177. The method of claim 176, wherein the PCA method for training the kinship model is PCA or involves PCA.

178. The method of claim 176 or claim 177, wherein the PCA method is PC-AiR.

179. The method of claim 178, wherein the PC-AiR comprises the steps of: (1) estimating kinship coefficients between all pairs of samples, optionally training DNA profiles, of a training database, wherein pairings with a kinship coefficient >0.025 are identified as related and pairings with a kinship coefficient <−0.025 are identified as ancestry-diverged; (2) initializing an unrelated sample set that includes all samples; and (3) iteratively: (i) identifying the set in the unrelated sample set that have the most related samples in the unrelated sample set, thereby designated as X, (ii) identifying the set of samples in X that have the least ancestry-diverged pairings compared to samples in the unrelated sample set, thereby designated as Y; and (iii) if Y has zero samples, then terminating the process, or, if Y has at least one sample, then randomly selecting one sample from Y to remove from U, and repeat beginning at step (3) (i).

180. The method of claim 176 or claim 177, wherein the PCA method is a modified PC-Air.

181. The method of claim 180, wherein the modified PC-AiR comprises steps of: (1) estimating kinship coefficients between all pairs of samples, optionally training DNA profiles, of a training database, wherein pairings with a kinship coefficient >0.01 are identified as related and pairings with a kinship coefficient <−0.025 are identified as ancestry-diverged; (2) removing all DNA profiles that have ≥5% missing data; and (3) ranking all DNA profiles by identifying each DNA profile with a ranking value. In some embodiments, the ranking value is determined based on the number of related DNA profiles in the full database that is ranked from least to most, and ties are broken by the number of ancestry-diverged DNA profiles in the full database as ranked from most to least. In some embodiments, step (3) involves going iteratively through the ranked DNA profiles, and for each DNA profile: (i) if the DNA profile is not yet in the related sample set, adding it to the unrelated sample set and adding all related DNA profiles to the related sample set, and (ii) if the DNA profile is already in the related sample set, then skipping to the next DNA profile, and repeating beginning at step (3) (i).

182. The method of any one of claims 173-181, wherein the calculating the degree of relationship comprises calculating a kinship coefficient using PC-Relate.

183. The method of claim 182, wherein the degree of relationship is calculated by providing the DNA profile of the person of interest as input to PC-Relate.

184. The method of claim 182 or claim 183, wherein the degree of relationship is calculated by providing the kinship model and the DNA profile of the person of interest as input to PC-Relate.

185. The method of any one of claims 182-184, wherein the one or more reference DNA profiles are further provided as input to PC-Relate.

186. The method of any one of claims 173-185, wherein the calculating the degree of relationship comprises calculating a kinship coefficient using a whole genome kinship alogirthm as follows:
$$\text{?} = \frac{{\sum }_{s ∈ S} ( {g}_{i s} - 2 {\hat{u}}_{i s} ) ( {g}_{j s} - 2 {\hat{u}}_{j s} )}{4 {{\sum }_{s ∈ S} [ {\hat{u}}_{i s} ( 1 - {\hat{u}}_{i s} ) \cdot {\hat{u}}_{j s} ( 1 - {\hat{u}}_{j s} ) ]}^{1 / 2}}$$
wherein the person of interest and a reference DNA profile of the one or more reference DNA profiles are i and j, [symbol] is the kinship coefficient, û is the estimated allele frequencies, s is a SNP in S SNPs that were typed in both individuals, gᵢₛ and gⱼₛ are the number of reference alleles in i and j at SNP s, respectively, and ûᵢₛ and ûⱼₛ are the expected allele frequencies calculated by PC-AiR for i and j at SNP s, respectively.

187. The method of any one of claims 173-186, wherein the calculating the degree of relationship comprises calculating a likelihood ratio.

188. The method of claim 187, wherein the calculating the likelihood ratio comprises comparing the plurality of SNPs between the DNA profile and the one or more reference DNA profiles.

189. The method of claim 187, wherein the calculating the likelihood ratio comprises comparing a set of SNPs comprising kinship SNPs from within the plurality of SNPs between the DNA profile and the one or more reference DNA profiles.

190. The method of any one of claims 187-189, wherein calculating the likelihood ratio comprises dividing the probability of the DNA profile and a reference DNA profile from among the one or more reference DNA profiles being related by the probability of the DNA profile and the reference DNA profile being unrelated based on the genotypes of the plurality of SNPs.

191. The method of any one of claims 187-190, wherein the likelihood ratio (LR) is calculated as follows:
$$L R = \frac{P ( D \vert {H}_{r} )}{P ( D \vert {H}_{u} )}$$
wherein D represents the genotypes, Hᵣ represents the hypothesis that the individuals are related, and Hᵤ represents the hypothesis that the individuals are unrelated.

192. The method of any one of claims 187-190, wherein the LR is calculated as as follows:
$$L R = \frac{0.001}{{p}^{2} {q}^{2}}$$
wherein 0.001 represents a genotyping error rate, p is the allele frequency of the allele 1, and q is the allele frequency of allele 2.

193. The method of any one of claims 173-192, wherein the person of interest is biologically male and the method further comprises calculating a likelihood ratio for sharing a Y chromosome between the DNA profile and the one or more reference DNA profiles.

194. The method of claim 193, wherein the calculating a likelihood ratio for sharing a Y chromosome comprises comparing a set of SNPs that comprises one or more Y-SNPs between the DNA profile and the one or more reference DNA profiles.

195. The method of claim 194, wherein the one or more Y-SNPs are comprised within the plurality of SNPs.

196. The method of claim 194 or claim 195, wherein the one or more Y-SNPs comprises at least 20, 25, 30, 35, 40, 45, 50, 55, 60, 65, 70, 75, 80, 81, 82, 83, 84, or 85 Y-SNPs.

197. The method of any one of claims 194-196, wherein the one or more Y-SNPs comprises 85 Y-SNPs.

198. The method of any one of claims 193-197, wherein calculating the likelihood ratio for sharing a Y chromosome comprises dividing the probability of the DNA profile and a reference DNA profile from among the one or more reference DNA profiles sharing a Y chromosome by the probability of the DNA profile and the reference DNA profile not sharing a Y chromosome based on the genotypes of the one or more Y-SNPs.

199. The method of any one of claims 173-198, wherein at least 90%, 91%, 92%, 93%, 94%, 95%, 96%, 97%, 98%, or 99% of the DNA profiles in the reference set of DNA profiles is from a relative of a missing person or a victim of a disaster or a conflict.

200. The method of any one of claims 173-199, wherein each of the one or more references samples is from a relative of a missing person or a victim of a disaster or a conflict.

201. The method of any one of claims 173-200, wherein the one or more reference samples comprises up to 5, 10, 15, 20, 25, 30, 35, 40, 45, 50, 55, 60, 65, 70, 75, 80, 85, 90, 95, 100, 125, 150, 175, 200, 300, 400, 500, 600, 700, 800, 900, or 1000 reference samples.

202. The method of any one of claims 173-201, wherein at least 5%, 10%, 15%, 20%, 25%, 30%, 35%, 40%, 45%, 50%, 55%, 60%, 65%, 70%, 75%, 80%, 85%, 90%, or 95% of the one or more reference samples is from a relative of the person of interest.

203. The method of any one of claims 173-202, wherein at least 5%, 10%, 15%, 20%, 25%, 30%, 35%, 40%, 45%, 50%, 55%, 60%, 65%, 70%, 75%, 80%, 85%, 90%, or 95% of the reference DNA profiles in the reference set of DNA profiles is from a relative of the person of interest, and wherein each of the at least 5%, 10%, 15%, 20%, 25%, 30%, 35%, 40%, 45%, 50%, 55%, 60%, 65%, 70%, 75%, 80%, 85%, 90%, or 95% of the reference DNA profiles in the reference set of DNA profiles is a first degree, second degree, third degree, fourth degree, or fifth degree relative.

204. The method of any one of claims 173-203, wherein at least 50% of the one or more reference samples is from a relative of the person of interest.

205. The method of any one of claims 199-204, wherein each relative of the person of interest in the one or more reference samples is individually a first degree, second degree, third degree, fourth degree, or fifth degree relative of the person of interest.

206. The method of claim 205, wherein each relative of the person of interest in the one or more reference samples is individually a first degree, second degree, or third degree relative of the person of interest.

207. The method of any one of claims 173-206, wherein the identity of each relative of the person of interest in the one or more reference samples is known.

208. The method of any one of claims 173-207, wherein the identity of each of the one or more reference samples is known.

209. A kit comprising at least one container means, wherein the at least one container means comprises a plurality of primers of any one of claims 94-126.

210. The method of any one of claims 1-92 and 127-208, wherein the plurality of SNPs comprises between or between about 2,000 to 11,000 SNPs, 3,000 to 11,000 SNPs, 4,000 to 11,000 SNPs, 5,000 to 11,000 SNPs, 5,500 to 11,000 SNPs, 6,000 to 11,000 SNPs, 7,000 to 15,000 SNPs, 7,000 to 14,000 SNPs, 7,000 to 13,000 SNPs, 7,000 to 12,000 SNPs, 7,000 to 11,000 SNPs, 8,000 to 15,000 SNPs, 8,000 to 14,000 SNPs, 8,000 to 13,000 SNPs, 8,000 to 12,000 SNPs, 8,000 to 11,000 SNPs, 9,000 to 15,000 SNPs, 9,000 to 14,000 SNPs, 9,000 to 13,000 SNPs, 9,000 to 12,000 SNPs, or 9,000 to 11,000 SNPs.

211. The method of any one of claims 1-92 and 127-208, wherein the plurality of SNPs comprises 10,230 SNPs.

212. The plurality of primers of any one of claims 94-126, wherein the plurality of SNPs comprises between or between about 2,000 to 11,000 SNPs, 3,000 to 11,000 SNPs, 4,000 to 11,000 SNPs, 5,000 to 11,000 SNPs, 5,500 to 11,000 SNPs, 6,000 to 11,000 SNPs, 7,000 to 15,000 SNPs, 7,000 to 14,000 SNPs, 7,000 to 13,000 SNPs, 7,000 to 12,000 SNPs, 7,000 to 11,000 SNPs, 8,000 to 15,000 SNPs, 8,000 to 14,000 SNPs, 8,000 to 13,000 SNPs, 8,000 to 12,000 SNPs, 8,000 to 11,000 SNPs, 9,000 to 15,000 SNPs, 9,000 to 14,000 SNPs, 9,000 to 13,000 SNPs, 9,000 to 12,000 SNPs, or 9,000 to 11,000 SNPs.

213. The plurality of primers of any one of claims 94-126, wherein the plurality of SNPs comprises 10,230 SNPs.

214. The method of any one of claims 1-5, 11-93, 127-161, and 210-213, further comprising generating a family tree comprising the DNA profile in relation to one or more DNA profiles comprised in the reference set of DNA profiles.

215. The method of claim 214, wherein the family tree comprises the DNA profile in relation to one or more DNA profiles from a relative of the person of interest.
